SEQUENCE LISTING (10 sequences)

SEQ ID NO: 1 - DNA, 20 bp, synthetic construct
  source 1..20: mol_type = other DNA
cacatgccacgttggtgttt

SEQ ID NO: 2 - DNA, 20 bp, synthetic construct
  source 1..20: mol_type = other DNA
acgatccgggcaatttccat

SEQ ID NO: 3 - DNA, 21 bp, synthetic construct
  source 1..21: mol_type = other DNA
cagtacagccgggaagacaat

SEQ ID NO: 4 - DNA, 20 bp, synthetic construct
  source 1..20: mol_type = other DNA
ttggcaacccaagtaaccct

SEQ ID NO: 5 - DNA, 20 bp, synthetic construct
  source 1..20: mol_type = other DNA
tatctatgctcgccttgcgg

SEQ ID NO: 6 - DNA, 20 bp, synthetic construct
  source 1..20: mol_type = other DNA
gacacagagacggctggaac

SEQ ID NO: 7 - DNA, 20 bp, synthetic construct
  source 1..20: mol_type = other DNA
gcagatggatggacgaatgg

SEQ ID NO: 8 - DNA, 21 bp, synthetic construct
  source 1..21: mol_type = other DNA
cgaagcacactttgagtttgg

SEQ ID NO: 9 - DNA, 20 bp, synthetic construct
  source 1..20: mol_type = other DNA
ctcggtgaccctggtctttc

SEQ ID NO: 10 - DNA, 20 bp, synthetic construct
  source 1..20: mol_type = other DNA
ggatttcaatgtgaggcggg

(19) United States
(12) Patent Application Publication
(10) Pub. No.: US 2023/0414615 A1
(43) Pub. Date: Dec. 28, 2023

(54) PYRIMIDINONE COMPOUNDS FOR TREATING ACUTE INFLAMMATION

(71) Applicant: TES Pharma S.r.l., Corciano (IT)

(72) Inventors: Roberto PELLICCIARI, Perugia (IT); Nicola GIACCHÈ, Perugia (IT); Francesca DE FRANCO, Magione (IT)

(21) Appl. No.: 18/201,674

(22) Filed: May 24, 2023

Related U.S. Application Data
(60) Provisional application No. 63/346,193, filed on May 26, 2022.

(51) Int. Cl. A61K 31/513; A61K 31/505; A61P 29/00

(52) U.S. Cl. CPC A61K 31/513; A61K 31/505; A61P 29/00

(57) ABSTRACT

The present disclosure relates to a method of treating an acute inflammatory condition in a subject comprising administering to the subject a therapeutically effective amount of a pyrimidinone compound.

### CROSS REFERENCE TO RELATED APPLICATIONS

This application claims the benefit of U.S. Provisional Application No. 63/346,193, filed May 26, 2022, the contents of which is incorporated herein by reference in its entirety.

### FIELD OF THE DISCLOSURE

The present disclosure relates to a method of treating an acute inflammatory condition in a subject comprising administering to the subject a therapeutically effective amount of a pyrimidinone compound.

### BACKGROUND OF THE DISCLOSURE

Inflammation is a complex of sequential biological responses of body tissues to harmful stimuli, such as pathogens, damaged cells, or irritants. When tissue injury occurs, whether it is caused by bacteria, trauma, chemicals, heat, or any other phenomenon, histamine, along with other humoral substances, is liberated by the damaged tissue into the surrounding fluids and initiates the vascular phase of acute inflammation. This is a protective adaptation by the organism to remove the injurious stimuli as well as initiating the healing process.

There are two forms of inflammation, commonly referred to as acute inflammation and chronic inflammation. Acute inflammation is the initial response of the body to harmful stimuli and is achieved by the increased movement of plasma and leukocytes from the blood into the injured tissues. Acute inflammation can be divided into several phases. The earliest, event of an inflammatory response is temporary vasoconstriction, i. e., narrowing of blood vessels caused by contraction of smooth muscle in the vessel walls which can be seen as blanching (whitening) of the skin. This is followed by several phases that occur minutes, hours, and days later. The first is the acute vascular response which follows within seconds of the tissue injury and lasts for several minutes. This results from vasodilation and increased capillary permeability due to alterations in the vascular endothelium which leads to increased blood flow (hyperemia) that causes redness (erythema) and the entry of fluid into the tissues (edema).

The main features of the vascular phase of the inflammatory response are vasodilation, i.e. widening of the blood vessels to increase the blood flow to the infected area; increased vascular permeability which allows diffusible components to enter the site; cellular infiltration by chemotaxis; or the directed movement of inflammatory cells, including neutrophils, through the walls of blood vessels into the site of injury; changes in biosynthetic, metabolic, and catabolic profiles of many organs; and activation of cells of the immune system as well as of complex enzymatic systems of blood plasma. This leads to the cellular phase where neutrophils are attracted to the site of injury by the presence of chemotaxins neutrophils then recognise the foreign body and begin phagocytosis. Inflammation which runs unchecked can, however lead to a host of diseases including acute hepatitis, acute pancreatitis, acute kidney disease, inflammatory bowel disease, inflammatory liver diseases, rheumatoid arthritis, autoimmunity, sepsis, SIRS, and atherosclerosis.

The acute vascular response can be followed by an acute cellular response which takes place over the next few hours. The hallmark of this phase is the appearance of granulocytes, particularly neutrophils, in the tissues. These cells first attach themselves to the endothelial cells within the blood vessels (margination) and then cross into the surrounding tissue (diapedesis). During this phase erythrocytes may also leak into the tissues and a hemorrhage can occur. If the vessel is damaged, fibrinogen and fibronectin are deposited at the site of injury, platelets aggregate and become activated, and the red cells stack together in what are called “rouleau” to help stop bleeding and aid clot formation. The dead and dying cells contribute to pus formation. If the damage is sufficiently severe, a chronic cellular response may follow over the next few days. A characteristic of this phase of inflammation is the appearance of a mononuclear cell infiltrate composed of macrophages and lymphocytes. The macrophages are involved in microbial killing, in clearing up cellular and tissue debris, and in remodeling of tissues.

### SUMMARY OF THE DISCLOSURE

The present disclosure relates to a method of treating an acute inflammatory condition in a subject comprising administering to the subject a therapeutically effective amount of a compound represented by Formula (I):

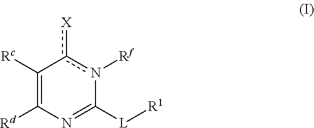

or a pharmaceutically acceptable salt or tautomer thereof,
wherein:
X is O or OH;
L is —(CH₂)ₘCH₂CH₂—, —(CH₂)ₘY(CH₂)ₚ—, —(CH₂)ₘC(O)(CH₂)ₚ—, —(CH₂)ₘC(O)O(CH₂)ₚ—, —(CH₂)ₘC(O)NR²(CH₂)ₚ—, or —(CH₂)ₘNR²C(O)(CH₂)ₚ;
Y is O, N or S(O)_{q};
R¹ is C₆-C₁₀ aryl or heteroaryl, wherein the aryl and heteroaryl are substituted with R^{a} and R^{b}, and optionally substituted with one or more R^{e};
R² is H or C₁-C₆ alkyl;
one of R^{a} and R^{b} is hydrogen and the other is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl;
R^{c} is H, C₁-C₆ alkyl, C₁-C₆ haloalkyl, halogen, —CN, —OR^{x}, —CO₂R^{x}, or NO₂;
R^{d} is methyl, optionally substituted 5- to 10-membered aryl, optionally substituted 5- or 6-membered heteroaryl, or optionally substituted 5- or 6-membered carbocycle;
each R^{x} is independently at each occurrence hydrogen or C₁-C₆ alkyl;
each R^{e} is independently C₁-C₆ alkyl, C₂-C₆ alkenyl, C₂-C₆ alkynyl, halogen, —OR^{y}, C₁-C₆ haloalkyl, —NHR^{z}, —OH, or —CN;
R^{f} is H or absent;
each R^{y} and R^{z} is independently hydrogen, C₁-C₆ alkyl, or C₁-C₆ haloalkyl;
each m and p independently is 0, 1, or 2, wherein m+p<3;
q is 0, 1, or 2;
r is 0 or 1; and
the dotted line is an optional double bond;
with the proviso that R^{c} is not hydrogen or —CN when X is O, L is —SCH₂— and R^{d} is optionally substituted phenyl, R^{c} is not C₁-C₆ alkyl when X is O, L is —SCH₂— and R^{d} is methyl, and that R^{c} is not —CN when X is O, L is —SCH₂— and R^{d} is 2-furyl.

The present disclosure relates to treating an acute inflammatory condition comprising administering to a subject in need thereof, a therapeutically effective amount of a compound, or a pharmaceutically acceptable salt or tautomer thereof, selected from the group consisting of

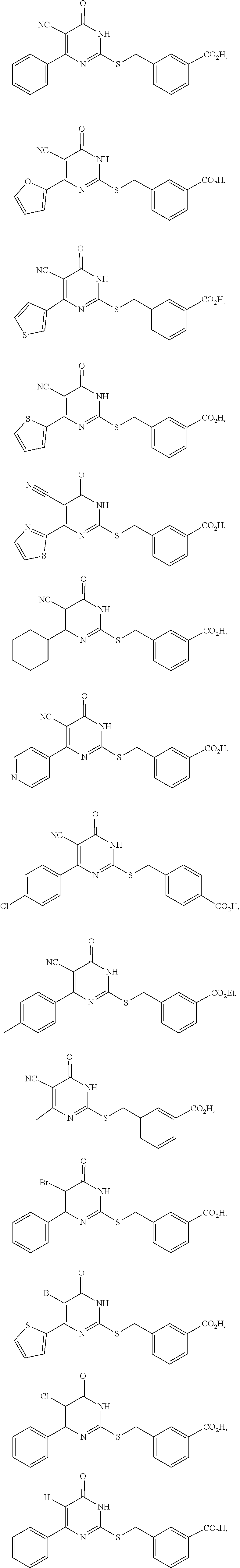

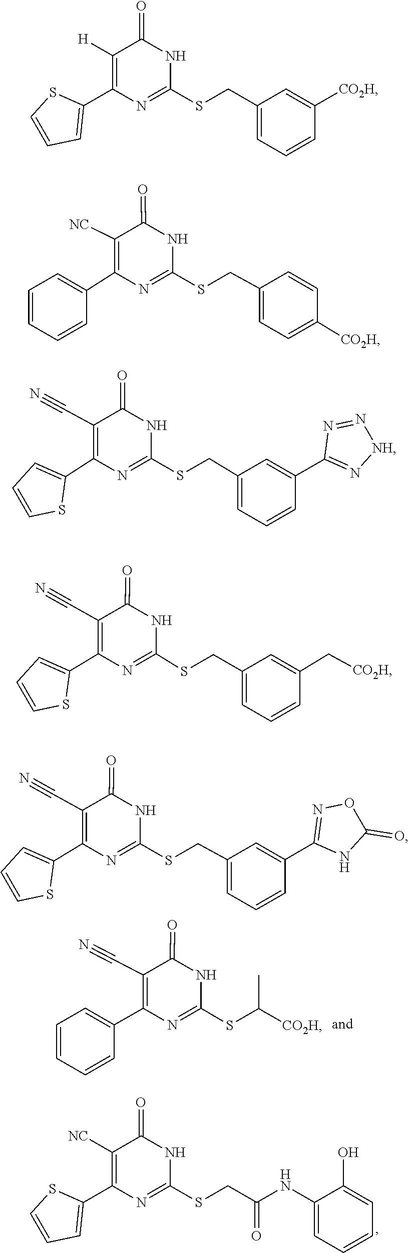

or a pharmaceutically acceptable salt thereof.

The present disclosure relates to treating an acute inflammatory condition comprising administering to a subject in need thereof, a therapeutically effective amount of a compound, or a pharmaceutically acceptable salt or tautomer thereof, selected from the group consisting of

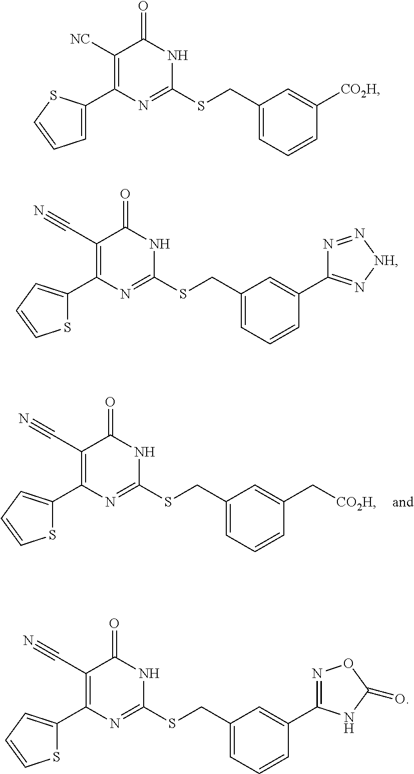 [SMILES: N#Cc1c(-c2cccs2)nc(SCc2cccc(C(=O)O)c2)[nH]c1=O ; N#Cc1c(-c2cccs2)nc(SCc2cccc(-c3nn[nH]n3)c2)[nH]c1=O ; N#Cc1c(-c2cccs2)nc(SCc2cccc(CC(=O)O)c2)[nH]c1=O ; N#Cc1c(-c2cccs2)nc(SCc2cccc(-c3noc(=O)[nH]3)c2)[nH]c1=O]

The present disclosure relates to treating an acute inflammatory condition comprising administering to a subject in need thereof, a therapeutically effective amount of a compound, or a pharmaceutically acceptable salt or tautomer thereof, selected from the group consisting of

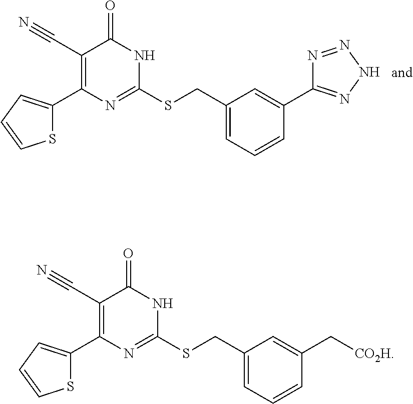 [SMILES: N#Cc1c(-c2cccs2)nc(SCc2cccc(-c3nn[nH]n3)c2)[nH]c1=O ; N#Cc1c(-c2cccs2)nc(SCc2cccc(CC(=O)O)c2)[nH]c1=O]

The present disclosure relates to treating an acute inflammatory condition comprising administering to a subject in need thereof, a therapeutically effective amount of a compound, or a pharmaceutically acceptable salt or tautomer thereof, of formula:

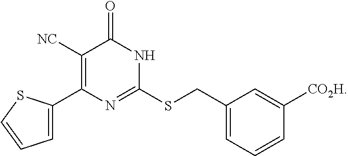 [SMILES: N#Cc1c(-c2cccs2)nc(SCc2cccc(C(=O)O)c2)[nH]c1=O]

The present disclosure relates to treating an acute inflammatory condition comprising administering to a subject in need thereof, a therapeutically effective amount of a compound, or a pharmaceutically acceptable salt or tautomer thereof, of formula:

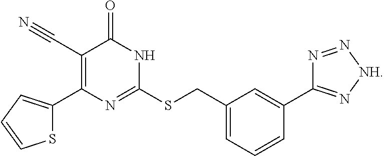 [SMILES: N#Cc1c(-c2cccs2)nc(SCc2cccc(-c3nn[nH]n3)c2)[nH]c1=O]

The present disclosure relates to treating an acute inflammatory condition comprising administering to a subject in need thereof, a therapeutically effective amount of a compound, or a pharmaceutically acceptable salt or tautomer thereof, of formula:

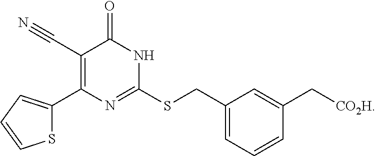 [SMILES: N#Cc1c(-c2cccs2)nc(SCc2cccc(CC(=O)O)c2)[nH]c1=O]

The present disclosure relates to treating an acute inflammatory condition comprising administering to a subject in need thereof, a therapeutically effective amount of a compound, or a pharmaceutically acceptable salt or tautomer thereof, of formula:

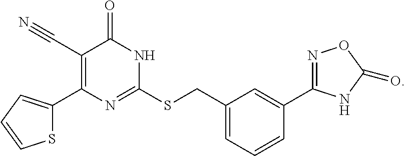 [SMILES: N#Cc1c(-c2cccs2)nc(SCc2cccc(-c3noc(=O)[nH]3)c2)[nH]c1=O]

In further aspects, provided herein is a pharmaceutical composition, comprising a compound as described herein, or a pharmaceutically acceptable salt or tautomer thereof, and a pharmaceutically acceptable excipient. The present disclosure relates to treating an acute inflammatory condition comprising administering to a subject in need thereof, the pharmaceutical composition of the present disclosure.

The present disclosure provides a compound of the present disclosure, and pharmaceutically acceptable salts or tautomers thereof, or the pharmaceutical composition of the present disclosure for use in the treatment of an acute inflammatory condition in a subject in need thereof.

The present disclosure provides for use of a compound of the present disclosure, and pharmaceutically acceptable salts or tautomers thereof, for the treatment of an acute inflammatory condition in a subject in need thereof.

The present disclosure provides for use of a compound of the present disclosure, and pharmaceutically acceptable salts or tautomers thereof, in the manufacture of a medicament for the treatment of an acute inflammatory condition.

### BRIEF DESCRIPTION OF THE DRAWINGS

FIG. 1 shows secretion of the pro-inflammatory cytokines; tumour necrosis factor α, TNFα, interleukins, IL-1α, IL-1β, and IL-6 in macrophages stimulated with LPS is reduced by short hairpin RNA, siACMSD, with respect to vehicle controls.

FIG. 2 shows expression of ACMSD and QPRT under inflammatory conditions.

FIG. 3 shows expression of QPRT in human primary bone marrow mononuclear cells (BMMCs).

FIG. 4 shows inhibition of ACMSD with compound I-17 and compound I-18, restores NAD⁺ biosynthesis in Kupffer cells, primary hepatocytes, and kidney proximal tubular HK2 cells.

FIG. 5 shows that ACMSD inhibition with compound I-18 increases expression of both of the downstream target genes sirtuin, SIRT-1 in Kupffer cells and SIRT-3 in HK2 primary tubule cells.

FIG. 6 shows that both compounds I-17 and I-18 (at 500 nM) activate SIRT1 in primary hepatocytes measured at 24 hrs after treatment.

FIG. 7 shows inhibition of ACMSD with compound I-18 (50βM) also reverses the reduction of SIRT expression induced by a Cisplatin insult in HK2 primary tubule cells.

FIG. 8 shows that SIRT1 is a central component of the SIRT1/STAT3 pathway whereby increased expression and activity of SIRT1, induced by ACMSD inhibition with compound I-18, increases expression of signal transducer and activator of transport 3, STAT3, in Kupffer cells, in a dose-dependent manner.

FIG. 9 shows that ACMSD inhibition with compound I-18 increases expression of IL-10, in Kupffer cells, in a dose-dependent manner to thus promote an anti-inflammatory response.

FIG. 10 shows that the secretion of the pro-inflammatory cytokine, IL-6 is reduced by inhibition of ACMSD with compounds I-17 and I-18 (1 μM), and in a dose-dependent manner with compound I-18 in Kupffer cells following LPS-induced inflammation.

FIG. 11 shows that the secretion of the pro-inflammatory cytokine, IL-6 is also reduced in a dose-dependent manner by inhibition of ACMSD with compounds I-17 and I-18 in a co-culture primary hepatocytes and Kupffer cells following LPS-induced inflammation (50 ng/mL).

FIG. 12 shows inhibition of ACMSD with compound I-18 (5μM), also reduces the secretion of LPS-induced IL-6 in human primary bone marrow mononuclear cells (BMMCs).

FIG. 13 shows that inhibition of ACMSD, with compounds I-17 and I-18 in Kupffer cells, BMMC cells, and in a co-culture of primary hepatocytes and stellate cells, reduces the secretion of the inflammatory cytokine TNFα following LPS-induced inflammation (50 ng/mL).

FIG. 14 shows that the expression of TNFα is reduced by inhibition of ACMSD with both compounds I-17 and I-18 (5 μM) in a co-culture of primary hepatocytes and stellate cells following treatment with a free fatty acid mix (after 6 hr exposure to a mixture of palmitate (0.33 mM) and oleate (0.66 mM)) and in ex-vivo kidney tissue samples from a CLP-induced sepsis model in mice following an IP 30 mg/kg/dose of compound I-18.

FIG. 15 shows that inhibition of ACMSD, with compounds I-17 and I-18 (1 μM) in Kupffer cells and in a co-culture of primary hepatocytes and stellate cells, reduces the secretion of the inflammatory cytokine IL-1β following LPS-induced inflammation (50 ng/mL).

FIG. 16 shows that the expression of IL-1β is reduced by inhibition of ACMSD with compound I-17 (5 μM) in a co-culture of primary hepatocytes and stellate cells following treatment with a free fatty acid mix (after 6 hr exposure to a mixture of palmitate (0.33 mM) and oleate (0.66 mM)) and in ex-vivo kidney tissue samples from a CLP-induced sepsis model in mice, following an IP 30 mg/kg/dose of compound I-18.

FIG. 17 shows that the expression of MCP1 is reduced by inhibition of ACMSD in ex-vivo kidney tissue samples from a CLP-induced sepsis model in mice, following an IP 30 mg/kg/dose of compound I-18.

FIG. 18 shows that the inhibition of ACMSD with compound I-17 or compound I-18 (5 μM), reduces the expression of pro-fibrotic cytokine, TGF-β, the pro-fibrotic chemokines, CTGF, and the pro-fibrotic genes, Bcl-2-associated gene X, BAX, actin alpha 2, ACTA2, collagen type 1 alpha 1 chain, Col1A1, fibronectin, thrombospondin-1, THBS-1, and tissue inhibitor of metalloprotinases 2, TIMP2, in a co-culture of primary hepatocytes and stellate cells following treatment with a free fatty acid mix (after 6 hr exposure to a mixture of palmitate (0.33 mM) and oleate (0.66 mM).

FIG. 19 shows that the anti-fibrotic effect of ACMSD inhibition via the reduction of transforming growth factor beta, TGF-β is mediated by the TGFβ/SMAD pathway as also shown by the dose-dependent reduction of mothers against decapentaplegic homologue 3, SMAD3, in both preventive and therapeutic treatment with compound I-18 following TGF-β-induced fibrosis in HK2 cells.

FIG. 20 shows that the anti-fibrotic effect of ACMSD inhibition is further demonstrated by a dose-dependent reduction in the expression of fibronectin and TIMP2 by compound I-18.

FIG. 21 shows, in mouse primary hepatocytes, the inhibition of ACMSD with either compound I-17 or I-18, increases the activity of mitochondrial superoxide dismutase 2, SOD2, in a dose-dependent manner, after treatment for 24 hrs.

FIG. 22 shows, in HK-2 cells, ACMSD inhibition with compound I-18 (100 μM) also increases the expression of both SOD2 and mitochondrial dynamin-like 120 kDa protein, OPA-1, following cisplatin-insult.

FIG. 23 shows that ACMSD inhibition with compounds I-17, (in various cell types) modulates cellular ROS production and mitochondrial biogenesis by increasing the expression of mitochondrial transcription factor A, TFAM in primary hepatocytes, via SIRT-1 activation, and OPA-1 a mitochondrial fusion protein in rat renal NRK52E cells, via SIRT-3 activation following Cisplatin insult (100 μM).

FIG. 24 shows, in primary hepatocytes, inhibition of ACMSD with compound I-17 after 6 hr exposure to a mixture of palmitate (0.33 mM) and oleate (0.66 mM) increases the mRNA levels of fatty acid oxidation genes; medium-chain acyl-CoA dehydrogenase, Mcad, carnitine palmitoyltransferase 1, Cpt1α, hydroxyacyl-CoA dehydrogenase, Hadha1, hormone sensitive lipase, Hsl, pyruvate dehydrogenase lipoamide kinase 4, Pdk4, and succinate dehydrogenase, Sdha.

FIG. 25 shows that ACMSD inhibition with compound I-18 at 100 μM increases the mRNA levels of mitochondrial and oxidative stress genes; citrate synthase, CS; NDAH ubiquinone oxidoreductase subunit 2, Ndufa2, cytochrome c oxidase subunit 2, Cox2, ATP synthase lipid-binding protein, Atp5g1, superoxide dismutase 1, Sod1, and superoxide dismutase 2, Sod2.

FIG. 26 shows that ACMSD inhibition with compounds I-17 and I-18, increases the mRNA levels of mitochondrial genes, TFAM, Ndufa, ubiquinol-cytochrome C reductase core protein 1, Uqcrc1, CytC, Atp5g1, and CS in mouse primary hepatocytes after 24 hr treatment.

FIG. 27 shows that the increase in mRNA levels of mitochondrial genes, Sod1, Ndufa2, Cox2, and CytC, in HK-2 cells after 24 hr treatment with the ACMSD inhibitor, compound I-18, is SIRT1 dependent and is blocked by the inhibition of Sirt1 as shown by treatment with Compound I-18 in combination with the SIRT1 inhibitor, EX527, at the indicated concentrations.

FIG. 28 shows that administration of ACMSD inhibitors, compounds I-17 and I-18, also induces the transcription of the mitochondrial genes; medium-chain acyl-CoA dehydrogenase, Mcad, carnitine palmitoyltransferase 1, Cpt1a, NDAH ubiquinone oxidoreductase subunit 2, Ndufa2, ATP synthase lipid-binding protein, Atp5g1, and superoxide dismutase 2, Sod2 in the liver, whereas the expression of the same genes in the kidneys was unaffected.

FIG. 29 shows that inhibition of ACMSD with compound I-17 in primary hepatocytes increases the expression of mitochondrial SOD2.

FIG. 30 shows that murine Kupffer cells treated with LPS and Compound I-18 show a decrease in the M1 phenotype gene markers, iNOS, IL-6, and TNFα.

FIG. 31 shows corresponding increase in the M2 phenotype gene markers, Arginase-1, Mannose Receptor (Mrc-2), and IL-10.

FIG. 32 shows two different stimuli (LPS and IL-4) performed to evaluate M2 phenotype.

### DETAILED DESCRIPTION OF THE DISCLOSURE

The following description sets forth numerous exemplary configurations, methods, parameters, and the like. It should be recognized, however, that such description is not intended as a limitation on the scope of the present disclosure, but is instead provided as a description of exemplary embodiments.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar to or equivalent to those described herein can be used in the practice and testing of the disclosure, suitable methods and materials are described below. All publications, patent applications, patents, and other references mentioned herein are incorporated by reference. The references cited herein are not admitted to be prior art to the claimed disclosure. In the case of conflict, the present specification, including definitions, will control. In addition, the materials, methods, and examples are illustrative only and not intended to be limiting.

Other features and advantages of the disclosure will be apparent from the following detailed description and claims.

The present disclosure relates to a method of treating an acute inflammatory condition in a subject comprising administering to the subject a therapeutically effective amount of a pyrimidinone compound.

### Compounds

The present disclosure relates to compounds of Formula (I):

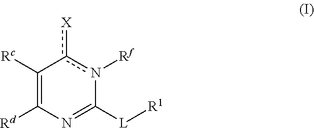

or a pharmaceutically acceptable salt or tautomer thereof,
wherein:
X is O or OH;
L is —(CH₂)ₘCH₂CH₂—, —(CH₂)ₘY(CH₂)ₚ—, —(CH₂)ₘC(O)(CH₂)ₚ—, —(CH₂)ₘC(O)O(CH₂)ₚ—, —(CH₂)ₘC(O)NR²(CH₂)ₚ—, or —(CH₂)ₘNR²C(O)(CH₂)ₚ;
Y is O, N, or S(O)_{q};
R¹ is C₆-C₁₀ aryl or heteroaryl, wherein the aryl and heteroaryl are substituted with R^{a} and R^{b}, and optionally substituted with one or more R^{e};
R² is H or C₁-C₆ alkyl;
one of R^{a} and R^{b} is hydrogen and the other is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl;
R^{c} is H, C₁-C₆ alkyl, C₁-C₆ haloalkyl, halogen, —CN, —OR^{x}, —CO₂R^{x}, or NO₂;
R^{d} is methyl, optionally substituted 5- to 10-membered aryl, optionally substituted 5- or 6-membered heteroaryl, or optionally substituted 5- or 6-membered carbocycle;
each R^{x} is independently at each occurrence hydrogen or C₁-C₆ alkyl;
each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN;
R^{f} is H or absent;
each R^{y} and R^{z} is independently hydrogen, C₁-C₆ alkyl, or C₁-C₆ haloalkyl;
each m and p independently is 0, 1, or 2, wherein m+p<3;
q is 0, 1, or 2;
r is 0 or 1; and
the dotted line is an optional double bond;
with the proviso that R^{c} is not hydrogen or —CN when X is O, L is —SCH₂— and R^{d} is optionally substituted phenyl, R^{c} is not C₁-C₆ alkyl when X is O, L is —SCH₂— and R^{d} is methyl, and that R is not —CN when L is —SCH₂— and R^{d} is 2-furyl.

In certain embodiments, the present disclosure relates to compounds of Formula (I):

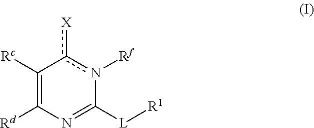

or a pharmaceutically acceptable salt or tautomer thereof,
wherein:
X is O or OH;
L is —(CH₂)ₘCH₂CH₂—, —(CH₂)ₘY(CH₂)ₚ—, —(CH₂)ₘC(O)(CH₂)ₚ—, —(CH₂)ₘC(O)O(CH₂)ₚ—, —(CH₂)ₘC(O)NR²(CH₂)ₚ—, or —(CH₂)ₘNR²C(O)(CH₂)ₚ;
Y is O, N, or S(O)q;
R¹ is C₆-C₁₀ aryl or heteroaryl, wherein the aryl and heteroaryl are substituted with R^{a} and R^{b}, and optionally substituted with one or more R^{e};
R² is H or C₁-C₆ alkyl;
one of R^{a} and R is hydrogen and the other is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣdihydrotetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl;
R^{c} is H, C₁-C₆ alkyl, C₁-C₆ haloalkyl, halogen, —CN, —OR^{x}, —CO₂R^{x}, or NO₂;
R^{d} is methyl, optionally substituted 5- to 10-membered aryl, optionally substituted 5- or 6-membered heteroaryl, or optionally substituted 5- or 6-membered carbocycle;
each R^{x} is independently at each occurrence hydrogen or C₁-C₆ alkyl;
each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN;
R^{f} is H or absent;
each R^{y} and R^{z} is independently hydrogen, C₁-C₆ alkyl, or C₁-C₆ haloalkyl;
each m and p independently is 0, 1, or 2, wherein m+p<3;
q is 0, 1, or 2;
r is 0 or 1; and
the dotted line is an optional double bond;
with the proviso that R^{c} is not hydrogen or —CN when X is O, L is —SCH₂— and R^{d} is optionally substituted phenyl, R^{c} is not C₁-C₆ alkyl when X is O, L is —SCH₂— and R^{d} is methyl, and that R is not —CN when L is —SCH₂— and R^{d} is 2-furyl.

In some embodiments of Formula (I), X is O or OH. In other embodiments, X is O. In other embodiments, X is OH.

In some embodiments of Formula (I), L is —(CH₂)ₘCH₂CH₂—, —(CH₂)ₘY(CH₂)ₚ—, —(CH₂)ₘC(O)(CH₂)ₚ—, —(CH₂)ₘC(O)O(CH₂)ₚ—, —(CH₂)ₘC(O)NR²(CH₂)ₚ—, or —(CH₂)ₘNR²C(O) (CH₂)ₚ. In other embodiments, L is —CH₂CH₂—, —CH₂CH₂CH₂—, —SCH₂—, —SCH₂CH₂—, —CH₂S—, —CH₂SCH₂—, —CH₂CH₂S—, —S(O)CH₂—, —S(O)CH₂CH₂—, —CH₂S(O)—, —CH₂S(O)CH₂—, —CH₂CH₂S(O)—, —S(O)₂CH₂—, —S(O)₂CH₂CH₂—, —CH₂S(O)₂—, —CH₂S(O)₂CH₂—, —CH₂—, —CH₂S(O)₂—, —OCH₂—, —OCH₂CH₂—, —CH₂O—, —CH₂OCH₂—, —CH₂CH₂O—, —NR²CH₂—, —CH₂NR²—, —CH₂NR²CH₂—, —CH₂CH₂NR²—, —NR²CH₂CH₂—, —C(O)CH₂—, —C(O)CH₂CH₂—, —C(O)O—, —C(O)OCH₂—, —CH₂C(O)O—, —C(O)NR²—, —C(O)NR²CH₂—, —NR²C(O), —NR²C(O)CH₂, or —CH₂NR²C(O). In other embodiments, L is —CH₂CH₂—, —CH₂CH₂CH₂—, —SCH₂—, —SCH₂CH₂—, —S(O)CH₂—, —S(O)CH₂CH₂—, —S(O)₂CH₂—, —S(O)₂CH₂CH₂—, —OCH₂—, —OCH₂CH₂—, —NR²CH₂—, —NR²CH₂CH₂—, —C(O)CH₂—, —C(O)CH₂CH₂—, —C(O)O—, —C(O)OCH₂—,—CH₂C(O)O—, —C(O)NR²—, —C(O)NR²CH₂—, —NR²C(O), or —NR²C(O)CH₂. In other embodiments, L is —CH₂CH₂—, —CH₂C(O)—, —C(O)CH₂—, —NR²CH₂—, —CH₂N^{R2}—, —OCH₂—, —CH₂O—, —SCH₂—, —CH₂S—, —S(O)CH₂—, —CH₂S(O)—, —CH₂S(O)₂—, or —S(O)₂CH₂—.

In some embodiments of Formula (I), R¹ is C₆-C₁₀ aryl or heteroaryl, wherein the aryl and heteroaryl are substituted with R^{a} and R^{b}, and optionally substituted with one or more R^{e}. In other embodiments, R¹ is C₆-C₁₀ aryl substituted with R^{a} and R^{b}, and optionally substituted with one or more R^{e}. In other embodiments, R¹ is heteroaryl substituted with R^{a} and R^{b}, and optionally substituted with one or more R^{e}. In further embodiments, R¹ is phenyl substituted with R^{a} and R^{b}, and optionally substituted with one or more R^{e}.

In some embodiments of Formula (I), R^{a} is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl. In some embodiments of Formula (I), R^{a} is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣdihydrotetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl. In other embodiments, R^{a} is —(CH₂)ᵣCO₂R^{x}, —OCH₂CO₂R^{x}, tetrazole, —(CH₂)tetrazole, oxadiazolone, —(CH₂)oxadiazolone, tetrazolone, —(CH₂)tetrazolone, thiadiazolol, —(CH₂)thiadiazolol, isoxazol-3-ol, —(CH₂) isoxazol-3-ol, —P(O)(OH)OR^{x}, —(CH₂)P(O)(OH)OR^{x}, —S(O)₂OH, —(CH₂)S(O)₂OH, —C(O)NHCN, —(CH₂)C(O)NHCN, —C(O)NHS(O)₂alkyl, or —(CH₂)C(O)NHS(O)₂alkyl. In other embodiments, R^{a} is hydrogen, CO₂R^{x}, CH₂CO₂R^{x}, tetrazole, or oxadiazolone. In further embodiments, R^{a} is hydrogen, CO₂H, CH₂CO₂H, tetrazole, or 1,2,4-oxadiazol-5(4H)-one.

In some embodiments of Formula (I), R^{b} is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl. In other embodiments, R^{b} is —(CH₂)ᵣCO₂R^{x}, —OCH₂CO₂R^{x}, tetrazole, —(CH₂)tetrazole, oxadiazolone, —(CH₂)oxadiazolone, tetrazolone, —(CH₂)tetrazolone, thiadiazolol, —(CH₂)thiadiazolol, isoxazol-3-ol, —(CH₂) isoxazol-3-ol, —P(O)(OH)OR^{x}, —(CH₂)P(O)(OH)OR^{x}, —S(O)₂OH, —(CH₂)S(O)₂OH, —C(O)NHCN, —(CH₂)C(O)NHCN, —C(O)NHS(O)₂alkyl, or —(CH₂)C(O)NHS(O)₂alkyl. In other embodiments, R^{b} is hydrogen, CO₂R^{x}, CH₂CO₂R^{x}, tetrazole, or oxadiazolone. In further embodiments, R^{b} is hydrogen, CO₂H, CH₂CO₂H, tetrazole, or 1,2,4-oxadiazol-5(4H)-one. In further embodiments, R^{b} is hydrogen.

In some embodiments of Formula (I), R^{c} is H, C₁-C₆ alkyl, C₁-C₆ haloalkyl, halogen, —CN, —OR^{x}, —CO₂R^{x}, or NO₂. In other embodiments, R^{c} is C₁-C₆ alkyl, C₁-C₆ haloalkyl, halogen, —CN, —OR^{x}, —CO₂R^{x}, or NO₂. In other embodiments, R^{c} is halogen, —CN, —OR^{x}, or C₁-C₆ alkyl. In other embodiments, R^{c} is halogen, —CN, —OR^{x}, or C₁-C₃ alkyl. In other embodiments, R^{c} is H, —CN, or halogen. In other embodiments, R^{c} is —CN or halogen.

In some embodiments of Formula (I), R^{d} is methyl, optionally substituted 5- to 10-membered aryl, optionally substituted 5- or 6-membered heteroaryl, or optionally substituted 5- or 6-membered carbocycle. In other embodiments, R^{d} is methyl, optionally cyclohexyl, optionally substituted pyridinyl, optionally substituted thiazolyl, optionally substituted phenyl, or optionally substituted thienyl. In other embodiments, R^{d} is methyl, cyclohexyl, pyridinyl, thiazolyl, phenyl, or thienyl. In other embodiments, R^{d} is cyclohexyl, pyridinyl, thiazolyl, phenyl, or thienyl, wherein each is optionally substituted with one or more substituents independently selected from halogen, C₁-C₆ alkyl, C₁-C₆ hydroxyalkyl, C₁-C₆ alkoxy, C₁-C₆ haloalkyl, C₁-C₆ haloalkoxy, —OH, CN, and amino. In other embodiments, R^{d} is cyclohexyl, pyridinyl, thiazolyl, phenyl, or thienyl, wherein each is optionally substituted with one or more substituents independently selected from halogen, C₁-C₆ alkyl, C₁-C₆ hydroxyalkyl, C₁-C₆ alkoxy, C₁-C₆ haloalkyl, and C₁-C₆ haloalkoxy. In other embodiments, R^{d} is cyclohexyl, pyridinyl, thiazolyl, phenyl, or thienyl, wherein each is optionally substituted with one or more halogen. In yet other embodiments, R^{d} is cyclohexyl, pyridinyl, thiazolyl, phenyl, or thienyl. In other embodiments, R^{d} is cyclohexyl, pyridinyl, thiazolyl, phenyl, 4-chlorophenyl, 4-methylphenyl, or thienyl.

In some embodiments of Formula (I), each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN. In other embodiments, C₁-C₄ alkyl, C₂-C₄ alkenyl, C₂-C₄ alkynyl, halogen, —OR^{Y}, C₁-C₄ haloalkyl, —NHR^{z}, —OH, or —CN.

In some embodiments of Formula (I), R is H or absent. In other embodiments, R is H. In other embodiments, R is absent, when N to which it is attached participates in a double bond.

In some embodiments of Formula (I), R^{x} is hydrogen or C₁-C₆ alkyl. In other embodiments, R^{x} is hydrogen or C₁-C₃ alkyl. In further embodiments, R^{x} is hydrogen, methyl, ethyl, n-propyl, or iso-propyl.

In some embodiments of Formula (I), R^{y} is independently hydrogen, C₁-C₆ alkyl, or C₁-C₆ haloalkyl. In other embodiments, R^{y} is hydrogen, C₁-C₃ alkyl, or C₁-C₃ haloalkyl.

In some embodiments of Formula (I), each R^{z} is independently hydrogen, C₁-C₆ alkyl, or C₁-C₆ haloalkyl. In other embodiments, R^{z} is hydrogen, C₁-C₃ alkyl, or C₁-C₃ haloalkyl.

In some embodiments of Formula (I), m is 0, 1 or 2. In other embodiments, m is 0. In other embodiments, m is 1. In yet other embodiments, m is 2.

In some embodiments of Formula (I), p is 0, 1 or 2. In other embodiments, p is 0. In other embodiments, p is 1. In yet other embodiments, p is 2.

In some embodiments of Formula (I), m+p<3.

In some embodiments of Formula (I), q is 0, 1, or 2. In other embodiments, q is 0. In other embodiments, q is 1. In other embodiments, q is 2.

In some embodiments of Formula (I), r is 0 or 1. In other embodiments, r is 0. In other embodiments, r is 1.

In some embodiments of Formula (I), the dotted line is a single bond. In other embodiments, the dotted line is a double bond.

In some embodiments of Formula (I), one of R^{a} and R^{b} is hydrogen and the other is CO₂R^{x}, CH₂CO₂R^{x}, tetrazole, or oxadiazolone. In other embodiments, R^{b} is hydrogen and R^{a} is CH₂CO₂H, tetrazole, or (1,2,4-oxadiazol-5(4H)-one).

In some embodiments of Formula (I), R^{b} is hydrogen, R^{c} is —CN, R^{d} is thienyl, and R^{a} is CH₂CO₂H, tetrazole, or (1,2,4-oxadiazol-5(4H)-one).

In some embodiments of Formula (I), R^{c} is halogen, R^{a} is —CO₂H, and R^{b} is H. In other embodiments, R^{c} is —Br, R^{a} is —CO₂H, and R^{b} is H. In further embodiments, R^{c} is —Cl, R^{a} is —CO₂H, and R^{b} is H.

In some embodiments of Formula (I), R^{c} is halogen, R^{a} is tetrazole, and R^{b} is H. In other embodiments, R^{c} is —Br, R^{a} is tetrazole, and R^{b} is H. In further embodiments, R^{c} is —Cl, R^{a} is tetrazole, and R^{b} is H.

In some embodiments of Formula (I), R^{c} is halogen, R^{a} is —CH₂CO₂H, and R^{b} is H. In other embodiments, R^{c} is —Br, R^{a} is —CH₂CO₂H, and R^{b} is H. In further embodiments, R^{c} is —Cl, R^{a} is —CH₂CO₂H, and R^{b} is H.

In some embodiments of Formula (I), R^{c} is halogen, R^{a} is (1,2,4-oxadiazol-5(4H)-one), and R^{b} is H. In other embodiments, R^{c} is —Br, R^{a} is (1,2,4-oxadiazol-5(4H)-one), and R^{b} is H. In other embodiments, R^{c} is —Cl, R^{a} is (1,2,4-oxadiazol-5(4H)-one), and R^{b} is H.

In some embodiments of Formula (I), R^{c} is —CN, R^{a} is —CO₂H, and R^{b} is H. In other embodiments, R^{c} is —CN, R^{a} is —CH₂CO₂H, and R^{b} is H. In other embodiments, R is —CN, R^{a} is tetrazole, and R^{b} is H. In yet other embodiments, R^{c} is —CN, R^{a} is (1,2,4-oxadiazol-5(4H)-one), and R^{b} is H.

In some embodiments of Formula (I), R^{c} is not hydrogen or —CN and X is O, L is —SCH₂— and R^{d} is optionally substituted phenyl. In other embodiments, R^{c} is not C₁-C₆ alkyl and X is O, L is —SCH₂— and R^{d} is methyl. In other embodiments, R^{c} is not —CN and X is O, L is —SCH₂— and R^{d} is 2-furyl.

In some embodiments of Formula (I), R^{c} is not hydrogen or —CN when X is O, L is —SCH₂— and R^{d} is optionally substituted phenyl.

In some embodiments of Formula (I), R^{c} is not C₁-C₆ alkyl when X is O, L is —SCH₂— and R^{d} is methyl.

In some embodiments of Formula (I), R^{c} is not —CN when X is O, L is —SCH₂— and R^{d} is 2-furyl.

In one embodiment, the compound of Formula (I) is represented by Formula (Ia):

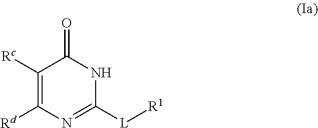

or a pharmaceutically acceptable salt, or tautomer thereof,
wherein:
L is —(CH₂)ₘCH₂CH₂—, —(CH₂)ₘY(CH₂)ₚ—, —(CH₂)ₘC(O)(CH₂)ₚ—, —(CH₂)ₘC(O)O(CH₂)ₚ—, —(CH₂)ₘC(O)NR²(CH₂)ₚ—, or —(CH₂)ₘNR²C(O)(CH₂)ₚ;
Y is O, N or S(O)q;
R¹ is C₆-C₁₀ aryl or heteroaryl, wherein the aryl and heteroaryl are substituted with R^{a} and R^{b}, and optionally substituted with one or more R^{e};
R² is H or C₁-C₆ alkyl;
one of R^{a} and R^{b} is hydrogen and the other is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl;
R^{c} is H, C₁-C₆ alkyl, C₁-C₆ haloalkyl, halogen, —CN, —OR^{x}, —CO₂R^{x}, or NO₂;
R^{d} is methyl, optionally substituted 5- to 10-membered aryl, optionally substituted 5- or 6-membered heteroaryl, or optionally substituted 5- or 6-membered carbocycle;
each R^{x} is independently at each occurrence hydrogen or C₁-C₆ alkyl;
each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN;
each R^{y} and R^{z} is independently hydrogen, C₁-C₆ alkyl, or C₁-C₆ haloalkyl;
each m and p independently is 0, 1 or 2, wherein m+p<3;
q is 0, 1, or 2; and
r is 0 or 1;
with the proviso that R^{c} is not hydrogen or —CN when L is —SCH₂— and R^{d} is optionally substituted phenyl, R^{c} is not C₁-C₆ alkyl when L is —SCH₂— and R^{d} is methyl, and that R is not —CN when L is —SCH₂— and R^{d} is 2-furyl.

In some embodiments of Formula (Ia),
L is —CH₂CH₂—, —CH₂C(O)—, —C(O)CH₂—, —NR²CH₂—, —CH₂NR²—, —OCH₂—, —CH₂O—, —SCH₂—, —CH₂S—, —S(O)CH₂—, —CH₂S(O)—, —CH₂S(O)₂—, or —S(O)₂CH₂—;
Y is O, N or S(O)q;
R¹ is C₆-C₁₀ aryl or heteroaryl, wherein the aryl and heteroaryl are substituted with R^{a} and R^{b}, and optionally substituted with one or more R^{e};
R² is H or C₁-C₆ alkyl;
one of R^{a} and R^{b} is hydrogen and the other is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣ C(O)NHS(O)₂alkyl;
R^{c} is C₁-C₆ alkyl, C₁-C₆ haloalkyl, halogen, —CN, —OR^{x}, —CO₂R^{x}, or NO₂;
R^{d} is methyl, optionally substituted 5- to 10-membered aryl, optionally substituted 5- or 6-membered heteroaryl, or optionally substituted 5- or 6-membered carbocycle;
each R^{x} is independently at each occurrence hydrogen or C₁-C₆ alkyl;
each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN;
each R^{y} and R^{z} is independently hydrogen, C₁-C₆ alkyl, or C₁-C₆ haloalkyl;
each m and p independently is 0, 1, or 2, wherein m+p<3;
q is 0, 1, or 2; and
r is 0 or 1;
with the proviso that R^{c} is not —CN when L is —SCH₂— and R^{d} is optionally substituted phenyl, R^{c} is not C₁-C₆ alkyl when L is —SCH₂— and R^{d} is methyl, and that R^{c} is not —CN when L is —SCH₂— and R^{d} is 2-furyl.

In some embodiments of Formula (Ia), L is —(CH₂)ₘCH₂CH₂—, —(CH₂)ₘY(CH₂)ₚ—, —(CH₂)ₘC(O)(CH₂)ₚ—, —(CH₂)ₘC(O)O(CH₂)ₚ—, —(CH₂)ₘC(O)NR²(CH₂)ₚ—, or —(CH₂)ₘNR²C(O) (CH₂)ₚ. In other embodiments, L is —CH₂CH₂—, —CH₂CH₂CH₂—,—SCH₂—, —SCH₂CH₂—, —CH₂S—, —CH₂SCH₂—, —CH₂CH₂S—, —S(O)CH₂—, —S(O)CH₂CH₂—, —CH₂S(O)—, —CH₂S(O)CH₂—, —CH₂CH₂S(O)—, —S(O)₂CH₂—, —S(O)₂CH₂CH₂—, —CH₂S(O)₂—, —CH₂S(O)₂CH₂—, —CH₂CH₂S(O)₂—, —OCH₂—, —OCH₂CH₂—, —CH₂O—, —CH₂OCH₂—, —CH₂CH₂O—, —NR²CH₂—, CH₂NR²—, —CH₂NR²CH₂—, —CH₂CH₂NR²—, —NR²CH₂CH₂—, —C(O)CH₂—, —C(O)CH₂CH₂—, —C(O)O—, —C(O)OCH₂—, —CH₂C(O)O—, —C(O)NR²—, —C(O)NR²CH₂—, —NR²C(O), —NR²C(O)CH₂, or —CH₂NR₂C(O). In other embodiments, L is —CH₂CH₂—, —CH₂CH₂CH₂—, —SCH₂—, —SCH₂CH₂—, —S(O)CH₂—, —S(O)CH₂CH₂—, —S(O)₂CH₂—, —S(O)₂CH₂CH₂—, —OCH₂—, —OCH₂CH₂—, —NR²CH₂—, —NR²CH₂CH₂—, —C(O)CH₂—, —C(O)CH₂CH₂—, —C(O)O—, —C(O)OCH₂—,—CH₂C(O)O—, —C(O)NR²—, —C(O)NR²CH₂—, —NR²C(O), or —NR²C(O)CH₂. In other embodiments, L is —CH₂CH₂—, —CH₂C(O)—, —C(O)CH₂—, —NR²CH₂—, —CH₂NR²—, —OCH₂—, —CH₂O—, —SCH₂—, —CH₂S—, —S(O)CH₂—, —CH₂S(O)—, —CH₂S(O)₂—, or —S(O)₂CH₂—.

In some embodiments of Formula (Ia), R¹ is C₆-C₁₀ aryl or heteroaryl, wherein the aryl and heteroaryl are substituted with R^{a} and R^{b}, and optionally substituted with one or more R^{e}. In other embodiments, R¹ is C₆-C₁₀ aryl substituted with R^{a} and R^{b}, and optionally substituted with one or more R^{e}. In other embodiments, R¹ is heteroaryl substituted with R^{a} and R^{b}, and optionally substituted with one or more R^{e}. In further embodiments, R¹ is phenyl substituted with R^{a} and R^{b}, and optionally substituted with one or more R^{e}.

In some embodiments of Formula (Ia), R^{a} is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl. In other embodiments, R^{a} is —(CH₂)ᵣCO₂R^{x}, —OCH₂CO₂R^{x}, tetrazole, —(CH₂)tetrazole, oxadiazolone, —(CH₂)oxadiazolone, tetrazolone, —(CH₂)tetrazolone, thiadiazolol, —(CH₂)thiadiazolol, isoxazol-3-ol, —(CH₂) isoxazol-3-ol, —P(O)(OH)OR^{x}, —(CH₂)P(O)(OH)OR^{x}, —S(O)₂OH, —(CH₂)S(O)₂OH, —C(O)NHCN, —(CH₂)C(O)NHCN, —C(O)NHS(O)₂alkyl, or —(CH₂)C(O)NHS(O)₂alkyl. In other embodiments, R^{a} is hydrogen, CO₂R^{x}, CH₂CO₂R^{x}, tetrazole, or oxadiazolone. In further embodiments, R^{a} is hydrogen, CO₂H, CH₂CO₂H, tetrazole, or 1,2,4-oxadiazol-5(4H)-one.

In some embodiments of Formula (Ia), R^{b} is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl. In other embodiments, R^{b} is —(CH₂)ᵣCO₂R^{x}, —OCH₂CO₂R^{x}, tetrazole, —(CH₂)tetrazole, oxadiazolone, —(CH₂)oxadiazolone, tetrazolone, —(CH₂)tetrazolone, thiadiazolol, —(CH₂)thiadiazolol, isoxazol-3-ol, —(CH₂) isoxazol-3-ol, —P(O)(OH)OR^{x}, —(CH₂)P(O)(OH)OR^{x}, —S(O)₂OH, —(CH₂)S(O)₂OH, —C(O)NHCN, —(CH₂)C(O)NHCN, —C(O)NHS(O)₂alkyl, or —(CH₂)C(O)NHS(O)₂alkyl. In other embodiments, R^{b} is hydrogen, CO₂R^{x}, CH₂CO₂R^{x}, tetrazole, or oxadiazolone. In further embodiments, R^{b} is hydrogen, CO₂H, CH₂CO₂H, tetrazole, or 1,2,4-oxadiazol-5(4H)-one. In further embodiments, R^{b} is hydrogen.

In some embodiments of Formula (Ia), R^{c} is H, C₁-C₆ alkyl, C₁-C₆ haloalkyl, halogen, —CN, —OR^{x}, —CO₂R^{x}, or NO₂. In other embodiments, R^{c} is C₁-C₆ alkyl, C₁-C₆ haloalkyl, halogen, —CN, —OR^{x}, —CO₂R^{x}, or NO₂. In other embodiments, R^{c} is halogen, —CN, —OR^{x}, or C₁-C₆ alkyl. In other embodiments, R^{c} is halogen, —CN, —OR^{x}, or C₁-C₃ alkyl. In other embodiments, R^{c} is H, —CN, or halogen. In other embodiments, R^{c} is —CN or halogen.

In some embodiments of Formula (Ia), R^{d} is methyl, optionally substituted 5- to 10-membered aryl, optionally substituted 5- or 6-membered heteroaryl, or optionally substituted 5- or 6-membered carbocycle. In other embodiments, R^{d} is methyl, optionally cyclohexyl, optionally substituted pyridinyl, optionally substituted thiazolyl, optionally substituted phenyl, or optionally substituted thienyl. In other embodiments, R^{d} is cyclohexyl, pyridinyl, thiazolyl, phenyl, or thienyl, wherein each is optionally substituted with one or more substituents independently selected from halogen, C₁-C₆ alkyl, C₁-C₆ hydroxyalkyl, C₁-C₆ alkoxy, C₁-C₆ haloalkyl, C₁-C₆ haloalkoxy, —OH, CN, and amino. In other embodiments, R^{d} is cyclohexyl, pyridinyl, thiazolyl, phenyl, or thienyl, wherein each is optionally substituted with one or more substituents independently selected from halogen, C₁-C₆ alkyl, C₁-C₆ hydroxyalkyl, C₁-C₆ alkoxy, C₁-C₆ haloalkyl, and C₁-C₆ haloalkoxy. In other embodiments, R^{d} is cyclohexyl, pyridinyl, thiazolyl, phenyl, or thienyl, wherein each is optionally substituted with one or more halogen. In other embodiments, R^{d} is methyl, cyclohexyl, pyridinyl, thiazolyl, phenyl, or thienyl. In yet other embodiments, R^{d} is cyclohexyl, pyridinyl, thiazolyl, phenyl, or thienyl. In other embodiments, R^{d} is cyclohexyl, pyridinyl, thiazolyl, phenyl, 4-chlorophenyl, 4-methylphenyl, or thienyl.

In some embodiments of Formula (Ia), each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN. In other embodiments, C₁-C₄ alkyl, C₂-C₄ alkenyl, C₂-C₄ alkynyl, halogen, —OR^{Y}, C₁-C₄ haloalkyl, —NHR^{Z}, —OH, or —CN.

In some embodiments of Formula (Ia), R^{x} is hydrogen or C₁-C₆ alkyl. In other embodiments, R^{x} is hydrogen or C₁-C₃ alkyl. In further embodiments, R^{x} is hydrogen, methyl, ethyl, n-propyl, or iso-propyl.

In some embodiments of Formula (Ia), R^{y} is independently hydrogen, C₁-C₆ alkyl, or C₁-C₆ haloalkyl. In other embodiments, R^{y} is hydrogen, C₁-C₃ alkyl, or C₁-C₃ haloalkyl.

In some embodiments of Formula (Ia), each R^{z} is independently hydrogen, C₁-C₆ alkyl, or C₁-C₆ haloalkyl. In other embodiments, R^{z} is hydrogen, C₁-C₃ alkyl, or C₁-C₃ haloalkyl.

In some embodiments of Formula (Ia), m is 0, 1, or 2. In other embodiments, m is 0. In other embodiments, m is 1. In yet other embodiments, m is 2.

In some embodiments of Formula (Ia), p is 0, 1, or 2. In other embodiments, p is 0. In other embodiments, p is 1. In yet other embodiments, p is 2.

In some embodiments of Formula (Ia), q is 0, 1, or 2. In other embodiments, q is 0. In other embodiments, q is 1. In other embodiments, q is 2.

In some embodiments of Formula (Ia), r is 0 or 1. In other embodiments, r is 0. In other embodiments, r is 1.

In some embodiments of Formula (Ia), one of R^{a} and R^{b} is hydrogen and the other is CO₂R^{x}, CH₂CO₂R^{x}, tetrazole, or oxadiazolone. In other embodiments, R^{b} is hydrogen and R^{a} is CH₂CO₂H, tetrazole, or (1,2,4-oxadiazol-5(4H)-one).

In some embodiments of Formula (Ia), R^{b} is hydrogen, R^{c} is —CN, R^{d} is thienyl, and R^{a} is CH₂CO₂H, tetrazole, or (1,2,4-oxadiazol-5(4H)-one).

In some embodiments of Formula (Ia), R^{c} is halogen, R^{a} is —CO₂H, and R^{b} is H. In other embodiments, R^{c} is —Br, R^{a} is —CO₂H, and R^{b} is H. In further embodiments, R^{c} is —Cl, R^{a} is —CO₂H, and R^{b} is H.

In some embodiments of Formula (Ia), R^{c} is halogen, R^{a} is tetrazole, and R^{b} is H. In other embodiments, R^{c} is —Br, R^{a} is tetrazole, and R^{b} is H. In further embodiments, R^{c} is —Cl, R^{a} is tetrazole, and R^{b} is H.

In some embodiments of Formula (Ia), R^{c} is halogen, R^{a} is —CH₂CO₂H, and R^{b} is H. In other embodiments, R^{c} is —Br, R^{a} is —CH₂CO₂H, and R^{b} is H. In further embodiments, R^{c} is —Cl, R^{a} is —CH₂CO₂H, and R^{b} is H.

In some embodiments of Formula (Ia), R^{c} is halogen, R^{a} is (1,2,4-oxadiazol-5(4H)-one), and R^{b} is H. In other embodiments, R^{c} is —Br, R^{a} is (1,2,4-oxadiazol-5(4H)-one), and R^{b} is H. In other embodiments, R^{c} is —Cl, R^{a} is (1,2,4-oxadiazol-5(4H)-one), and R^{b} is H.

In some embodiments of Formula (Ia), R^{c} is —CN, R^{a} is —CO₂H, and R^{b} is H. In other embodiments, R^{c} is —CN, R^{a} is —CH₂CO₂H, and R^{b} is H. In other embodiments, R^{c} is —CN, R^{a} is tetrazole, and R^{b} is H. In yet other embodiments, R^{c} is —CN, R^{a} is (1,2,4-oxadiazol-5(4H)-one), and R^{b} is H.

In some embodiments of Formula (Ia), R^{c} is not hydrogen or —CN and L is —SCH₂— and R^{d} is optionally substituted phenyl. In other embodiments, R^{c} is not C₁-C₆ alkyl and L is —SCH₂— and R^{d} is methyl. In other embodiments, R^{c} is not —CN and L is —SCH₂— and R^{d} is 2-furyl.

In some embodiments of Formula (Ia), R^{c} is not hydrogen or —CN when L is —SCH₂— and R^{d} is optionally substituted phenyl.

In some embodiments of Formula (Ia), R^{c} is not C₁-C₆ alkyl when L is —SCH₂— and R^{d} is methyl.

In some embodiments of Formula (Ia), R^{c} is not —CN when L is —SCH₂— and R^{d} is 2-furyl.

In another embodiment, the compound of Formula (I) is represented by Formula (Ib):

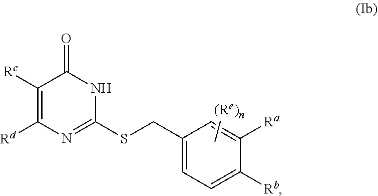

or a pharmaceutically acceptable salt thereof
wherein:
R^{a} and R^{b} is hydrogen and the other is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl;
R^{c} is H, C₁-C₆ alkyl, C₁-C₆ haloalkyl, halogen, —CN, —OR^{x}, —CO₂R^{x}, or NO₂;
R^{d} is methyl, optionally substituted 5- to 10-membered aryl, optionally substituted 5- or 6-membered heteroaryl, or optionally substituted 5- or 6-membered carbocycle;
each R^{x} is independently at each occurrence hydrogen or C₁-C₆ alkyl;
each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN;
each R^{y} and R^{z} is independently hydrogen, C₁-C₆ alkyl, or C₁-C₆ haloalkyl; and
n is 0, 1, 2, or 3;
with the proviso that R^{c} is not hydrogen or —CN when and R^{d} is optionally substituted phenyl, R^{c} is not C₁-C₆ alkyl when R^{d} is methyl, and that R^{c} is not —CN when R^{d} is 2-furyl.

In some embodiments of Formula (Ib),
one of R^{a} and R^{b} is hydrogen and the other is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣ C(O)NHS(O)₂alkyl;
R^{c} is C₁-C₆ alkyl, C₁-C₆ haloalkyl, halogen, —CN, —OR^{x}, —CO₂R^{x}, or NO₂;
R^{d} is methyl, optionally substituted 5- to 10-membered aryl, optionally substituted 5- or 6-membered heteroaryl, or optionally substituted 5- or 6-membered carbocycle;
each R^{x} is independently at each occurrence hydrogen or C₁-C₆ alkyl;
each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN;
with the proviso that R^{c} is not hydrogen or —CN when R^{d} is optionally substituted phenyl, R^{c} is not C₁-C₆ alkyl when R^{d} is methyl, and that R^{c} is not —CN when R^{d} is 2-furyl.

In some embodiments of formula (Ib),
one of R^{a} and R^{b} is hydrogen and the other is CO₂R^{x}, CH₂CO₂R^{x}, tetrazole, or oxadiazolone;
R^{c} is halogen, —CN, —OR^{x}, or C₁-C₆ alkyl;
R^{d} is methyl, optionally substituted 5- to 10-membered aryl, optionally substituted 5- or 6-membered heteroaryl, or optionally substituted 5- or 6-membered carbocycle; and
R^{x} is hydrogen or C₁-C₆ alkyl;
each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN;
each R^{y} and R^{z} is independently hydrogen, C₁-C₆ alkyl, or C₁-C₆ haloalkyl; and
n is 0, 1, 2, or 3;
with the proviso that R^{c} is not —CN when R^{d} is optionally substituted phenyl, R^{c} is not C₁-C₆ alkyl when R^{d} is methyl, and that R^{c} is not —CN when R^{d} is 2-furyl.

In some embodiments of Formula (Ib), R^{a} is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl. In other embodiments, R^{a} is —(CH₂)ᵣCO₂R^{x}, —OCH₂CO₂R^{x}, tetrazole, —(CH₂)tetrazole, oxadiazolone, —(CH₂)oxadiazolone, tetrazolone, —(CH₂)tetrazolone, thiadiazolol, —(CH₂)thiadiazolol, isoxazol-3-ol, —(CH₂)isoxazol-3-ol, —P(O)(OH)OR^{x}, —(CH₂)P(O)(OH)OR^{x}, —S(O)₂OH, —(CH₂)S(O)₂OH, —C(O)NHCN, —(CH₂)C(O)NHCN, —C(O)NHS(O)₂alkyl, or —(CH₂)C(O)NHS(O)₂alkyl. In other embodiments, R^{a} is hydrogen, CO₂R^{x}, CH₂CO₂R^{x}, tetrazole, or oxadiazolone. In further embodiments, R^{a} is hydrogen, CO₂H, CH₂CO₂H, tetrazole, or 1,2,4-oxadiazol-5(4H)-one.

In some embodiments of Formula (Ib), R^{b} is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl. In other embodiments, R^{b} is —(CH₂)ᵣCO₂R^{x}, —OCH₂CO₂R^{x}, tetrazole, —(CH₂)tetrazole, oxadiazolone, —(CH₂)oxadiazolone, tetrazolone, —(CH₂)tetrazolone, thiadiazolol, —(CH₂)thiadiazolol, isoxazol-3-ol, —(CH₂)isoxazol-3-ol, —P(O)(OH)OR^{x}, —(CH₂)P(O)(OH)OR^{x}, —S(O)₂OH, —(CH₂)S(O)₂OH, —C(O)NHCN, —(CH₂)C(O)NHCN, —C(O)NHS(O)₂alkyl, or —(CH₂)C(O)NHS(O)₂alkyl. In other embodiments, R^{b} is hydrogen, CO₂R^{x}, CH₂CO₂R^{x}, tetrazole, or oxadiazolone. In further embodiments, R^{b} is hydrogen, CO₂H, CH₂CO₂H, tetrazole, or 1,2,4-oxadiazol-5(4H)-one. In further embodiments, R^{b} is hydrogen.

In some embodiments of Formula (Ib), R^{c} is H, C₁-C₆ alkyl, C₁-C₆ haloalkyl, halogen, —CN, —OR^{x}, —CO₂R^{x}, or NO₂. In other embodiments, R^{c} is C₁-C₆ alkyl, C₁-C₆ haloalkyl, halogen, —CN, —OR^{x}, —CO₂R^{x}, or NO₂. In other embodiments, R^{c} is halogen, —CN, —OR^{x}, or C₁-C₆ alkyl. In other embodiments, R^{c} is halogen, —CN, —OR^{x}, or C₁-C₃ alkyl. In other embodiments, R^{c} is H, —CN, or halogen. In other embodiments, R^{c} is —CN or halogen.

In some embodiments of Formula (Ib), R^{d} is methyl, optionally substituted 5- to 10-membered aryl, optionally substituted 5- or 6-membered heteroaryl, or optionally substituted 5- or 6-membered carbocycle. In other embodiments, R^{d} is methyl, optionally cyclohexyl, optionally substituted pyridinyl, optionally substituted thiazolyl, optionally substituted phenyl, or optionally substituted thienyl. In other embodiments, R^{d} is cyclohexyl, pyridinyl, thiazolyl, phenyl, or thienyl, wherein each is optionally substituted with one or more substituents independently selected from halogen, C₁-C₆ alkyl, C₁-C₆ hydroxyalkyl, C₁-C₆ alkoxy, C₁-C₆ haloalkyl, C₁-C₆ haloalkoxy, —OH, CN, and amino. In other embodiments, R^{d} is cyclohexyl, pyridinyl, thiazolyl, phenyl, or thienyl, wherein each is optionally substituted with one or more substituents independently selected from halogen, C₁-C₆ alkyl, C₁-C₆ hydroxyalkyl, C₁-C₆ alkoxy, C₁-C₆ haloalkyl, and C₁-C₆ haloalkoxy. In other embodiments, R^{d} is cyclohexyl, pyridinyl, thiazolyl, phenyl, or thienyl, wherein each is optionally substituted with one or more halogen. In other embodiments, R^{d} is methyl, cyclohexyl, pyridinyl, thiazolyl, phenyl, or thienyl. In yet other embodiments, R^{d} is cyclohexyl, pyridinyl, thiazolyl, phenyl, or thienyl. In other embodiments, R^{d} is cyclohexyl, pyridinyl, thiazolyl, phenyl, 4-chlorophenyl, 4-methylphenyl, or thienyl.

In some embodiments of Formula (Ib), each R is independently C₁-C₆ alkyl, C₂-C₆ alkenyl, C₂-C₆ alkynyl, halogen, —OR^{Y}, C₁-C₆ haloalkyl, —NHR^{z}, —OH, or —CN. In other embodiments, C₁-C₄ alkyl, C₂-C₄ alkenyl, C₂-C₄ alkynyl, halogen, —OR^{Y}, C₁-C₄ haloalkyl, —NHR^{z}, —OH, or —CN.

In some embodiments of Formula (Ib), R^{x} is hydrogen or C₁-C₆ alkyl. In other embodiments, R^{x} is hydrogen or C₁-C₃ alkyl. In further embodiments, R^{x} is hydrogen, methyl, ethyl, n-propyl, or iso-propyl.

In some embodiments of Formula (Ib), R^{y} is independently hydrogen, C₁-C₆ alkyl, or C₁-C₆ haloalkyl. In other embodiments, R^{y} is hydrogen, C₁-C₃ alkyl, or C₁-C₃ haloalkyl.

In some embodiments of Formula (Ib), each R^{z} is independently hydrogen, C₁-C₆ alkyl, or C₁-C₆ haloalkyl. In other embodiments, R^{z} is hydrogen, C₁-C₃ alkyl, or C₁-C₃ haloalkyl.

In some embodiments of Formula (Ib), n is 0, 1, 2, or 3. In other embodiments, n is 0 or 1. In further embodiments, n is 0.

In some embodiments of Formula (Ib), one of R^{a} and R^{b} is hydrogen and the other is CO₂R^{x}, CH₂CO₂R^{x}, tetrazole, or oxadiazolone. In other embodiments, R^{b} is hydrogen and R^{a} is CH₂CO₂H, tetrazole, or (1,2,4-oxadiazol-5(4H)-one).

In some embodiments of Formula (Ib), R^{b} is hydrogen, R^{c} is —CN, R^{d} is thienyl, and R^{a} is CH₂CO₂H, tetrazole, or (1,2,4-oxadiazol-5(4H)-one).

In some embodiments of Formula (Ib), R^{c} is halogen, R^{a} is —CO₂H, and R^{b} is H. In other embodiments, R^{c} is —Br, R^{a} is —CO₂H, and R^{b} is H. In further embodiments, R^{c} is —Cl, R^{a} is —CO₂H, and R^{b} is H.

In some embodiments of Formula (Ib), R^{c} is halogen, R^{a} is tetrazole, and R^{b} is H. In other embodiments, R^{c} is —Br, R^{a} is tetrazole, and R^{b} is H. In further embodiments, R^{c} is —Cl, R^{a} is tetrazole, and R^{b} is H.

In some embodiments of Formula (Ib), R^{c} is halogen, R^{a} is —CH₂CO₂H, and R^{b} is H. In other embodiments, R^{c} is —Br, R^{a} is —CH₂CO₂H, and R^{b} is H. In further embodiments, R^{c} is —Cl, R^{a} is —CH₂CO₂H, and R^{b} is H.

In some embodiments of Formula (Ib), R^{c} is halogen, R^{a} is (1,2,4-oxadiazol-5(4H)-one), and R^{b} is H. In other embodiments, R^{c} is —Br, R^{a} is (1,2,4-oxadiazol-5(4H)-one), and R^{b} is H. In other embodiments, R^{c} is —Cl, R^{a} is (1,2,4-oxadiazol-5(4H)-one), and R^{b} is H.

In some embodiments of Formula (Ib), R^{c} is —CN, R^{a} is —CO₂H, and R^{b} is H. In other embodiments, R^{c} is —CN, R^{a} is —CH₂CO₂H, and R^{b} is H. In other embodiments, R^{c} is —CN, R^{a} is tetrazole, and R^{b} is H. In yet other embodiments, R^{c} is —CN, R^{a} is (1,2,4-oxadiazol-5(4H)-one), and R^{b} is H.

In some embodiments of Formula (Ib), R^{c} is not hydrogen or —CN and R^{d} is optionally substituted phenyl. In other embodiments, R^{c} is not C₁-C₆ alkyl and R^{d} is methyl. In other embodiments, R^{c} is not —CN and R^{d} is 2-furyl.

In some embodiments of Formula (Ib), R^{c} is not hydrogen or —CN when and R^{d} is optionally substituted phenyl.

In some embodiments of Formula (Ib), R^{c} is not C₁-C₆ alkyl when R^{d} is methyl.

In some embodiments of Formula (Ib), R^{c} is not —CN when R^{d} is 2-furyl.

In another embodiment, the compound of Formula (I) is represented by Formula (II):

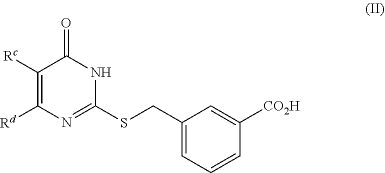

or a pharmaceutically acceptable salt thereof,
wherein:
R^{c} is halogen, —CN, —OR^{x}, or C₁-C₆ alkyl;
R^{d} is methyl, optionally substituted 5- to 10-membered aryl, optionally substituted 5- or 6-membered heteroaryl, or optionally substituted 5- or 6-membered carbocycle; and
R^{x} is hydrogen or C₁-C₆ alkyl
with the proviso that R^{c} is not —CN when and R^{d} is optionally substituted phenyl, R^{c} is not C₁-C₆ alkyl when R^{d} is methyl, and that R^{c} is not —CN when R^{d} is 2-furyl.

In some embodiments of Formula (II), R^{c} is halogen, —CN, —OR^{x}, or C₁-C₆ alkyl; R^{d} is methyl, optionally substituted 5- to 10-membered aryl, optionally substituted 5- or 6-membered heteroaryl, or optionally substituted 5- or 6-membered carbocycle; and R^{x} is hydrogen or C₁-C₆ alkyl with the proviso that R^{c} is not C₁-C₆ alkyl when R^{d} is methyl, and that R^{c} is not —CN when R^{d} is 2-furyl.

In some embodiments of Formula (II), R^{c} is halogen, —CN, —OR^{c}, or C₁-C₆ alkyl. In other embodiments, R^{c} is halogen, —CN, —OR^{x}, or C₁-C₃ alkyl. In further embodiments, R^{c} is —CN or halogen.

In some embodiments of Formula (II), R^{d} is methyl, optionally substituted 5- to 10-membered aryl, optionally substituted 5- or 6-membered heteroaryl, or optionally substituted 5- or 6-membered carbocycle. In other embodiments, R^{d} is cyclohexyl, pyridinyl, thiazolyl, phenyl, or thienyl, wherein each is optionally substituted with one or more substituents independently selected from halogen, C₁-C₆ alkyl, C₁-C₆ hydroxyalkyl, C₁-C₆ alkoxy, C₁-C₆ haloalkyl, C₁-C₆ haloalkoxy, —OH, CN, and amino. In other embodiments, R^{d} is cyclohexyl, pyridinyl, thiazolyl, phenyl, or thienyl, wherein each is optionally substituted with one or more substituents independently selected from halogen, C₁-C₆ alkyl, C₁-C₆ hydroxyalkyl, C₁-C₆ alkoxy, C₁-C₆ haloalkyl, and C₁-C₆ haloalkoxy. In other embodiments, R^{d} is cyclohexyl, pyridinyl, thiazolyl, phenyl, or thienyl, wherein each is optionally substituted with one or more halogen. In further embodiments, R^{d} is methyl, cyclohexyl, pyridinyl, thiazolyl, phenyl, or thienyl.

In some embodiments of Formula (II), R^{b} is hydrogen, CO₂R^{x}, CH₂CO₂R^{x}, tetrazole, or oxadiazolone. In other embodiments, R^{b} is hydrogen, CO₂H, CH₂CO₂H, tetrazole, or 1,2,4-oxadiazol-5(4H)-one. In further embodiments, R^{b} is hydrogen.

In some embodiments of Formula (II), R^{a} is hydrogen, CO₂R^{x}, CH₂CO₂R^{x}, tetrazole, or oxadiazolone. In further embodiments, R^{a} is hydrogen, CO₂H, CH₂CO₂H, tetrazole, or 1,2,4-oxadiazol-5(4H)-one.

In some embodiments of Formula (II), each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN.

In some embodiments of Formula (II), each R^{y} is independently hydrogen, C₁-C₆ alkyl, or C₁-C₆ haloalkyl.

In some embodiments of Formula (II), each R^{z} is independently hydrogen, C₁-C₆ alkyl, or C₁-C₆ haloalkyl.

In some embodiments of Formula (II), n is 0, 1, 2, or 3. In other embodiments, n is 0 or 1. In further embodiments, n is 0.

In some embodiments of Formula (II), one of R^{a} and R^{b} is hydrogen and the other is CO₂R^{x}, CH₂CO₂R^{x}, tetrazole, or oxadiazolone. In other embodiments, R^{b} is hydrogen and R^{a} is CH₂CO₂H, tetrazole, or (1,2,4-oxadiazol-5(4H)-one).

In some embodiments of Formula (II), R^{b} is hydrogen, R^{c} is —CN, R^{d} is thienyl, and R^{a} is CH₂CO₂H, tetrazole, or (1,2,4-oxadiazol-5(4H)-one).

In some embodiments of Formula (II), R^{c} is halogen, R^{a} is —CO₂H, and R^{b} is H. In other embodiments, R^{c} is —Br, R^{a} is —CO₂H, and R^{b} is H. In further embodiments, R^{c} is —Cl, R^{a} is —CO₂H, and R^{b} is H.

In some embodiments of Formula (II), R^{c} is halogen, R^{a} is tetrazole, and R^{b} is H. In other embodiments, R^{c} is —Br, R^{a} is tetrazole, and R^{b} is H. In further embodiments, R^{c} is —Cl, R^{a} is tetrazole, and R^{b} is H.

In some embodiments of Formula (II), R^{c} is halogen, R^{a} is —CH₂CO₂H, and R^{b} is H. In other embodiments, R^{c} is —Br, R^{a} is —CH₂CO₂H, and R^{b} is H. In further embodiments, R^{c} is —Cl, R^{a} is —CH₂CO₂H, and R^{b} is H.

In some embodiments of Formula (II), R^{c} is halogen, R^{a} is (1,2,4-oxadiazol-5(4H)-one), and R^{b} is H. In other embodiments, R^{c} is —Br, R^{a} is (1,2,4-oxadiazol-5(4H)-one), and R^{b} is H. In other embodiments, R^{c} is —Cl, R^{a} is (1,2,4-oxadiazol-5(4H)-one), and R^{b} is H.

In some embodiments of Formula (II), R^{c} is —CN, R^{a} is —CO₂H, and R^{b} is H. In other embodiments, R^{c} is —CN, R^{a} is —CH₂CO₂H, and R^{b} is H. In other embodiments, R^{c} is —CN, R^{a} is tetrazole, and R^{b} is H. In yet other embodiments, R^{c} is —CN, R^{a} is (1,2,4-oxadiazol-5(4H)-one), and R^{b} is H.

In some embodiments of Formula (I), (Ia), (Ib), and (II), one of R^{a} or R^{b} is a carboxylic acid or a carboxylic acid bioisostere.

In some embodiments of Formula (I), (Ia), (Ib), and (II), R^{a} is —CO₂H, —(CH₂)CO₂H, or —OCH₂CO₂H. In other embodiments, R^{a} is —CO₂CH₃, —CO₂CH₂CH₃, —CO₂CH₂CH₂CH₃, —CO₂CH(CH₃)₂, —(CH₂)CO₂CH₃, —(CH₂)CO₂CH₂CH₃, —(CH₂)CO₂CH₂CH₂CH₃, or —(CH₂)CO₂CH(CH₃)₂.

In some embodiments of Formula (I), (Ia), (Ib), and (II), R^{a} is —P(O)(OH)OH, —(CH₂)P(O)(OH)OH, —P(O)(OH)OCH₃, —P(O)(OH)OCH₂CH₃, —P(O)(OH)OCH₂CH₂CH₃, —P(O)(OH)OCH(CH₃)₂, —(CH₂) P(O)(OH)OCH₃, —(CH₂)P(O)(OH)OCH₂CH₃, —(CH₂)P(O)(OH)OCH₂CH₂CH₃, or —(CH₂)P(O)(OH)OCH(CH₃)₂.

In some embodiments of Formula (I), (Ia), (Ib), and (II), R^{a} is —S(O)₂OH, —(CH₂)S(O)₂OH, —C(O)NHCN, or —(CH₂)C(O)NHCN.

In some embodiments of Formula (I), (Ia), (Ib), and (II), R^{a} is —C(O)NHS(O)₂CH₃, —C(O)NHS(O)₂CH₂CH₃, —C(O)NHS(O)₂CH₂CH₂CH₃, —C(O)NHS(O)₂CH(CH₃)₂, —(CH₂)C(O)NHS(O)₂CH₃, —(CH₂)C(O)NHS(O)₂CH₂CH₃, —(CH₂)C(O)NHS(O)₂CH₂CH₂CH₃, or —(CH₂)C(O)NHS(O)₂CH(CH₃)₂.

In some embodiments of Formula (I), (Ia), (Ib), and (II), R^{a} is

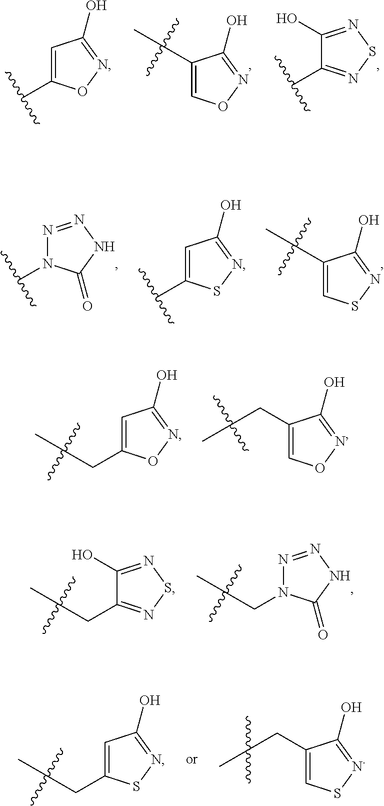

In some embodiments of Formula (I), (Ia). (Ib), and (II), R^{a} is

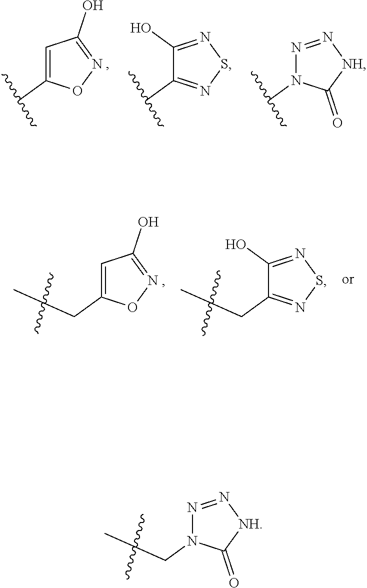

In some embodiments of Formula (I), (Ia), (Ib), and (II), R^{b} is —CO₂H, —(CH₂)CO₂H, or —OCH₂CO₂H. In other embodiments, R^{b} is —CO₂CH₃, —CO₂CH₂CH₃, —CO₂CH₂CH₂CH₃, —CO₂CH(CH₃)₂, —(CH₂)CO₂CH₃, —(CH₂)CO₂CH₂CH₃, —(CH₂)CO₂CH₂CH₂CH₃, or —(CH₂)CO₂CH(CH₃)₂.

In some embodiments of Formula (I), (Ia), (Ib), and (II), R^{b} is —P(O)(OH)OH, —(CH₂)P(O)(OH)OH, —P(O)(OH)OCH₃, —P(O)(OH)OCH₂CH₃, —P(O)(OH)OCH₂CH₂CH₃, —P(O)(OH)OCH(CH₃)₂, —(CH₂) P(O)(OH)OCH₃, —(CH₂)P(O)(OH)OCH₂CH₃, —(CH₂)P(O)(OH)OCH₂CH₂CH₃, or —(CH₂)P(O)(OH)OCH(CH₃)₂.

In some embodiments of Formula (I), (Ia), (Ib), and (II), R^{b} is —S(O)₂OH, —(CH₂)S(O)₂OH, —C(O)NHCN, or —(CH₂)C(O)NHCN.

In some embodiments of Formula (I), (Ia), (Ib), and (II), R^{b} is —C(O)NHS(O)₂CH₃, —C(O)NHS(O)₂CH₂CH₃, —C(O)NHS(O)₂CH₂CH₂CH₃, —C(O)NHS(O)₂CH(CH₃)₂, —(CH₂)C(O)NHS(O)₂CH₃, —(CH₂)C(O)NHS(O)₂CH₂CH₃, —(CH₂)C(O)NHS(O)₂CH₂CH₂CH₃, or —(CH₂)C(O)NHS(O)₂CH(CH₃)₂.

In some embodiments of Formula (I), (Ia), (Ib), and (II), R^{b} is

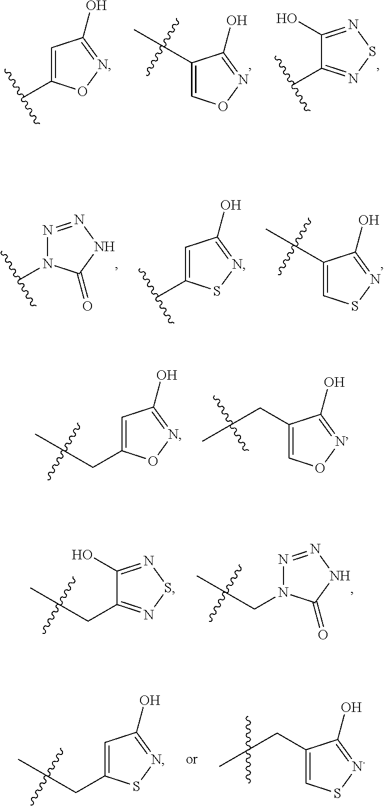

In some embodiments of Formula (I), (Ia), (Ib), and (II), R^{b} is

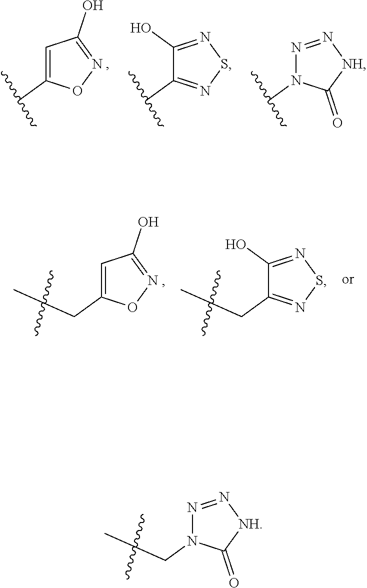

In some embodiments, the present disclosure provides a compound selected from the group consisting of:

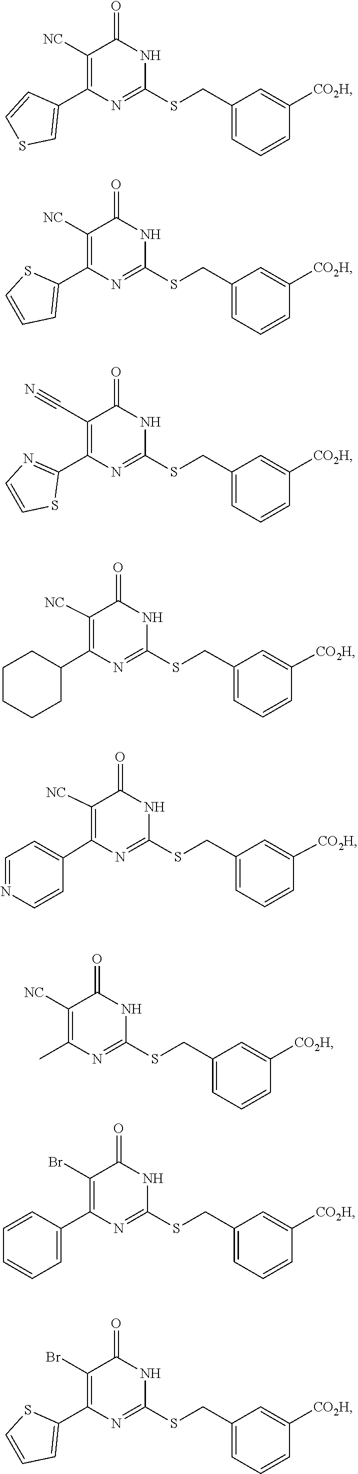

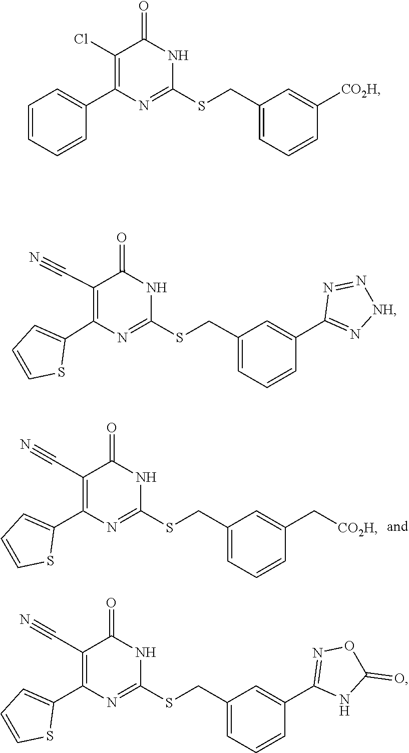 [SMILES: O=C(O)c1cccc(CSc2nc(-c3ccccc3)c(Cl)c(=O)[nH]2)c1 ; N#Cc1c(-c2cccs2)nc(SCc2cccc(-c3nn[nH]n3)c2)[nH]c1=O ; N#Cc1c(-c2cccs2)nc(SCc2cccc(CC(=O)O)c2)[nH]c1=O ; N#Cc1c(-c2cccs2)nc(SCc2cccc(-c3noc(=O)[nH]3)c2)[nH]c1=O]

or a pharmaceutically acceptable salt thereof.

In some embodiments, the present disclosure provides a compound selected from the group consisting of

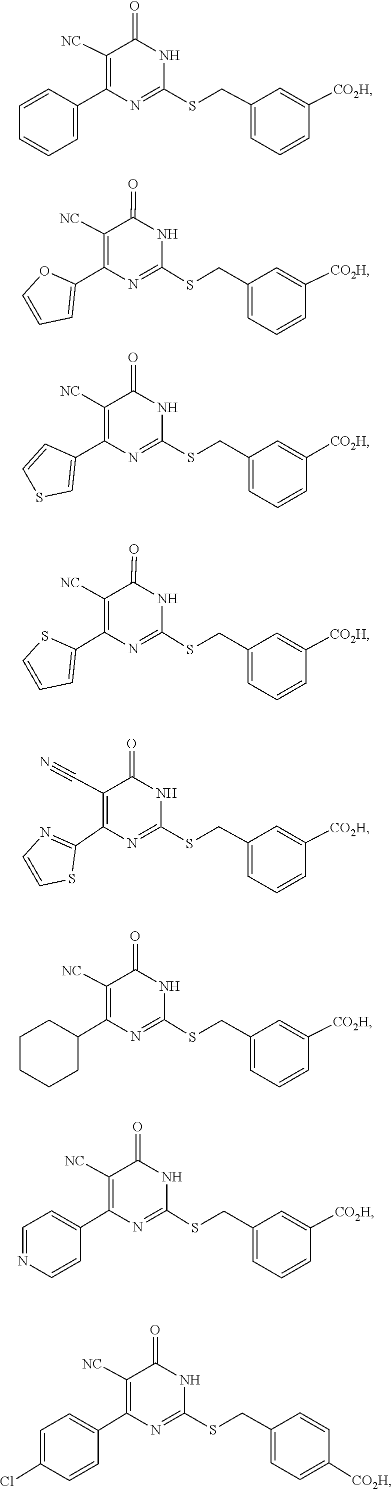

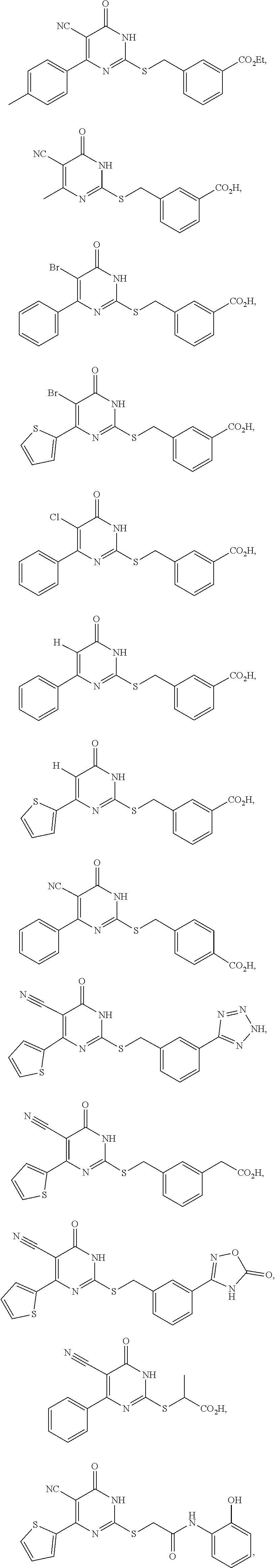

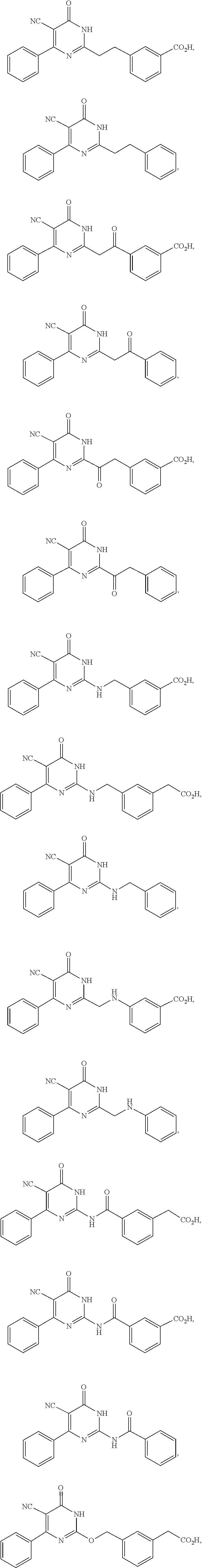

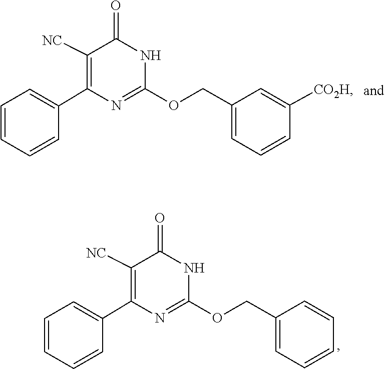 [SMILES: N#Cc1c(-c2ccccc2)nc(OCc2cccc(C(=O)O)c2)[nH]c1=O ; N#Cc1c(-c2ccccc2)nc(OCc2ccccc2)[nH]c1=O]

or a pharmaceutically acceptable salt thereof.

In some embodiments, the present disclosure provides a compound, or a pharmaceutically acceptable salt or tautomer thereof, selected from the group consisting of

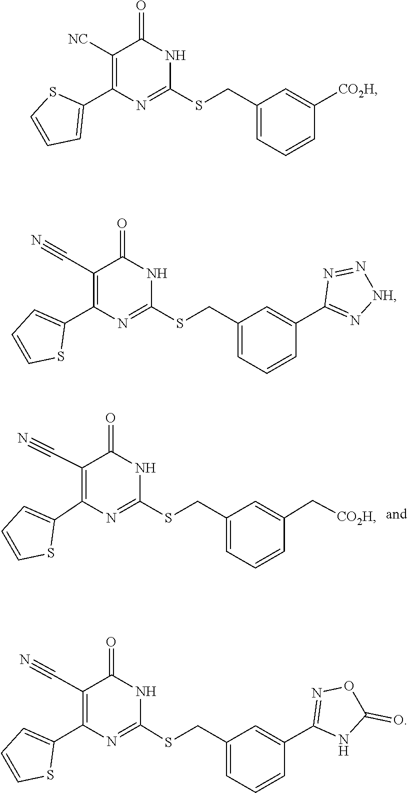

In some embodiments, the present disclosure provides a compound, or a pharmaceutically acceptable salt or tautomer thereof, selected from the group consisting of:

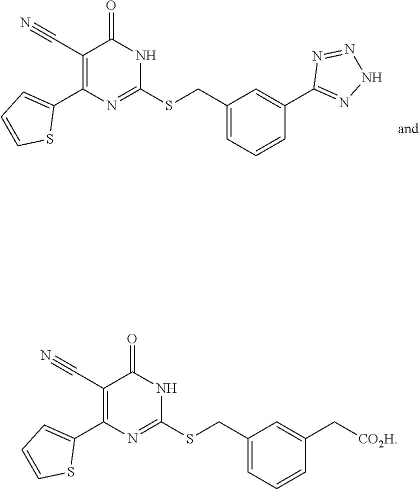

In some embodiments, the present disclosure provides a compound, or a pharmaceutically acceptable salt or tautomer thereof, of the following formula:

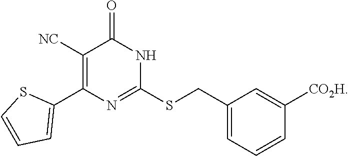 [SMILES: N#Cc1c(-c2cccs2)nc(SCc2cccc(C(=O)O)c2)[nH]c1=O]

In some embodiments, the present disclosure provides a compound, or a pharmaceutically acceptable salt or tautomer thereof, of the following formula:

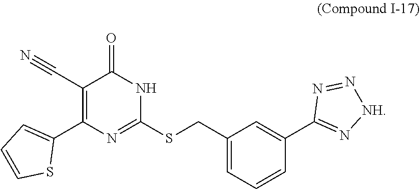

In some embodiments, the present disclosure provides a compound, or a pharmaceutically acceptable salt or tautomer thereof, of the following formula:

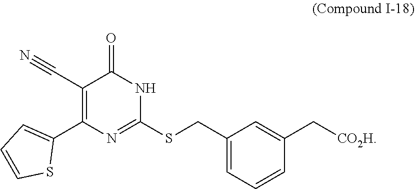 [SMILES: N#Cc1c(-c2cccs2)nc(SCc2cccc(CC(=O)O)c2)[nH]c1=O]

In some embodiments, the present disclosure provides a compound, or a pharmaceutically acceptable salt or tautomer thereof, of the following formula:

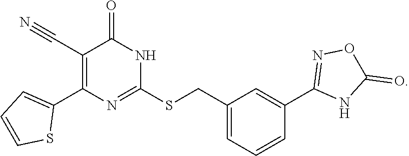 [SMILES: N#Cc1c(-c2cccs2)nc(SCc2cccc(-c3noc(=O)[nH]3)c2)[nH]c1=O]

The above definition of the compounds of Formula (I) is referred to herein by the expressions “compound of Formula (I)” as defined herein, or simply “compounds of Formula (I)”, etc. The above definition of the compounds of Formula (Ia) is referred to herein by the expressions “compound of Formula (Ia)” as defined herein, or simply “compounds of Formula (Ia)”, etc. The above definition of the compounds of Formula (Ib) is referred to herein by the expressions “compound of Formula (Ib)” as defined herein, or simply “compounds of Formula (Ib)”, etc. The above definition of the compounds of Formula (II) is referred to herein by the expressions “compound of Formula (II)” as defined herein, or simply “compounds of Formula (II)”, etc. It should be understood, that such references are intended to encompass not only the above general formula, but also each and every of the embodiments, etc. discussed in the following. It should also be understood, that unless stated to the opposite, such references also encompass isomers, mixtures of isomers, pharmaceutically acceptable salts, solvates and prodrugs of the compounds of Formula (I), Formula (Ia), Formula (Ib), Formula and (II).

The term “alkyl” as used herein refers to a saturated, straight or branched hydrocarbon chain. The hydrocarbon chain preferably contains from one to eight carbon atoms (C₁₋₈-alkyl), more preferred from one to six carbon atoms (C₁₋₆-alkyl), in particular from one to four carbon atoms (C₁₋₄-alkyl), including methyl, ethyl, propyl, isopropyl, butyl, isobutyl, secondary butyl, tertiary butyl, pentyl, isopentyl, neopentyl, tertiary pentyl, hexyl, isohexyl, heptyl, and octyl. In a preferred embodiment “alkyl” represents a C₁₋₄-alkyl group, which may in particular include methyl, ethyl, propyl, isopropyl, butyl, isobutyl, secondary butyl, and tertiary butyl. Correspondingly, the term “alkylene” means the corresponding biradical (-alkyl-).

The term “cycloalkyl” or “carbocycle” as used herein refers to a cyclic alkyl group, preferably containing from three to ten carbon atoms (C₃₋₁₀-cycloalkyl or C₃₋₁₀-carbocycle), such as from three to eight carbon atoms (C₃₋₈-cycloalkyl or C₃₋₁₀-carbocycle), preferably from three to six carbon atoms (C₃₋₆-cycloalkyl or C₃₋₁₀-carbocycle), including cyclopropyl, cyclobutyl, cyclopentyl, cyclohexyl, cycloheptyl, and cyclooctyl. Furthermore, the term “cycloalkyl” as used herein may also include polycyclic groups such as for example bicyclo[2.2.2]octyl, bicyclo[2.2.1]heptanyl, decalinyl, and adamantyl. Correspondingly, the term “cycloalkylene” means the corresponding biradical (-cycloalkyl-). Alkyl and cycloalkyl groups may be optionally substituted with 1-4 substituents. Examples of substituents on alkyl groups include, but are not limited to, alkyl, alkenyl, alkynyl, halogen, haloalkyl, alkoxy, heteroaryl, aryl, carbocyclyl, hydroxyl, carbamoyl, oxo, and —CN.

The term “alkenyl” as used herein refers to a straight or branched hydrocarbon chain or cyclic hydrocarbons containing one or more double bonds, including di-enes, tri-enes and poly-enes. Typically, the alkenyl group comprises from two to eight carbon atoms (C₂₋₈-alkenyl), such as from two to six carbon atoms (C₂₋₆-alkenyl), in particular from two to four carbon atoms (C₂₋₄-alkenyl), including at least one double bond. Examples of alkenyl groups include ethenyl; 1- or 2-propenyl; 1-, 2- or 3-butenyl, or 1,3-but-dienyl; 1-, 2-, 3-, 4- or 5-hexenyl, or 1,3-hex-dienyl, or 1,3,5-hex-trienyl; 1-, 2-, 3-, 4-, 5-, 6-, or 7-octenyl, or 1,3-octadienyl, or 1,3,5-octatrienyl, or 1,3,5,7-octatetraenyl, or cyclohexenyl. Correspondingly, the term “alkenylene” means the corresponding biradical (-alkenyl-). Alkenyl groups may be optionally substituted with 1-4 substituents. Examples of substituents on alkenyl groups include, but are not limited to, alkyl, alkenyl, alkynyl, halogen, haloalkyl, alkoxy, heteroaryl, aryl, carbocyclyl, hydroxyl, carbamoyl, oxo, and —CN.

The term “alkynyl” as used herein refers to a straight or branched hydrocarbon chain containing one or more triple bonds, including di-ynes, tri-ynes and poly-ynes. Typically, the alkynyl group comprises of from two to eight carbon atoms (C₂₋₈-alkynyl), such as from two to six carbon atoms (C₂₋₆-alkynyl), in particular from two to four carbon atoms (C₂₋₄-alkynyl), including at least one triple bond. Examples of preferred alkynyl groups include ethynyl; 1- or 2-propynyl; 1-, 2-, or 3-butynyl, or 1,3-but-diynyl; 1-, 2-, 3-, 4-, or 5-hexynyl, or 1,3-hex-diynyl, or 1,3,5-hex-triynyl; 1-, 2-, 3-, 4-, 5-, 6-, or 7-octynyl, or 1,3-oct-diynyl, or 1,3,5-oct-triynyl, or 1,3,5,7-oct-tetraynyl. Correspondingly, the term “alkynylene” means the corresponding biradical (-alkynyl-). Alkynyl groups may be optionally substituted with 1-4 substituents. Examples of substituents on alkynyl groups include, but are not limited to, alkyl, alkenyl, alkynyl, halogen, haloalkyl, alkoxy, heteroaryl, aryl, carbocyclyl, hydroxyl, carbamoyl, oxo, and —CN.

The terms “halo” and “halogen” as used herein refer to fluoro, chloro, bromo or iodo. Thus a trihalomethyl group represents, e.g., a trifluoromethyl group, or a trichloromethyl group. Preferably, the terms “halo” and “halogen” designate fluoro or chloro.

The term “haloalkyl” as used herein refers to an alkyl group, as defined herein, which is substituted one or more times with one or more halogen. Examples of haloalkyl groups include, but are not limited to, trifluoromethyl, difluoromethyl, pentafluoroethyl, trichloromethyl, etc.

The term “alkoxy” as used herein refers to an “alkyl-O—” group, wherein alkyl is as defined above.

The term “hydroxyalkyl” as used herein refers to an alkyl group (as defined hereinabove), which alkyl group is substituted one or more times with hydroxy. Examples of hydroxyalkyl groups include HO—CH₂—, HO—CH₂—CH₂—, and CH₃—CH(OH)—.

The term “oxy” as used herein refers to an “—O—” group.

The term “oxo” as used herein refers to an “═O” group.

The term “amine” as used herein refers to primary (R—NH₂, R≠H), secondary ((R)₂—NH, (R)₂≠H) and tertiary ((R)₃—N, R≠H) amines. A substituted amine is intended to mean an amine where at least one of the hydrogen atoms has been replaced by the substituent.

The term “carbamoyl” as used herein refers to a “H₂N(C═O)—” group.

The term “aryl”, as used herein, unless otherwise indicated, includes carbocyclic aromatic ring systems derived from an aromatic hydrocarbon by removal of a hydrogen atom. Aryl furthermore includes bi-, tri-, and polycyclic ring systems. Examples of preferred aryl moieties include phenyl, naphthyl, indenyl, indanyl, fluorenyl, biphenyl, indenyl, naphthyl, anthracenyl, phenanthrenyl, pentalenyl, azulenyl, and biphenylenyl. Preferred “aryl” is phenyl, naphthyl, or indanyl, in particular phenyl, unless otherwise stated. Any aryl used may be optionally substituted. Correspondingly, the term “arylene” means the corresponding biradical (-aryl-). Aryl groups may be optionally substituted with 1-4 substituents. Examples of substituents on aryl groups include, but are not limited to, alkyl, alkenyl, alkynyl, halogen, haloalkyl, alkoxy, heteroaryl, aryl, carbocyclyl, hydroxyl, and —CN.

The term “heteroaryl”, as used herein, refers to aromatic groups containing one or more heteroatoms selected from O, S, and N, preferably from one to four heteroatoms, and more preferably from one to three heteroatoms. Heteroaryl furthermore includes bi-, tri- and polycyclic groups, wherein at least one ring of the group is aromatic, and at least one of the rings contains a heteroatom selected from O, S, and N. Heteroaryl also include ring systems substituted with one or more oxo moieties. Examples of preferred heteroaryl moieties include N-hydroxytetrazolyl, N-hydroxytriazolyl, N-hydroxyimidazolyl, furanyl, triazolyl, pyranyl, thiadiazinyl, benzothiophenyl, dihydro-benzo[b]thiophenyl, xanthenyl, isoindanyl, acridinyl, benzisoxazolyl, quinolinyl, isoquinolinyl, phteridinyl, azepinyl, diazepinyl, imidazolyl, thiazolyl, carbazolyl, pyridinyl, pyridazinyl, pyrimidinyl, pyrazolyl, pyrazinyl, tetrazolyl, furyl, thienyl, isoxazolyl, oxazolyl, isothiazolyl, pyrrolyl, indolyl, benzimidazolyl, benzofuranyl, cinnolinyl, indazolyl, indolizinyl, phthalazinyl, triazinyl, isoindolyl, purinyl, oxadiazolyl, thiadiazolyl, furazanyl, benzofurazanyl, benzothiophenyl, benzotriazolyl, benzothiazolyl, benzoxazolyl, quinazolinyl, quinoxalinyl, naphthyridinyl, dihydroquinolyl, tetrahydroquinolyl, dihydroisoquinolyl, tetrahydroisoquinolyl, benzofuryl, furopyridinyl, pyrolopyrimidinyl, azaindolyl, pyrazolinyl, 1,2,4-oxadiazol-5(4H)-one, and pyrazolidinyl. Non-limiting examples of partially hydrogenated derivatives are 1,2,3,4-tetrahydronaphthyl, 1,4-dihydronaphthyl, and I-octalin. Correspondingly, the term “heteroarylene” means the corresponding biradical (-heteroaryl-). Heteroaryl groups may be optionally substituted with 1-4 substituents. Examples of substituents on heteroaryl groups include, but are not limited to, alkyl, alkenyl, alkynyl, halogen, haloalkyl, alkoxy, heteroaryl, aryl, carbocyclyl, hydroxyl, and —CN.

The term “heterocyclyl” as used herein, refers to cyclic non-aromatic groups containing one or more heteroatoms selected from O, S, and N, preferably from one to four heteroatoms, and more preferably from one to three heteroatoms. Heterocyclyl furthermore includes bi-, tri-, and polycyclic non-aromatic groups, and at least one of the rings contains a heteroatom selected from O, S, and N. Heterocyclyl also include ring systems substituted with one or more oxo moieties. Examples of heterocyclic groups are oxetane, pyrrolidinyl, pyrrolyl, 3H-pyrrolyl, oxolanyl, furanyl, thiolanyl, thiophenyl, pyrazolyl, pyrazolidinyl, imidazolyl, imidazolidinyl, 3H-pyrazolyl, 1,2-oxazolyl, 1,3-oxazolyl, 1,2-thiazolyl, 1,3-thiazolyl, 1,2,5-oxadiazolyl, piperidinyl, pyridinyl, oxanyl, 2-H-pyranyl, 4-H-pyranyl, thianyl, 2H-thiopyranyl, pyridazinyl, 1,2-diazinanyl, pyrimidinyl, 1,3-diazinanyl, pyrazinyl, piperazinyl, 1,4-dioxinyl, 1,4-dioxanyl, 1,3-diazinanyl, 1,4-oxazinyl, morpholinyl, thiomorpholinyl, 1,4-oxathianyl, benzofuranyl, isobenzofuranyl, indazolyl, benzimidazolyl, quinolinyl, isoquinolinyl, chromayl, isochromanyl, 4H-chromenyl, 1H-isochromenyl, cinnolinyl, quinazolinyl, quinoxalinyl, phthalazinyl, purinyl, naphthyridinyl, pteridinyl, indolizinyl, 1H-pyrrolizinyl, 4H-quinolizinyl, and aza-8-bicyclo[3.2.1]octane. Correspondingly, the term “heterocyclylene” means the corresponding biradical (-heterocyclyl-). Heterocyclyl groups may be optionally substituted with 1-4 substituents. Examples of substituents on heterocyclyl groups include, but are not limited, to alkyl, alkenyl, alkynyl, halogen, haloalkyl, alkoxy, heteroaryl, aryl, carbocyclyl, hydroxyl, and —CN.

The term “N-heterocyclic ring” as used herein, refers to a heterocyclyl or a heteroaryl, as defined hereinabove, having at least one nitrogen atom, and being bound via a nitrogen atom. Examples of such N-heterocyclic rings are pyrrolidinyl, pyrrolyl, 3H-pyrrolyl, pyrazolyl, pyrazolidinyl, imidazolyl, imidazolidinyl, 3H-pyrazolyl, 1,2-oxazolyl, 1,2-thiazolyl, 1,3-thiazolyl, piperidinyl, pyridinyl, pyridazinyl, pyrazinyl, piperazinyl, morpholinyl, pyridinyl, pyridazinyl, pyrimidinyl, pyrazolyl, pyrazinyl, tetrazolyl, etc.

In the present specification, the structural formula of the compound represents a certain isomer for convenience in some cases, but the present disclosure includes all isomers, such as geometrical isomers, optical isomers based on an asymmetrical carbon, stereoisomers, tautomers, and the like. Accordingly, it should be understood that the definition of compounds of Formulae (I), (Ia), (Ib), and (II) include each and every individual isomer corresponding to the Formula: Formulae (I), (Ia), (Ib), and (II), including cis-trans isomers, stereoisomers and tautomers, as well as racemic mixtures of these and pharmaceutically acceptable salts thereof. Hence, the definition of compounds of Formulae (I), (Ia), (Ib), and (II) are also intended to encompass all R- and S-isomers of a chemical structure in any ratio, e.g., with enrichment (i.e., enantiomeric excess or diastereomeric excess) of one of the possible isomers and corresponding smaller ratios of other isomers. In addition, a crystal polymorphism may be present for the compounds represented by Formulae (I), (Ia), (Ib), and (II). It is noted that any crystal form, crystal form mixture, or anhydride or hydrate thereof is included in the scope of the present disclosure. Furthermore, so-called metabolite which is produced by degradation of the present compound in vivo is included in the scope of the present disclosure.

“Isomerism” means compounds that have identical molecular formulae but differ in the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture”.

A carbon atom bonded to four non-identical substituents is termed a “chiral center”.

Chiral isomer” means a compound with at least one chiral center. Compounds with more than one chiral center may exist either as an individual diastereomer or as a mixture of diastereomers, termed “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J Chem. Soc. 1951 (London), 612; Cahn et al., Experientia 1956, 12, 81; Cahn, J Chem. Educ. 1964, 41, 116).

Diastereoisomers, i.e., non-superimposable stereochemical isomers, can be separated by conventional means such as chromatography, distillation, crystallization or sublimation. The optical isomers can be obtained by resolution of the racemic mixtures according to conventional processes, for example by formation of diastereoisomeric salts by treatment with an optically active acid or base. Examples of appropriate acids include, without limitation, tartaric, diacetyltartaric, dibenzoyltartaric, ditoluoyltartaric, and camphorsulfonic acid. The mixture of diastereomers can be separated by crystallization followed by liberation of the optically active bases from these salts. An alternative process for separation of optical isomers includes the use of a chiral chromatography column optimally chosen to maximize the separation of the enantiomers. Still another available method involves synthesis of covalent diastereoisomeric molecules by reacting compounds of Formula (I), (Ia), (Ib), or (II) with an optically pure acid in an activated form or an optically pure isocyanate. The synthesized diastereoisomers can be separated by conventional means such as chromatography, distillation, crystallization or sublimation, and then hydrolyzed to obtain the enantiomerically pure compound. The optically active compounds of Formulae (I), (Ia), (Ib), and (II) can likewise be obtained by utilizing optically active starting materials and/or by utilizing a chiral catalyst. These isomers may be in the form of a free acid, a free base, an ester, or a salt. Examples of chiral separation techniques are given in Chiral Separation Techniques, A Practical Approach, 2^{nd} ed. by G. Subramanian, Wiley-VCH, 2001.

“Geometric isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Furthermore, the structures and other compounds discussed in this disclosure include all atropic isomers thereof “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques; it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), amine-enamine and enamine-enamine. It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form.

The term “crystal polymorphs”, “polymorphs”, or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical, and electrical properties, stability and solubility. Recrystallization solvent, rate of crystallization, storage temperature, and other factors may cause one crystal form to dominate. Crystal polymorphs of the compounds can be prepared by crystallization under different conditions.

Additionally, the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

The present disclosure is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

All references, including any patent or patent application, cited in this specification are hereby incorporated by reference. No admission is made that any reference constitutes prior art. Further, no admission is made that any of the prior art constitutes part of the common general knowledge in the art.

### Methods for the Preparation of Compounds of Formulae (I), (Ia), (Ib), and (II)

The compounds of the present disclosure (e.g., compounds of Formula (I), Formula (Ia), Formula (Ib), and Formula (II)) can be prepared in a number of ways well known to those skilled in the art of organic synthesis. By way of example, compounds of the present disclosure can be synthesized using the methods described below, together with synthetic methods known in the art of synthetic organic chemistry, or variations thereon as appreciated by those skilled in the art. Preferred methods include but are not limited to those methods described below. The final products of the reactions described herein may be isolated by conventional techniques, e.g., by extraction, crystallisation, distillation, chromatography, etc.

Compounds of the present disclosure can be synthesized by following the steps outlined in General Scheme A to E which comprise different sequences of assembling intermediates Ia-Ih and Ij-Io. Starting materials are either commercially available or made by known procedures in the reported literature or as illustrated. Useful steps that may be used in the preparation steps of the compounds will be known to the skilled person. The method below is given as a non-limiting example on how the compounds may be prepared.

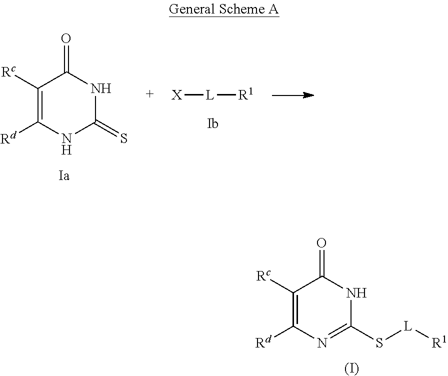

wherein R¹, R^{c}, R^{d}, and L are defined as in Formula (I).

The general way of preparing compounds of Formula (I) by using intermediates Ia, and Ib is outlined in General Scheme A. Coupling of Ta with Tb using a base, i.e., potassium carbonate (K₂CO₃), in a solvent, i.e., acetonitrile (CH₃CN), optionally at elevated temperature provides the desired produce of Formula (I). Bases that can be used include, but are not limited to, sodium carbonate (Na₂CO₃), potassium carbonate (K₂CO₃), N,N-diisopropylethylamine (DIPEA), and triethylamine. Solvents used in the coupling reaction can be polar or non-polar solvents. For example, the solvent can be acetonitrile (CH₃CN), acetone, or dimethylsulfoxide (DMSO).

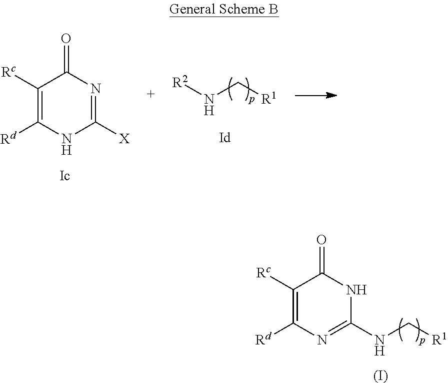

wherein X is a good leaving group, i.e., C₁, Br, —SCH₃, or S(O)₂CH₃, and R¹, R², R^{c}, R^{d}, and p are defined as in Formula (I).

Alternatively, compounds of Formula (I) can be prepared using intermediates Ic and Id as outlined in General Scheme B. Amination of Intermediate Ic with Ie using a base, i.e., sodium hydroxide (NaOH), potassium hydroxide (KOH), etc., in a solvent, i.e., methanol (MeOH), ethanol (EtOH), water (H₂O), etc., provides compounds of Formula (I).

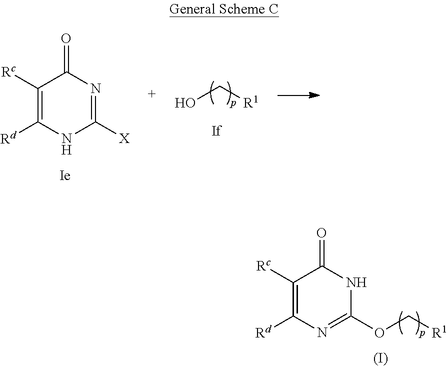

wherein X is a good leaving group, i.e., Cl, Br, —SCH₃, or S(O)₂CH₃, and R¹, R², R^{c}, R^{d}, and p are defined as in Formula (I).

Compounds of Formula (I) can also be prepared using intermediates Ie and If as outlined in General Scheme C. Amination of Intermediate Ie with If using a base, i.e., sodium hydroxide (NaOH), potassium hydroxide (KOH), etc., in a solvent, i.e., methanol (MeOH), ethanol (EtOH), water (H₂O), etc., provides compounds of Formula (I).

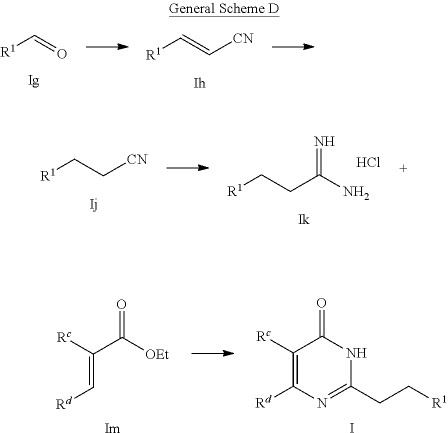

wherein and R¹, R^{c}, and R^{d} are defined as in Formula (I).

Alternatively, compounds of Formula (I) can also be prepared using intermediates Ig, Ih, Ij, Ik, and Im as outlined in General Scheme D. Olefination of intermediate Ig using a base i.e., potassium carbonate (K₂CO₃) and diethyl (cyanomethyl)phosphonate in a solvent, i.e., tetrahydrofuran (THF), water (H₂O), optionally at an elevated temperature provides Intermediate Ih. Hydrogenation of Ih using a metal catalyst, i.e., palladium on carbon (Pd/C), platinum dioxide (PtO₂), etc, and hydrogen (H₂) gas in a solvent, i.e., ethanol (EtOH) and/or tetrahydrofuran (THF), provides Intermediate Ij. Intermediate Ik is obtained by treating Intermediate Ij with an acid, i.e., hydrochloric acid (HCl) in a solvent, i.e., ethanol (EtOH), dichloromethane (CH₂Cl₂), etc., and then subsequent treatment with a base, i.e., ammonia (NH₃). Cyclization of Intermediate Ik and Im using a base, i.e., sodium hydroxide (NaOH), potassium hydroxide (KOH), etc., in a solvent, i.e., dimethylacetamide (DMA), optionally at elevated temperature provides compounds of Formula (I).

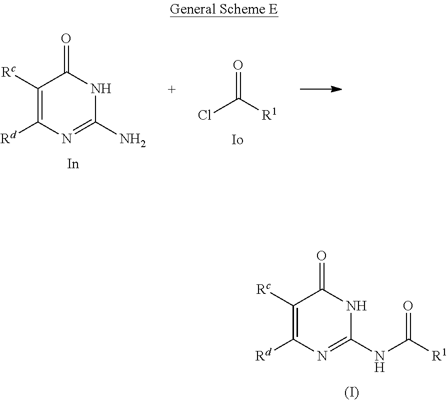

wherein and R¹, R^{c}, and R^{d} are defined as in Formula (I).

Alternatively, compounds of Formula (I) can be prepared using intermediates In and Io as outlined in General Scheme D. Acylation of Intermediate In with Io using a base, i.e., sodium hydroxide (NaOH), potassium hydroxide (KOH), etc., in a solvent, i.e., methanol (MeOH), ethanol (EtOH), water (H₂O), etc., provides compounds of Formula (I).

A mixture of enantiomers, diastereomers, cis/trans isomers resulting from the process described above can be separated into their single components by chiral salt technique, chromatography using normal phase, reverse phase or chiral column, depending on the nature of the separation.

It should be understood that in the description and formula shown above, the various groups R¹, R², X, L, Y, R^{a}, R^{b}, R^{c}, R^{d}, R^{e}, R^{f}, R^{x}, R^{y}, R^{z}, m, n, p, q, r, and other variables are as defined herein above, except where otherwise indicated. Furthermore, for synthetic purposes, the compounds of General Schemes A-E are mere representative with elected radicals to illustrate the general synthetic methodology of the compounds of Formula (I) as defined herein.

### Method of Treatment

The present disclosure provides a method of treating an acute inflammatory condition in a subject comprising administering a compound of Formula (I), Formula (Ia), Formula (Ib), or Formula (II).

Inflammation is a complex of sequential changes expressing the response to damage of cells and vascularized tissues. When tissue injury occurs, whether it is caused by bacteria, trauma, chemicals, heat, or any other phenomenon, the substance histamine, along with other humoral substances, is liberated by the damaged tissue into the surrounding fluids. It is a protective attempt by the organism to remove the injurious stimuli as well as initiating the healing process.

The main features of the inflammatory response are vasodilation, i.e. widening of the blood vessels to increase the blood flow to the infected area; increased vascular permeability which allows diffusible components to enter the site; cellular infiltration by chemotaxis; or the directed movement of inflammatory cells through the walls of blood vessels into the site of injury; changes in biosynthetic, metabolic, and catabolic profiles of many organs; and activation of cells of the immune system as well as of complex enzymatic systems of blood plasma. Inflammation which runs unchecked can, however, lead to a host of diseases, including acute heptatitis, acute pancreatitis, acute kidney disease, inflammatory bowel disease, inflammatory liver diseases, rheumatoid arthritis, autoimmunity, sepsis, SIRS, and atherosclerosis.

Acute inflammation is the initial response of the body to harmful stimuli and is achieved by the increased movement of plasma and leukocytes from the blood into the injured tissues. Acute inflammation can be divided into several phases. The earliest, event of an inflammatory response is temporary vasoconstriction, i.e., narrowing of blood vessels caused by contraction of smooth muscle in the vessel walls which can be seen as blanching (whitening) of the skin. This is followed by several phases that occur minutes, hours, and days later. The first is the acute vascular response which follows within seconds of the tissue injury and lasts for several minutes. This results from vasodilation and increased capillary permeability due to alterations in the vascular endothelium which leads to increased blood flow (hyperemia) that causes redness (erythema) and the entry of fluid into the tissues (edema).

The acute vascular response can be followed by an acute cellular response which takes place over the next few hours. The hallmark of this phase is the appearance of granulocytes, particularly neutrophils, in the tissues. These cells first attach themselves to the endothelial cells within the blood vessels (margination) and then cross into the surrounding tissue (diapedesis). During this phase erythrocytes may also leak into the tissues and a hemorrhage can occur. If the vessel is damaged, fibrinogen and fibronectin are deposited at the site of injury, platelets aggregate and become activated, and the red cells stack together in what are called “rouleau” to help stop bleeding and aid clot formation. The dead and dying cells contribute to pus formation. If the damage is sufficiently severe, a chronic cellular response may follow over the next few days. A characteristic of this phase of inflammation is the appearance of a mononuclear cell infiltrate composed of macrophages and lymphocytes. The macrophages are involved in microbial killing, in clearing up cellular and tissue debris, and in remodeling of tissues.

Acute inflammation occurs immediately upon injury and is a relatively short-term process, lasting up to a few days. Wherein, cytokines and chemokines promote the migration of neutrophils and macrophages to the site of inflammation.

Resident liver macrophages (Kupffer cells) are the first innate immune cells and protect the liver from bacterial infections. Under pathological conditions, they are activated by different components and can differentiate into M1-like (pro-inflammatory) or M2-like (anti-inflammatory) macrophages.

Kupffer cells can be activated to produce a variety of cytokines, eicosanoids, nitric oxide, and oxygen radicals and play divergent roles in tissue injury and tissue repair. Once the acute injury has been controlled, Kupffer cells and other macrophages play a role in suppressing inflammation and initiating wound repair by clearing debr is and producing growth factors and mediators that provide trophic support to the tissue in which they reside.

Kupffer cell activation is involved in the response of the liver to infection or injury; the ensuing inflammatory response protects from infection, as well as limits cellular and organ damage to the host organism.

However, in other types of insults to the liver, the Kupffer cell is unable to appropriately control or resolve its state of activation. The controlled and appropriate resolution of inflammation is an essential feature of the innate immune response. This failure to resolve Kupffer cell activation contributes to a number of chronic inflammatory diseases in the liver.

M1 and M2 macrophage populations differ from their capacity to respond to different stimuli and the repertoire of chemokines/cytokines and receptors they express after their activation. However, both of them become active macrophages with high synthesis and secretion of inflammatory mediators including cytokines, superoxide, nitric oxide, eicosanoids, chemokines, and lysosomal and proteolytic enzymes. Moreover, they exhibit high phagocytic and secretory activities.

Under physiological conditions, Kupffer cells are the first innate immune cells and protect the liver from bacterial infections. Under pathological conditions, they are activated by different components and can differentiate into M1-like (classical) or M2-like (alternative) macrophages. The metabolism of classical or alternative activated Kupffer cells will determine their functions in liver damage.

Kupffer cells are derived from monocytes and differentiate into liver resident macrophages.

Liver-resident Kupffer cells initiate inflammation and help recruit blood-derived monocytes; both differentiate into pro-inflammatory macrophages and further promote NAFLD progression.

While Kupffer Cells display M1-like features in acute liver injury, with protracted chronic inflammation, due to exhaustion of M1-like macrophages and immune cells, M2-like macrophages emerge and secrete protective cytokines upon chronic cytotoxic stimulation such as IL-4, IL-10, and TGF-β.

The immediate resulting effects of liver injuries are increased hepatocellular necrosis, which is one of the principal sources of Kupffer cell activator.

Chronic inflammation is inflammation that lasts for months or years. Macrophages, lymphocytes, and plasma cells predominate in chronic inflammation, in contrast to the neutrophils that predominate in acute inflammation. Examples of diseases mediated by chronic inflammation include diabetes, cardiovascular disease, allergies, and chronic obstructive pulmonary disease (COPD).

Inflammatory cytokines can be divided into two groups: those involved in acute inflammation and those responsible for chronic inflammation. Those involved in acute inflammation include, for example, IL-1, TNF-α, IL-6, IL-11, IL-8, and other chemokines, G-CSF, and GM-CSF. Cytokines in chronic inflammation can be subdivided into cytokines that mediate humoral responses, such as IL-4, IL-5, IL-6, IL-7, and IL-13, and those mediating cellular responses, such as IL-1, IL-2, IL-3, IL-4, IL-7, IL-9, IL-10, IL-12, interferons, transforming growth factor-β, and tumor necrosis factor α and β. Some cytokines contribute to both acute and chronic inflammation.

As used herein, a “cytokine” is a molecule which is released by cells in response to infection or injury that stimulates an inflammatory or healing response. Cytokines are produced by various cells of the body. The cytokine superfamily includes interleukins, chemokines, colony-stimulating factors (CSF), interferons, and the transforming growth factors (TNF) and tumor necrosis factor (TGF) families.

Cytokines are small secreted proteins released by cells have a specific effect on the interactions and communications between cells. Subclasses of cytokines include lymphokine (cytokines made by lymphocytes), monokine (cytokines made by monocytes), chemokine (cytokines with chemotactic activities), and interleukin (cytokines made by one leukocyte and acting on other leukocytes). Cytokines may act on the cells that secrete them (autocrine action), on nearby cells (paracrine action), or in some instances on distant cells (endocrine action). There are both pro-inflammatory cytokines and anti-inflammatory cytokines.

A variety of cytokines are known to induce chemotaxis. One subgroup of cytokines is known as chemokines. These factors represent a family of low molecular weight secreted proteins that primarily function in the activation and migration of leukocytes although some of them also possess a variety of other functions. Chemokines have conserved cysteine residues that allow them to be assigned to four groups: C-C chemokines (RANTES, monocyte chemoattractant protein or MCP-1, monocyte inflammatory protein or MIP-1α, and MIP-10), C-X-C chemokines (IL-8 also called growth related oncogene or GRO/KC), C chemokines (lymphotactin), and CXXXC chemokines (fractalkine).

The net effect of an inflammatory response can be determined by a balance between proinflammatory and anti-inflammatory cytokines. A proinflammatory cytokine is a cytokine which promotes inflammation. Proinflammatory cytokines are produced predominantly by activated macrophages and are involved in the up-regulation of inflammatory reactions. Anti-inflammatory cytokines are a series of immunoregulatory molecules that control the proinflammatory cytokine response. Cytokines act in concert with certain cytokine inhibitors and soluble cytokine receptors to regulate the human immune response.

A proinflammatory cytokine is a cytokine which promotes inflammation. Major proinflammatory cytokines that play roles for early responses are IL-1α, IL-1β, IL-6, and TNF-α. Other proinflammatory mediators include members of the IL-20 family, IL-33 LIF, IFN-γ, OSM, CNTF, TGF-β, GM-CSF, IL-11, IL-12, IL-17, IL-18, IL-8, and a variety of other chemokines that chemoattract inflammatory cells. These cytokines either act as endogenous pyrogens (IL-1, IL-6, TNF-α), upregulate the synthesis of secondary mediators and proinflammatory cytokines by both macrophages and mesenchymal cells (including fibroblasts, epithelial and endothelial cells), stimulate the production of acute phase proteins, or attract inflammatory cells.

Anti-inflammatory cytokines are a series of immunoregulatory molecules that control the proinflammatory cytokine response. Major anti-inflammatory cytokines include interleukin (IL)-1 receptor antagonist, IL-4, IL-10, IL-11, and IL-13. Leukemia inhibitory factor, interferon-α, IL-6, and TGF-β are categorized as either anti-inflammatory or pro-inflammatory cytokines, under various circumstances.

Amongst anti-inflammatory cytokines, IL-10 is a cytokine with anti-inflammatory properties, repressing the expression of inflammatory cytokines, such as TNF-α, IL-6, and IL-1 by activated macrophages. In addition, IL-10 can up-regulate endogenous anti-cytokines and down-regulate pro-inflammatory cytokine receptors.

IL-6 is produced at the site of inflammation and plays a role in the acute phase response as defined by a variety of clinical and biological features such as the production of acute phase proteins. Also, IL-6 in combination with its soluble receptor sIL-6Rα, can dictate the transition from acute to chronic inflammation by changing the nature of leucocyte infiltrate (from polymorphonuclear neutrophils to monocyte/macrophages).

In certain embodiments, the method reduces a pro-inflammatory cytokine or increases an anti-inflammatory cytokine.

In certain embodiments, the pro-inflammatory cytokine is IL-1β, IL-6, IL-18, TNF-α, or TGF-β. In certain embodiments, the pro-inflammatory cytokine is IL-18 or TNF-α. In certain embodiments, the pro-inflammatory cytokine is IL-6. In certain embodiments, the pro-inflammatory cytokine is TGF-β or TNF-α. In certain embodiments, the pro-inflammatory cytokine is IL-1β, IL-6, or TNF-α.

M1 macrophages, also called classically activated, can respond to stimuli, such as LPS or IFN-γ, and are producers of pro-inflammatory cytokines. M2 macrophages, also called alternatively activated, can respond to stimuli such as IL-4 or IL-13, are producer of anti-inflammatory cytokines.

In certain embodiments, the pro-inflammatory cytokine is MCP-1, TNF-α, or IL-1β and the pro-inflammatory cytokine is reduced.

In certain embodiments, the pro-inflammatory cytokine is IL-6 and the pro-inflammatory cytokine is reduced.

In certain embodiments, the anti-inflammatory cytokine is IL-10 (interleukin 10) and the anti-inflammatory cytokine is increased.

SIRT1, a known key metabolic regulator, can reprogram inflammation by altering histones and transcription factors such as NFκB and AP1. Mounting evidence supports that inflammation sequentially links immune, metabolic, and mitochondrial bioenergy networks; sirtuins are essential regulators of these networks.

In certain embodiments, expression of sirtuin-1 modulated genes is increased. In certain embodiments, expression of sirtuin-1 modulated genes is increased in the liver. In certain embodiments, expression of sirtuin-1 modulated genes sod2, tfam, or dda1 are increased. In certain embodiments, expression of sirtuin-1 modulated genes sod2, tfam, or dda1 are increased in the liver.

Inflammation is a cascading event that involves many cellular and humoral mediators. On one hand, suppression of inflammatory responses can leave a subject immunocompromised. However, if left unchecked, inflammation can lead to serious complications including chronic inflammatory diseases (e.g. asthma, psoriasis, arthritis, rheumatoid arthritis, and multiple sclerosis, and the like), septic shock and multiple organ failure. These diverse conditions share common inflammatory mediators, such as cytokines, chemokines, inflammatory cells and other mediators secreted by these cells.

Inflammation may be systemic or may affect a tissue.

In certain embodiments, the acute inflammatory condition is a systemic inflammatory condition. A systemic inflammatory condition refers to a disease or condition with involvement of at least two organ systems.

In one embodiment, systemic inflammatory condition includes SIRS and sepsis. In certain embodiments, the systemic inflammatory condition is one or more of SIRS and sepsis. In certain embodiments, the systemic inflammatory condition is one or more of SIRS, abdominal sepsis, and pulmonary sepsis. In certain embodiments, the systemic inflammatory condition may be associated various infections, including bacterial, viral, or fungal infections. In certain embodiments, the systemic inflammatory condition may be associated with a viral infection, such as COVID.

Systemic inflammatory response syndrome (SIRS) refers to a systemic inflammatory response syndrome with no signs of infection. This condition may also be referred to as “non-infective SIRS” or “infection-free SIRS.” SIRS may be characterised by the presence of at least two of the four following clinical symptoms: fever or hypothermia (temperature of 38.0° C. (100.4° F.) or more, or temperature of 36.0° C. (96.8° F.) or less); tachycardia (at least 90 beats per minute); tachypnea (at least 20 breaths per minute or PaCC>2 less than 4.3 kPa (32.0 mm Hg) or the need for mechanical ventilation); and an altered white blood cell (WBC) count of 12×10⁶ cells/mL or more, or an altered WBC count of 4×10⁶ cells/mL or less, or the presence of more than 10% band forms (immature neutrophils).

Sepsis refers to the systemic inflammatory condition that occurs as a result of infection. Defined focus of infection is indicated by either (i) an organism grown in blood or sterile site; or (ii) an abscess or infected tissue (e.g., pneumonia, peritonitis, urinary tract, vascular line infection, soft tissue). In one embodiment, the infection may be a bacterial infection. The presence of sepsis is also characterised by the presence of at least two (of the four) systemic inflammatory response syndrome (SIRS) criteria defined above.

Cytokine storm or hypercytokinemia is a potentially fatal immune reaction and involves a positive feedback loop between cytokines and immune cells, which causes in the body highly elevated levels of various cytokines. Cytokine storm typically involves increased concentration of cytokines, such as interferons, interleukins, chemokines, colony-stimulating factors, and tumor necrosis factors. Such immune dysregulation can be an underlying factor in mortality resulting from many infections.

Catastrophic antiphospholipid syndrome is a potentially life-threatening condition characterized by diffuse vascular thrombosis, leading to multiple organ failure developing over a short period of time in the presence of positive antiphospholipid antibodies (aPL). It is acute in onset, with majority of cases developing thrombocytopenia, less frequently hemolytic anemia, and disseminated intravascular coagulation. The syndrome is caused by antiphospholipid antibodies that target a group of proteins in the body that are associated with phospholipids. These antibodies activate endothelial cells, platelets, and immune cells, ultimately causing a large inflammatory immune response and widespread clotting.

Graft versus host disease (GVHD) is a syndrome, characterized by inflammation in different organs, with the specificity of epithelial cell apoptosis and crypt drop out. GVHD is commonly associated with bone marrow transplants, stem cell transplants, and other forms of transplanted tissues such as solid organ transplants. White blood cells of a donor's immune system which remain within the donated tissue (the graft) recognize the recipient (the host) as foreign (non-self). The white blood cells present within the transplanted tissue then attack the recipient's body's cells, which leads to GVHD.

In certain embodiments, the acute inflammatory condition is systemic inflammatory response syndrome (SIRS), shock, sepsis, a cytokine storm, or hypercytokinemia, catastrophic anti-phospholipid syndrome, or graft versus host disease (GVHD).

An inflammatory condition includes an inflammatory pulmonary condition. Certain inflammatory pulmonary conditions include infection-induced pulmonary conditions including those associated with viral, bacterial, fungal, parasite, or prion infections. The inflammatory conditions also include community-acquired pneumonia, nosocomial pneumonia, ventilator-associated pneumonia, sepsis, viral pneumonia, influenza infection, parainfluenza infection, rotavirus infection, human metapneumovirus infection, respiratory syncitial virus infection, and aspergillus or other fungal infections. Certain infection-associated inflammatory diseases may include viral or bacterial pneumonia, including severe pneumonia, and acute respiratory distress syndrome (ARDS). Such infection-associated conditions may involve multiple infections such as a primary viral infection and a secondary bacterial infection.

In certain embodiments, the acute inflammatory condition is acute respiratory distress syndrome (ARDS), severe acute respiratory distress syndrome (SARS), viral infections, bacterial infections, fungal infections, influenza, or pneumonia.

In certain embodiments, the acute inflammatory condition is a cytokine storm or hypercytokinemia, systemic inflammatory response syndrome (SIRS), graft versus host disease (GVHD), acute respiratory distress syndrome (ARDS), severe acute respiratory distress syndrome (SARS), catastrophic anti-phospholipid syndrome, viral infections, bacterial infections, fungal infections, influenza, pneumonia, shock, or sepsis.

In certain embodiments, the acute inflammatory condition is an organ-specific or tissue-specific condition. Certain affected tissues are pancreas, hepatic tissue, the respiratory tract, lung, the gastrointestinal tract, small intestine, large intestine, colon, rectum, the cardiovascular system, cardiac tissue, blood vessels, joint, bone and synovial tissue, cartilage, epithelium, endothelium, or adipose tissue.

In certain embodiments, the acute inflammatory condition is acute pancreatitis, hepatitis, respiratory condition, or enterocolitis.

### Pharmaceutical Compositions

The compound of Formula (I), Formula (Ia), Formula (Ib), or Formula (II) may be provided in any form suitable for the intended administration, in particular including pharmaceutically acceptable salts, solvates and prodrugs of the compound of Formula (I), Formula (Ia), Formula (Ib), or Formula (II).

Pharmaceutically acceptable salts refer to salts of the compounds of Formula (I), Formula (Ia), Formula (Ib), or Formula (II) which are considered to be acceptable for clinical and/or veterinary use. Typical pharmaceutically acceptable salts include those salts prepared by reaction of the compounds of Formula (I), Formula (Ia), Formula (Ib), or Formula (II) and a mineral or organic acid or an organic or inorganic base. Such salts are known as acid addition salts and base addition salts, respectively. It will be recognized that the particular counter-ion forming a part of any salt is not of a critical nature, so long as the salt as a whole is pharmaceutically acceptable and as long as the counter-ion does not contribute undesired qualities to the salt as a whole. These salts may be prepared by methods known to the skilled person. Pharmaceutically acceptable salts are, e.g., those described and discussed in Remington's Pharmaceutical Sciences, 17. Ed. Alfonso R. Gennaro (Ed.), Mack Publishing Company, Easton, PA, U.S.A., 1985 and more recent editions and in Encyclopedia of Pharmaceutical Technology.

Examples of pharmaceutically acceptable addition salts include acid addition salts formed with inorganic acids, e.g., hydrochloric, hydrobromic, sulfuric, nitric, hydroiodic, metaphosphoric, or phosphoric acid; and organic acids e.g., succinic, maleic, acetic, fumaric, citric, tartaric, benzoic, trifluoroacetic, malic, lactic, formic, propionic, glycolic, gluconic, camphorsulfuric, isothionic, mucic, gentisic, isonicotinic, saccharic, glucuronic, furoic, glutamic, ascorbic, anthranilic, salicylic, phenylacetic, mandelic, embonic (pamoic), ethanesulfonic, pantothenic, stearic, sulfinilic, alginic and galacturonic acid; and arylsulfonic, for example benzenesulfonic, p-toluenesulfonic, methanesulfonic or naphthalenesulfonic acid; and base addition salts formed with alkali metals and alkaline earth metals and organic bases such as N,N-dibenzylethylenediamine, chloroprocaine, choline, diethanolamine, ethylenediamine, meglumine (N-methylglucamine), lysine and procaine; and internally formed salts. It should be understood that all references to pharmaceutically acceptable salts include solvent addition forms (solvates) or crystal forms (polymorphs) as defined herein, of the same salt.

The compound of Formula (I), Formula (Ia), Formula (Ib), or Formula (II), or a pharmaceutically acceptable salt thereof, may be provided in dissoluble or indissoluble forms together with a pharmaceutically acceptable solvent such as water, ethanol, and the like. Dissoluble forms may also include hydrated forms such as the mono-hydrate, the dihydrate, the hemihydrate, the trihydrate, the tetrahydrate, and the like.

The compound of Formula (I), Formula (Ia), Formula (Ib), or Formula (II), or a pharmaceutically acceptable salt thereof, may be provided as a prodrug. The term “prodrug” used herein is intended to mean a compound which—upon exposure to certain physiological conditions—will liberate the compound of Formula (I), Formula (Ia), Formula (Ib), or Formula (II), or a pharmaceutically acceptable salt thereof, which then will be able to exhibit the desired biological action. A typical example is a labile carbamate of an amine.

Since prodrugs are known to enhance numerous desirable qualities of pharmaceuticals (e.g., solubility, bioavailability, manufacturing, etc.), the compounds of the present disclosure can be delivered in prodrug form. Thus, the present disclosure is intended to cover prodrugs of the presently claimed compounds, methods of delivering the same and compositions containing the same. “Prodrugs” are intended to include any covalently bonded carriers that release an active parent drug of the present disclosure in vivo when such prodrug is administered to a subject. Prodrugs in the present disclosure are prepared by modifying functional groups present in the compound in such a way that the modifications are cleaved, either in routine manipulation or in vivo, to the parent compound. Prodrugs include compounds of the present disclosure wherein a hydroxy, amino, sulfhydryl, carboxy or carbonyl group is bonded to any group that may be cleaved in vivo to form a free hydroxyl, free amino, free sulfhydryl, free carboxy, or free carbonyl group, respectively.

Examples of prodrugs include, but are not limited to, esters (e.g., acetate, dialkylaminoacetates, formates, phosphates, sulfates, and benzoate derivatives) and carbamates (e.g., N,N-dimethylaminocarbonyl) of hydroxy functional groups, esters (e.g., C₁₋₆ alkyl esters, e.g., methyl esters, ethyl esters, 2-propyl esters, phenyl esters, 2-aminoethyl esters, morpholinoethanol esters, etc.) of carboxyl functional groups, N-acyl derivatives (e.g., N-acetyl)N-Mannich bases, Schiff bases and enaminones of amino functional groups, oximes, acetals, ketals and enol esters of ketone and aldehyde functional groups in compounds of the disclosure, and the like. See Bundegaard, H., Design of Prodrugs, p 1-92, Elesevier, New York-Oxford (1985).

The compounds, or pharmaceutically acceptable salts, esters or prodrugs thereof, are administered orally, nasally, transdermally, pulmonary, inhalationally, buccally, sublingually, intraperintoneally, subcutaneously, intramuscularly, intravenously, rectally, intrapleurally, intrathecally, and parenterally. In one embodiment, the compound is administered orally. One skilled in the art will recognize the advantages of certain routes of administration.

The dosage regimen utilizing the compounds is selected in accordance with a variety of factors including type, species, age, weight, sex, and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

In one aspect of this disclosure, there is provided a pharmaceutical composition comprising at, as an active ingredient, at least one compound of Formula (I), Formula (Ia), Formula (Ib), or Formula (II), or a pharmaceutically acceptable salt thereof, as defined herein, and optionally one or more pharmaceutically acceptable excipients, diluents and/or carriers. The compounds of Formula (I), Formula (Ia), Formula (Ib), or Formula (II), or a pharmaceutically acceptable salt thereof, may be administered alone or in combination with pharmaceutically acceptable carriers, diluents, or excipients, in either single or multiple doses. Suitable pharmaceutically acceptable carriers, diluents and excipients include inert solid diluents or fillers, sterile aqueous solutions and various organic solvents.

A “pharmaceutical composition” is a formulation containing the compounds of the present disclosure in a form suitable for administration to a subject. The pharmaceutical compositions may be formulated with pharmaceutically acceptable carriers or diluents as well as any other known adjuvants and excipients in accordance with conventional techniques such as those disclosed in Remington: The Science and Practice of Pharmacy, 21st Edition, 2000, Lippincott Williams & Wilkins.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

The pharmaceutical compositions formed by combining a compound of Formula (I), Formula (Ia), Formula (Ib), or Formula (II), or a pharmaceutically acceptable salt thereof, as defined herein, with pharmaceutically acceptable carriers, diluents or excipients can be readily administered in a variety of dosage forms such as tablets, powders, lozenges, syrups, suppositories, injectable solutions and the like. In powders, the carrier is a finely divided solid such as talc or starch which is in a mixture with the finely divided active component. In tablets, the active component is mixed with the carrier having the necessary binding properties in suitable proportions and compacted in the shape and size desired.

The pharmaceutical compositions may be specifically prepared for administration by any suitable route such as the oral and parenteral (including subcutaneous, intramuscular, intrathecal, intravenous and intradermal) route. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders, and granules. Where appropriate, they can be prepared with coatings such as enteric coatings or they can be prepared so as to provide controlled release of the active ingredient such as sustained or prolonged release according to methods well known in the art.

For oral administration in the form of a tablet or capsule, a compound of Formula (I), Formula (Ia), Formula (Ib), or Formula (II), or a pharmaceutically acceptable salt thereof, as defined herein, may suitably be combined with an oral, non-toxic, pharmaceutically acceptable carrier such as ethanol, glycerol, water, or the like. Furthermore, suitable binders, lubricants, disintegrating agents, flavouring agents, and colourants may be added to the mixture, as appropriate. Suitable binders include, e.g., lactose, glucose, starch, gelatin, acacia gum, tragacanth gum, sodium alginate, carboxymethylcellulose, polyethylene glycol, waxes, or the like. Lubricants include, e.g., sodium oleate, sodium stearate, magnesium stearate, sodium benzoate, sodium acetate, sodium chloride, or the like. Disintegrating agents include, e.g., starch, methyl cellulose, agar, bentonite, xanthan gum, sodium starch glycolate, crospovidone, croscarmellose sodium, or the like. Additional excipients for capsules include macrogels or lipids.

For the preparation of solid compositions such as tablets, the active compound of Formula (I), Formula (Ia), Formula (Ib), or Formula (II), or a pharmaceutically acceptable salt thereof, is mixed with one or more excipients, such as the ones described above, and other pharmaceutical diluents such as water to make a solid pre-formulation composition containing a homogenous mixture of a compound of Formula (I), Formula (Ia), Formula (Ib), or Formula (II), or a pharmaceutically acceptable salt thereof. The term “homogenous” is understood to mean that the compound of Formula (I), Formula (Ia), Formula (Ib), or Formula (II), or a pharmaceutically acceptable salt thereof, is dispersed evenly throughout the composition so that the composition may readily be subdivided into equally effective unit dosage forms such as tablets or capsules.

Liquid compositions for either oral or parenteral administration of the compound of Formula (I), Formula (Ia), Formula (Ib), or Formula (II), or a pharmaceutically acceptable salt thereof, include, e.g., aqueous solutions, syrups, elixirs, aqueous or oil suspensions and emulsion with edible oils such as cottonseed oil, sesame oil, coconut oil, or peanut oil. Suitable dispersing or suspending agents for aqueous suspensions include synthetic or natural gums such as tragacanth, alginate, acacia, dextran, sodium carboxymethylcellulose, gelatin, methylcellulose, or polyvinylpyrrolidone.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use.

For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

The preparation of all these solutions under sterile conditions is readily accomplished by standard pharmaceutical techniques well known to those skilled in the art.

For example, sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. Depot injectable compositions are also contemplated as being within the scope of the present disclosure.

For parenteral administration, solutions containing a compound of Formula (I), Formula (Ia), Formula (Ib), or Formula (II), or a pharmaceutically acceptable salt thereof, in sesame or peanut oil, aqueous propylene glycol, or in sterile aqueous solution may be employed. Such aqueous solutions should be suitably buffered if necessary and the liquid diluent first rendered isotonic with sufficient saline or glucose. These particular aqueous solutions are especially suitable for intravenous, intramuscular, subcutaneous and intraperitoneal administration. The oily solutions are suitable for intra-articular, intra-muscular and subcutaneous injection purposes.

In addition to the aforementioned ingredients, the compositions of a compound of Formula (I), Formula (Ia), Formula (Ib), or Formula (II), or a pharmaceutically acceptable salt thereof, may include one or more additional ingredients such as diluents, buffers, flavouring agents, colourant, surface active agents, thickeners, preservatives, e.g., methyl hydroxybenzoate (including anti-oxidants), emulsifying agents and the like.

The term “therapeutically effective amount”, as used herein, refers to an amount of a pharmaceutical agent to treat, ameliorate, or prevent an identified disease, disorder, or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or disorder to be treated is a disease or disorder associated with α-amino-β-carboxymuconate-ε-semialdehyde decarboxylase (ACMSD) dysfunction. In a preferred aspect, the disease or disorder is treated by inhibition of α-amino-β-carboxymuconate-ε-semialdehyde decarboxylase (ACMSD). In certain embodiments, the disease or disorder is an acute inflammatory condition.

For any compound, the therapeutically effective amount can be estimated initially either in cell culture assays, e.g., in cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

A suitable dosage of the compound of Formula (I), Formula (Ia), Formula (Ib), or Formula (II), or a pharmaceutically acceptable salt thereof, will depend on the age and condition of the patient, the severity of the disease to be treated and other factors well known to the practicing physician. The compound may be administered for example either orally, parenterally or topically according to different dosing schedules, e.g., daily or with intervals, such as weekly intervals. In general a single dose will be in the range from 0.01 to 500 mg/kg body weight, preferably from about 0.05 to 100 mg/kg body weight, more preferably between 0.1 to 50 mg/kg body weight, and most preferably between 0.1 to 25 mg/kg body weight. The compound may be administered as a bolus (i.e., the entire daily dose is administered at once) or in divided doses two or more times a day. Variations based on the aforementioned dosage ranges may be made by a physician of ordinary skill taking into account known considerations such as weight, age, and condition of the person being treated, the severity of the affliction, and the particular route of administration.

As used herein, a “subject” or “subject in need thereof” is a subject having a disease or disorder that is an acute inflammatory condition. In other embodiments, a subject has a disease or disorder associated with α-amino-β-carboxymuconate-F-semialdehyde decarboxylase (ACMSD) modulation. In other embodiments, a subject has a disease or disorder associated with modulation of NAD⁺ levels. A “subject” includes a mammal. The mammal can be e.g., any mammal, e.g., a human, primate, bird, mouse, rat, fowl, dog, cat, cow, horse, goat, camel, sheep, or a pig. Preferably, the mammal is a human.

The compounds of Formula (I), Formula (Ia), Formula (Ib), or Formula (II), or a pharmaceutically acceptable salt thereof, may also be prepared in a pharmaceutical composition comprising one or more further active substances alone, or in combination with pharmaceutically acceptable carriers, diluents, or excipients in either single or multiple doses. The suitable pharmaceutically acceptable carriers, diluents and excipients are as described herein above, and the one or more further active substances may be any active substances, or preferably an active substance as described in the section “combination treatment” herein below.

### EXEMPLARY EMBODIMENTS

Embodiment I-1. A method of treating an acute inflammatory condition in a subject comprising administering to the subject a therapeutically effective amount of a compound represented by Formula (I):

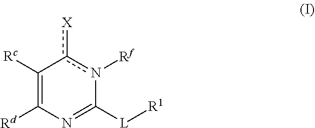

or a pharmaceutically acceptable salt or tautomer thereof,
wherein:
X is O or OH;
L is —(CH₂)ₘCH₂CH₂—, —(CH₂)ₘY(CH₂)ₚ—, —(CH₂)ₘC(O)(CH₂)ₚ—, —(CH₂)ₘC(O)O(CH₂)ₚ—, —(CH₂)ₘC(O)NR²(CH₂)ₚ—, or —(CH₂)ₘNR²C(O)(CH₂)ₚ;
Y is O, N or S(O)q;
R¹ is C₆-C₁₀ aryl or heteroaryl, wherein the aryl and heteroaryl are substituted with R^{a} and R^{b}, and optionally substituted with one or more R^{e};
R² is H or C₁-C₆ alkyl;
one of R^{a} and R^{b} is hydrogen and the other is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl;
R^{c} is H, C₁-C₆ alkyl, C₁-C₆ haloalkyl, halogen, —CN, —OR^{x}, —CO₂R^{x}, or NO₂;
R^{d} is methyl, optionally substituted 5- to 10-membered aryl, optionally substituted 5- or 6-membered heteroaryl, or optionally substituted 5- or 6-membered carbocycle;
each R^{x} is independently at each occurrence hydrogen or C₁-C₆ alkyl;
each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN;
R^{f} is H or absent;
each R^{y} and R^{z} is independently hydrogen, C₁-C₆ alkyl, or C₁-C₆ haloalkyl;
each m and p independently is 0, 1, or 2, wherein m+p<3;
q is 0, 1, or 2;
r is 0 or 1; and
the dotted line is an optional double bond;
with the proviso that R^{c} is not hydrogen or —CN when X is O, L is —SCH₂— and R^{d} is optionally substituted phenyl, R^{c} is not C₁-C₆ alkyl when X is O, L is —SCH₂— and R^{d} is methyl, and that R^{c} is not —CN when X is O, L is —SCH₂— and R^{d} is 2-furyl.

Embodiment I-1a. A method of treating an acute inflammatory condition in a subject comprising administering to the subject a therapeutically effective amount of a compound represented by Formula (I):

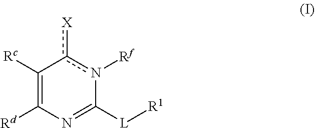

or a pharmaceutically acceptable salt or tautomer thereof,
wherein:
X is O or OH;
L is —(CH₂)ₘCH₂CH₂—, —(CH₂)ₘY(CH₂)ₚ—, —(CH₂)ₘC(O)(CH₂)ₚ—, —(CH₂)ₘC(O)O(CH₂)ₚ—, —(CH₂)ₘC(O)NR²(CH₂)ₚ—, or —(CH₂)ₘNR²C(O)(CH₂)ₚ;
Y is O, N or S(O)q;
R¹ is C₆-C₁₀ aryl or heteroaryl, wherein the aryl and heteroaryl are substituted with R^{a} and R^{b}, and optionally substituted with one or more R^{e};
R² is H or C₁-C₆ alkyl;
one of R^{a} and R^{b} is hydrogen and the other is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣdihydrotetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl;
R^{c} is H, C₁-C₆ alkyl, C₁-C₆ haloalkyl, halogen, —CN, —OR^{x}, —CO₂R^{x}, or NO₂;
R^{d} is methyl, optionally substituted 5- to 10-membered aryl, optionally substituted 5- or 6-membered heteroaryl, or optionally substituted 5- or 6-membered carbocycle;
each R^{x} is independently at each occurrence hydrogen or C₁-C₆ alkyl;
each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN;
R^{f} is H or absent;
each R^{y} and R^{z} is independently hydrogen, C₁-C₆ alkyl, or C₁-C₆ haloalkyl;
each m and p independently is 0, 1, or 2, wherein m+p<3;
q is 0, 1, or 2;
r is 0 or 1; and
the dotted line is an optional double bond;
with the proviso that R^{c} is not hydrogen or —CN when X is O, L is —SCH₂— and R^{d} is optionally substituted phenyl, R^{c} is not C₁-C₆ alkyl when X is O, L is —SCH₂— and R^{d} is methyl, and that R^{c} is not —CN when X is O, L is —SCH₂— and R^{d} is 2-furyl.

Embodiment I-2. The method of Embodiment I-1, wherein the compound is represented by Formula (Ia)

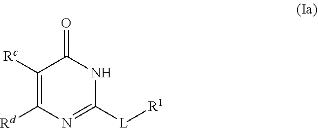

or a pharmaceutically acceptable salt, or tautomer thereof.

Embodiment I-3. The method of Embodiment I-1 or I-2, wherein the compound is represented by Formula (Ib):

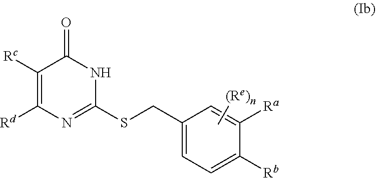

or a pharmaceutically acceptable salt thereof, wherein n is 0, 1, 2, or 3.

Embodiment I-4. The method of any one of Embodiments I-1 to I-3,
wherein:
one of R^{a} and R^{b} is hydrogen and the other is CO₂R^{x}, CH₂CO₂R^{x}, tetrazole, or oxadiazolone;
R^{c} is halogen, —CN, —OR^{x}, or C₁-C₆ alkyl;
R^{d} is methyl, optionally substituted 5- to 10-membered aryl, optionally substituted 5- or 6-membered heteroaryl, or optionally substituted 5- or 6-membered carbocycle; and
R^{x} is hydrogen or C₁-C₆ alkyl;
each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN;
each R^{y} and R^{z} is independently hydrogen, C₁-C₆ alkyl, or C₁-C₆ haloalkyl; and
n is 0, 1, 2, or 3;
with the proviso that R^{c} is not hydrogen or —CN when R^{d} is optionally substituted phenyl and that R^{c} is not —CN when R^{d} is 2-furyl.

Embodiment I-5. The method of any one of Embodiments I-1 to I-3, wherein the compound is represented by Formula (II):

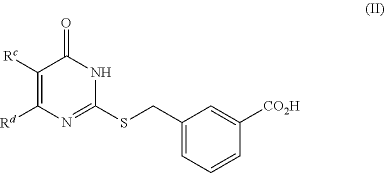

or a pharmaceutically acceptable salt thereof.

Embodiment I-6. The method of Embodiment I-5, wherein R^{c} is halogen, —CN, —OR^{x}, or C₁-C₆ alkyl, R^{d} is methyl, optionally substituted 5- to 10-membered aryl, optionally substituted 5- or 6-membered heteroaryl, or optionally substituted 5- or 6-membered carbocycle, and R^{x} is hydrogen or C₁-C₆ alkyl.

Embodiment I-7. The method of any one of Embodiments I-1 to I-6, wherein R^{c} is —CN or halogen.

Embodiment I-8. The method of any one of Embodiments I-1 to I-7, wherein R^{d} is methyl, cyclohexyl, pyridinyl, thiazolyl, phenyl, or thienyl.

Embodiment I-9. The method of any one of Embodiments I-1 to I-7, wherein R^{d} is methyl, cyclohexyl, pyridinyl, thiazolyl, thienyl, or optionally substituted phenyl.

Embodiment I-10. The method of any one of Embodiments I-1 to I-4, wherein R^{a} is hydrogen, CH₂CO₂H, tetrazole, or oxadiazolone (1,2,4-oxadiazol-5(4H)-one).

Embodiment I-11. The method of any one of Embodiments I-1 to I-4, wherein R^{b} is hydrogen, CH₂CO₂H, tetrazole, or oxadiazolone (1,2,4-oxadiazol-5(4H)-one).

Embodiment I-12. The method of any one of Embodiments I-1 to I-4, wherein n is 0.

Embodiment I-13. The method of Embodiment I-1, wherein the compound is selected from the group consisting of:

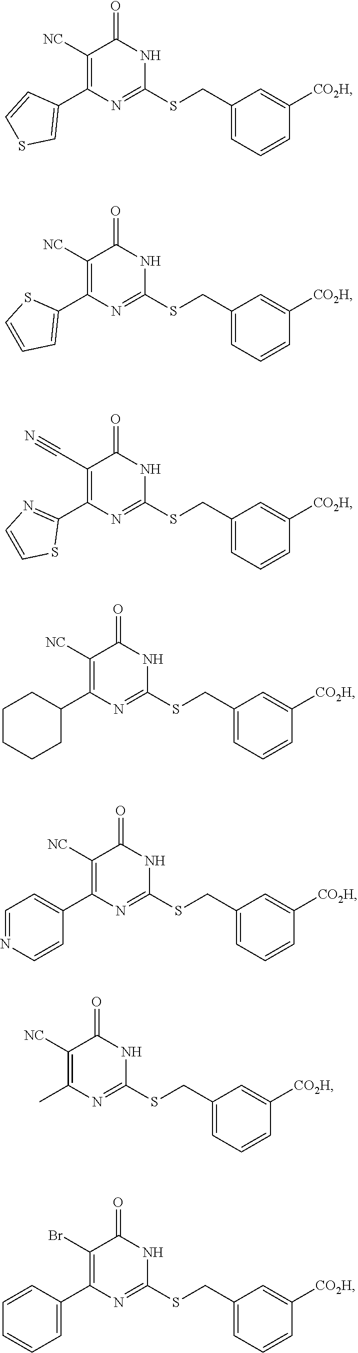

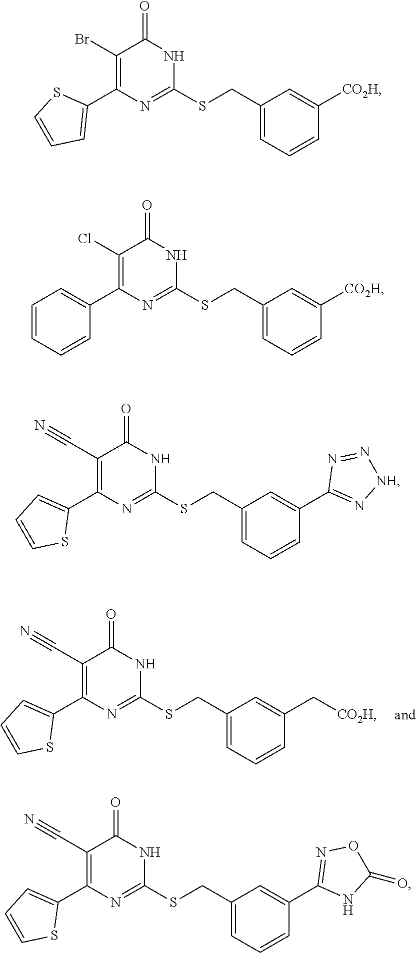 [SMILES: O=C(O)c1cccc(CSc2nc(-c3cccs3)c(Br)c(=O)[nH]2)c1 ; O=C(O)c1cccc(CSc2nc(-c3ccccc3)c(Cl)c(=O)[nH]2)c1 ; N#Cc1c(-c2cccs2)nc(SCc2cccc(-c3nn[nH]n3)c2)[nH]c1=O ; N#Cc1c(-c2cccs2)nc(SCc2cccc(CC(=O)O)c2)[nH]c1=O ; N#Cc1c(-c2cccs2)nc(SCc2cccc(-c3noc(=O)[nH]3)c2)[nH]c1=O]

or a pharmaceutically acceptable salt thereof.

Embodiment I-14. The method of Embodiment I-1, wherein the compound is selected from the group consisting of

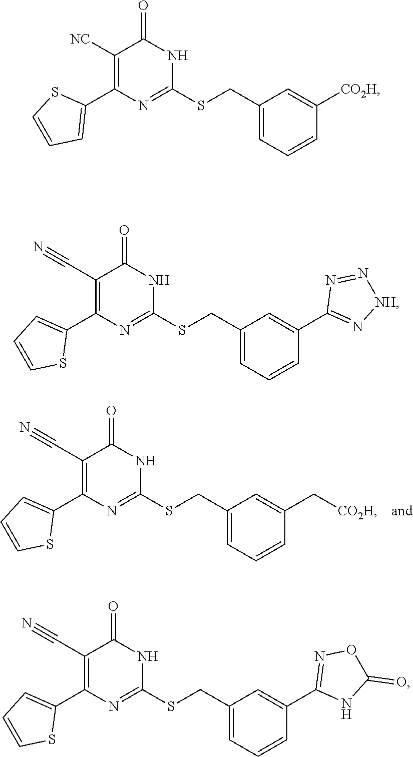 [SMILES: N#Cc1c(-c2cccs2)nc(SCc2cccc(C(=O)O)c2)[nH]c1=O ; N#Cc1c(-c2cccs2)nc(SCc2cccc(-c3nn[nH]n3)c2)[nH]c1=O ; N#Cc1c(-c2cccs2)nc(SCc2cccc(CC(=O)O)c2)[nH]c1=O ; N#Cc1c(-c2cccs2)nc(SCc2cccc(-c3noc(=O)[nH]3)c2)[nH]c1=O]

or a pharmaceutically acceptable salt thereof.

Embodiment I-15. The method of Embodiment I-1, wherein the compound is selected from the group consisting of

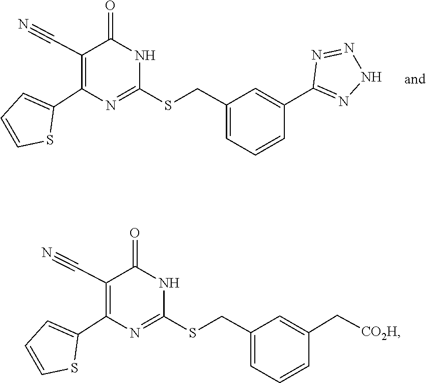 [SMILES: N#Cc1c(-c2cccs2)nc(SCc2cccc(-c3nn[nH]n3)c2)[nH]c1=O ; N#Cc1c(-c2cccs2)nc(SCc2cccc(CC(=O)O)c2)[nH]c1=O]

or a pharmaceutically acceptable salt thereof.

Embodiment I-16. The method of Embodiment I-1, wherein the compound is

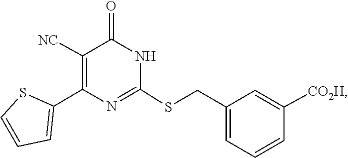 [SMILES: N#Cc1c(-c2cccs2)nc(SCc2cccc(C(=O)O)c2)[nH]c1=O]

or a pharmaceutically acceptable salt thereof.

Embodiment I-17. The method of Embodiment I-1, wherein the compound is

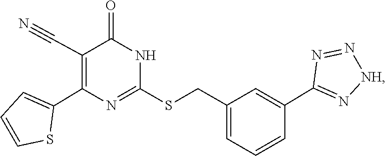 [SMILES: N#Cc1c(-c2cccs2)nc(SCc2cccc(-c3nn[nH]n3)c2)[nH]c1=O]

or a pharmaceutically acceptable salt thereof.

Embodiment I-18. The method of Embodiment I-1, wherein the compound is

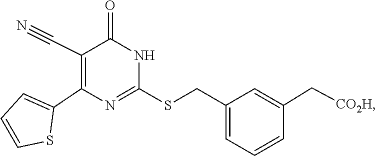 [SMILES: N#Cc1c(-c2cccs2)nc(SCc2cccc(CC(=O)O)c2)[nH]c1=O]

or a pharmaceutically acceptable salt thereof.

Embodiment I-19. The method of Embodiment I-1, wherein the compound is

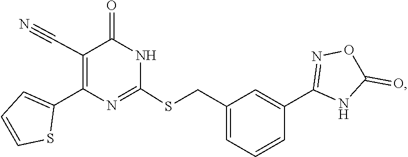 [SMILES: N#Cc1c(-c2cccs2)nc(SCc2cccc(-c3noc(=O)[nH]3)c2)[nH]c1=O]

or a pharmaceutically acceptable salt thereof.

Embodiment I-20. The method of any one of Embodiments I-1 to I-19, wherein the acute inflammatory condition is a systemic inflammatory condition.

Embodiment I-21. The method of any one of Embodiments I-1 to I-19, wherein the acute inflammatory condition is an organ-specific condition.

Embodiment I-22. The method of any one of Embodiments I-1 to I-19, wherein the acute inflammatory condition is a cytokine storm or hypercytokinemia, systemic inflammatory response syndrome (SIRS), graft versus host disease (GVHD), acute respiratory distress syndrome (ARDS), severe acute respiratory distress syndrome (SARS), catastrophic anti-phospholipid syndrome, viral infections, bacterial infections, fungal infections, influenza, pneumonia, shock, or sepsis.

Embodiment I-23. The method of any one of Embodiments I-1 to I-19, wherein the acute inflammatory condition is acute pancreatitis, hepatitis, respiratory condition, or enterocolitis.

Embodiment I-24. The method of any one of Embodiments I-1 to I-23, wherein the method reduces a pro-inflammatory cytokine or increases an anti-inflammatory cytokine.

Embodiment I-25. The method of Embodiment I-24, wherein the pro-inflammatory cytokine is IL-1β, IL-6, IL-18, TNF-α, or TGF-β.

Embodiment I-26. The method of Embodiment I-24, wherein pro-inflammatory cytokine is MCP-1, TNF-α, or IL-1β.

Embodiment I-27. The method of Embodiment I-24, wherein pro-inflammatory cytokine is IL-6.

Embodiment I-28. The method of Embodiment I-24, wherein the anti-inflammatory cytokine is IL-10.

Embodiment I-29. The method of any one of Embodiment I-1 to I-23, wherein expression of sirtuin-1 modulated genes sod2, tfam, dda1 genes are increased in the liver.

Embodiment I-30. A compound represented by Formula (I):

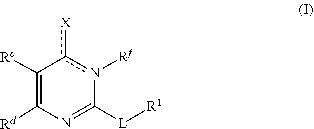

or a pharmaceutically acceptable salt or tautomer thereof,
wherein:
X is O or OH;
L is —(CH₂)ₘCH₂CH₂—, —(CH₂)ₘY(CH₂)ₚ—, —(CH₂)ₘC(O)(CH₂)ₚ—, —(CH₂)ₘC(O)O(CH₂)ₚ—, —(CH₂)ₘC(O)NR²(CH₂)ₚ—, or —(CH₂)ₘNR²C(O)(CH₂)ₚ;
Y is O, N or S(O)q;
R¹ is C₆-C₁₀ aryl or heteroaryl, wherein the aryl and heteroaryl are substituted with R^{a} and R^{b}, and optionally substituted with one or more R^{e};
R² is H or C₁-C₆ alkyl;
one of R^{a} and R is hydrogen and the other is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣdihydrotetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl;
R^{c} is H, C₁-C₆ alkyl, C₁-C₆ haloalkyl, halogen, —CN, —OR^{x}, —CO₂R^{x}, or NO₂;
R^{d} is methyl, optionally substituted 5- to 10-membered aryl, optionally substituted 5- or 6-membered heteroaryl, or optionally substituted 5- or 6-membered carbocycle;
each R^{x} is independently at each occurrence hydrogen or C₁-C₆ alkyl;
each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN;
R^{f} is H or absent;
each R^{y} and R^{z} is independently hydrogen, C₁-C₆ alkyl, or C₁-C₆ haloalkyl;
each m and p independently is 0, 1, or 2, wherein m+p<3;
q is 0, 1, or 2;
r is 0 or 1; and
the dotted line is an optional double bond;
with the proviso that R^{c} is not hydrogen or —CN when X is O, L is —SCH₂— and R^{d} is optionally substituted phenyl, R^{c} is not C₁-C₆ alkyl when X is O, L is —SCH₂— and R^{d} is methyl, and that R^{c} is not —CN when X is O, L is —SCH₂— and R^{d} is 2-furyl; for use in treating an acute inflammatory condition.

Embodiment I-31. The compound for use of Embodiment I-30, wherein the compound is represented by Formula (Ia)

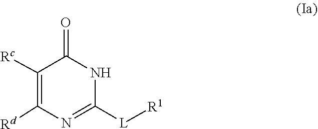

or a pharmaceutically acceptable salt, or tautomer thereof.

Embodiment I-32. The compound for use of Embodiment I-30 or I-31, wherein the compound is represented by Formula (Ib):

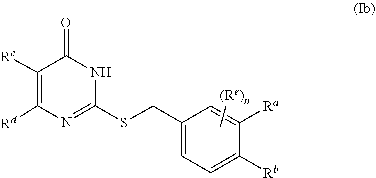

or a pharmaceutically acceptable salt thereof, wherein n is 0, 1, 2, or 3.

Embodiment I-33. The compound for use of any one of Embodiments I-30 to I-32,
wherein:
one of R^{a} and R^{b} is hydrogen and the other is CO₂R^{x}, CH₂CO₂R^{x}, tetrazole, or oxadiazolone;
R^{c} is halogen, —CN, —OR^{x}, or C₁-C₆ alkyl;
R^{d} is methyl, optionally substituted 5- to 10-membered aryl, optionally substituted 5- or 6-membered heteroaryl, or optionally substituted 5- or 6-membered carbocycle; and
R^{x} is hydrogen or C₁-C₆ alkyl;
each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN;
each R^{y} and R^{z} is independently hydrogen, C₁-C₆ alkyl, or C₁-C₆ haloalkyl; and
n is 0, 1, 2, or 3;
with the proviso that R^{c} is not hydrogen or —CN when R^{d} is optionally substituted phenyl and that R^{c} is not —CN when R^{d} is 2-furyl.

Embodiment I-34. The compound for use of any one of Embodiments I-30 to I-32, wherein the compound is represented by Formula (II):

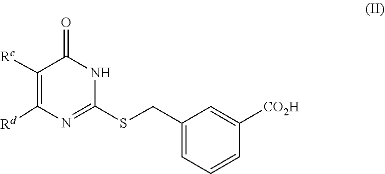

or a pharmaceutically acceptable salt thereof.

Embodiment I-35. The compound for use of Embodiment I-34, wherein R^{c} is halogen, —CN, —OR^{x}, or C₁-C₆ alkyl, R^{d} is methyl, optionally substituted 5- to 10-membered aryl, optionally substituted 5- or 6-membered heteroaryl, or optionally substituted 5- or 6-membered carbocycle, and R^{x} is hydrogen or C₁-C₆ alkyl.

Embodiment I-36. The compound for use of any one of Embodiments I-30 to I-35, wherein R^{c} is —CN or halogen.

Embodiment I-37. The compound for use of any one of Embodiments I-30 to I-36, wherein R^{d} is methyl, cyclohexyl, pyridinyl, thiazolyl, phenyl, or thienyl.

Embodiment I-38. The compound for use of any one of Embodiments I-30 to I-36, wherein R^{d} is methyl, cyclohexyl, pyridinyl, thiazolyl, thienyl, or optionally substituted phenyl.

Embodiment I-39. The compound for use of any one of Embodiments I-30 to I-33, wherein R^{a} is hydrogen, CH₂CO₂H, tetrazole, or oxadiazolone (1,2,4-oxadiazol-5(4H)-one).

Embodiment I-40. The compound for use of any one of Embodiments I-30 to I-33, wherein R^{b} is hydrogen, CH₂CO₂H, tetrazole, or oxadiazolone (1,2,4-oxadiazol-5(4H)-one).

Embodiment I-41. The compound for use of any one of Embodiments I-30 to I-33, wherein n is 0.

Embodiment I-42. The compound for use of Embodiment I-30, wherein the compound is selected from the group consisting of

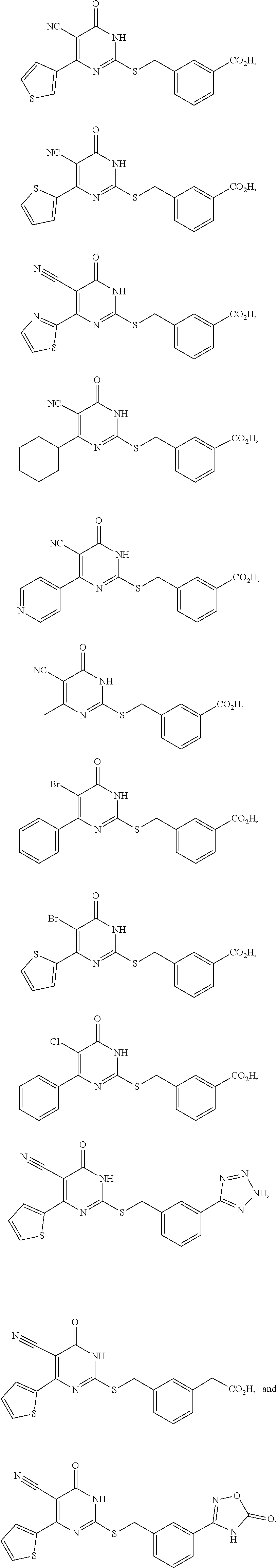

or a pharmaceutically acceptable salt thereof.

Embodiment I-43. The compound for use of Embodiment I-30, wherein the compound is selected from the group consisting of

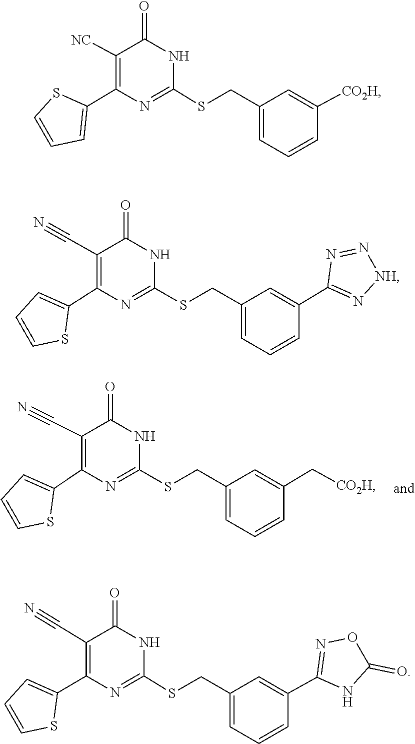 [SMILES: N#Cc1c(-c2cccs2)nc(SCc2cccc(C(=O)O)c2)[nH]c1=O ; N#Cc1c(-c2cccs2)nc(SCc2cccc(-c3nn[nH]n3)c2)[nH]c1=O ; N#Cc1c(-c2cccs2)nc(SCc2cccc(CC(=O)O)c2)[nH]c1=O ; N#Cc1c(-c2cccs2)nc(SCc2cccc(-c3noc(=O)[nH]3)c2)[nH]c1=O]

or a pharmaceutically acceptable salt thereof.

Embodiment I-44. The compound for use of Embodiment I-30, wherein the compound is selected from the group consisting of

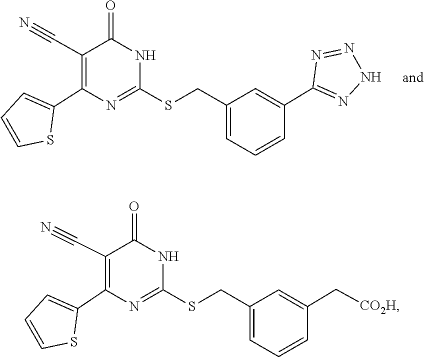 [SMILES: N#Cc1c(-c2cccs2)nc(SCc2cccc(-c3nn[nH]n3)c2)[nH]c1=O ; N#Cc1c(-c2cccs2)nc(SCc2cccc(CC(=O)O)c2)[nH]c1=O]

or a pharmaceutically acceptable salt thereof.

Embodiment I-45. The compound for use of Embodiment I-30, wherein the compound is

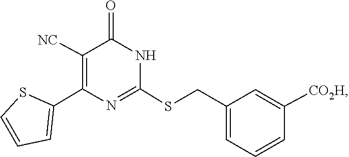 [SMILES: N#Cc1c(-c2cccs2)nc(SCc2cccc(C(=O)O)c2)[nH]c1=O]

or a pharmaceutically acceptable salt thereof.

Embodiment I-46. The compound for use of Embodiment I-30, wherein the compound is

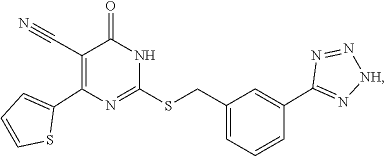 [SMILES: N#Cc1c(-c2cccs2)nc(SCc2cccc(-c3nn[nH]n3)c2)[nH]c1=O]

or a pharmaceutically acceptable salt thereof.

Embodiment I-47. The compound for use of Embodiment I-30, wherein the compound is

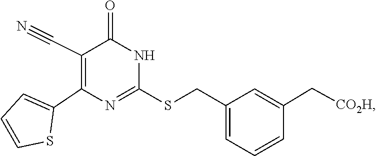 [SMILES: N#Cc1c(-c2cccs2)nc(SCc2cccc(CC(=O)O)c2)[nH]c1=O]

or a pharmaceutically acceptable salt thereof.

Embodiment I-48. The compound for use of Embodiment I-30, wherein the compound is

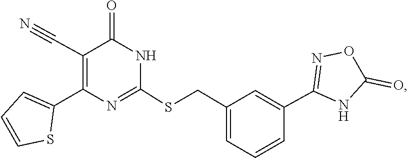 [SMILES: N#Cc1c(-c2cccs2)nc(SCc2cccc(-c3noc(=O)[nH]3)c2)[nH]c1=O]

or a pharmaceutically acceptable salt thereof.

Embodiment I-49. The compound for use of any one of Embodiments I-30 to I-48, wherein the acute inflammatory condition is a systemic inflammatory condition.

Embodiment I-50. The compound for use of any one of Embodiments I-30 to I-48, wherein the acute inflammatory condition is an organ-specific condition.

Embodiment I-51. The compound for use of any one of Embodiments I-30 to I-48, wherein the acute inflammatory condition is a cytokine storm or hypercytokinemia, systemic inflammatory response syndrome (SIRS), graft versus host disease (GVHD), acute respiratory distress syndrome (ARDS), severe acute respiratory distress syndrome (SARS), catastrophic anti-phospholipid syndrome, viral infections, bacterial infections, fungal infections, influenza, pneumonia, shock, or sepsis.

Embodiment I-52. The compound for use of any one of Embodiments I-30 to I-48, wherein the acute inflammatory condition is acute pancreatitis, hepatitis, respiratory condition, or enterocolitis.

Embodiment I-53. The compound for use of any one of Embodiments I-30 to I-52, wherein a pro-inflammatory cytokine is reduced or an anti-inflammatory cytokine is increased.

Embodiment I-54. The compound for use of Embodiment I-53, wherein the pro-inflammatory cytokine is IL-1β, IL-6, IL-18, TNF-α, or TGF-β.

Embodiment I-55. The compound for use of Embodiment I-53, wherein pro-inflammatory cytokine is MCP-1, TNF-α, or IL-1β.

Embodiment I-56. The compound for use of Embodiment I-53, wherein pro-inflammatory cytokine is IL-6.

Embodiment I-57. The compound for use of Embodiment I-53, wherein the anti-inflammatory cytokine is IL-10.

Embodiment I-58. The compound for use of any one of Embodiments I-30 to I-52, wherein expression of sirtuin-1 modulated genes sod2, tfam, dda1 genes are increased in the liver.

Embodiment I-59. A use of a compound represented by Formula (I):

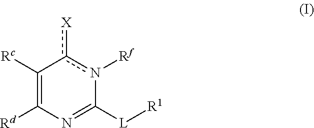

or a pharmaceutically acceptable salt or tautomer thereof,
wherein:
X is O or OH;
L is —(CH₂)ₘCH₂CH₂—, —(CH₂)ₘY(CH₂)ₚ—, —(CH₂)ₘC(O)(CH₂)ₚ—, —(CH₂)ₘC(O)O(CH₂)ₚ—, —(CH₂)ₘC(O)NR²(CH₂)ₚ—, or —(CH₂)ₘNR²C(O)(CH₂)ₚ;
Y is O, N or S(O)_{q};
R¹ is C₆-C₁₀ aryl or heteroaryl, wherein the aryl and heteroaryl are substituted with R^{a} and R^{b}, and optionally substituted with one or more R^{e};
R² is H or C₁-C₆ alkyl;
one of R^{a} and R^{b} is hydrogen and the other is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣdihydrotetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl;
R^{c} is H, C₁-C₆ alkyl, C₁-C₆ haloalkyl, halogen, —CN, —OR^{x}, —CO₂R^{x}, or NO₂;
R^{d} is methyl, optionally substituted 5- to 10-membered aryl, optionally substituted 5- or 6-membered heteroaryl, or optionally substituted 5- or 6-membered carbocycle;
each R^{x} is independently at each occurrence hydrogen or C₁-C₆ alkyl;
each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN;
R^{f} is H or absent;
each R^{y} and R^{z} is independently hydrogen, C₁-C₆ alkyl, or C₁-C₆ haloalkyl;
each m and p independently is 0, 1, or 2, wherein m+p<3;
q is 0, 1, or 2;
r is 0 or 1; and
the dotted line is an optional double bond;
with the proviso that R^{c} is not hydrogen or —CN when X is O, L is —SCH₂— and R^{d} is optionally substituted phenyl, R^{c} is not C₁-C₆ alkyl when X is O, L is —SCH₂— and R^{d} is methyl, and that R^{c} is not —CN when X is O, L is —SCH₂— and R^{d} is 2-furyl;
for treating an acute inflammatory condition.

Embodiment I-60. The use of Embodiment I-59, wherein the compound is represented by Formula (Ia)

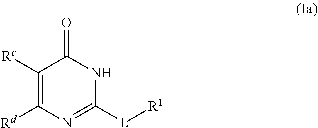

or a pharmaceutically acceptable salt, or tautomer thereof.

Embodiment I-61. The use of Embodiment I-59 or I-60, wherein the compound is represented by Formula (Ib):

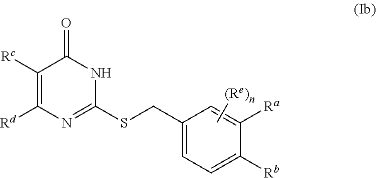

or a pharmaceutically acceptable salt thereof, wherein n is 0, 1, 2, or 3.

Embodiment I-62. The use of any one of Embodiment I-59 to I-61,
wherein:
one of R^{a} and R is hydrogen and the other is CO₂R^{x}, CH₂CO₂R^{x}, tetrazole, or oxadiazolone;
R^{c} is halogen, —CN, —OR^{x}, or C₁-C₆ alkyl;
R^{d} is methyl, optionally substituted 5- to 10-membered aryl, optionally substituted 5- or 6-membered heteroaryl, or optionally substituted 5- or 6-membered carbocycle; and
R^{x} is hydrogen or C₁-C₆ alkyl;
each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN;
each R^{y} and R^{z} is independently hydrogen, C₁-C₆ alkyl, or C₁-C₆ haloalkyl; and
n is 0, 1, 2, or 3;
with the proviso that R^{c} is not hydrogen or —CN when R^{d} is optionally substituted phenyl and that R^{c} is not —CN when R^{d} is 2-furyl.

Embodiment I-63. The use of any one of Embodiments I-59 to I-61, wherein the compound is represented by Formula (II):

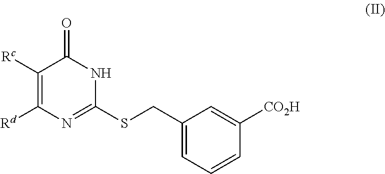

or a pharmaceutically acceptable salt thereof.

Embodiment I-64. The use of Embodiment I-63, wherein R^{c} is halogen, —CN, —OR^{x}, or C₁-C₆ alkyl, R^{d} is methyl, optionally substituted 5- to 10-membered aryl, optionally substituted 5- or 6-membered heteroaryl, or optionally substituted 5- or 6-membered carbocycle, and R^{x} is hydrogen or C₁-C₆ alkyl.

Embodiment I-65. The use of any one of Embodiments I-59 to I-64, wherein R^{c} is —CN or halogen.

Embodiment I-66. The use of any one of Embodiments I-59 to I-65, wherein R^{d} is methyl, cyclohexyl, pyridinyl, thiazolyl, phenyl, or thienyl.

Embodiment I-67. The use of any one of Embodiments I-59 to I-65, wherein R^{d} is methyl, cyclohexyl, pyridinyl, thiazolyl, thienyl, or optionally substituted phenyl.

Embodiment I-68. The use of any one of Embodiments I-59 to I-62, wherein R^{a} is hydrogen, CH₂CO₂H, tetrazole, or oxadiazolone (1,2,4-oxadiazol-5(4H)-one).

Embodiment I-69. The use of any one of Embodiments I-59 to I-62, wherein R^{b} is hydrogen, CH₂CO₂H, tetrazole, or oxadiazolone (1,2,4-oxadiazol-5(4H)-one).

Embodiment I-70. The use of any one of Embodiments I-59 to I-62, wherein n is 0.

Embodiment I-71. The use of Embodiment I-59, wherein the compound is selected from the group consisting of

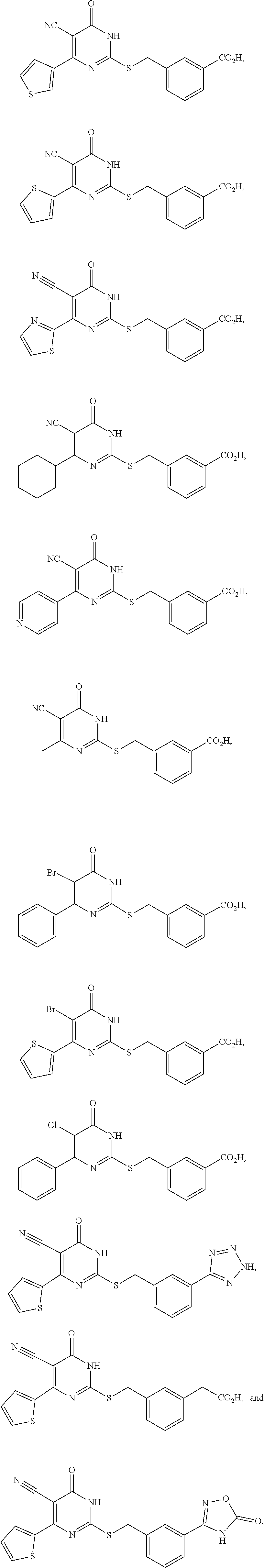

or a pharmaceutically acceptable salt thereof.

Embodiment I-72. The use of Embodiment I-59, wherein the compound is selected from the group consisting of

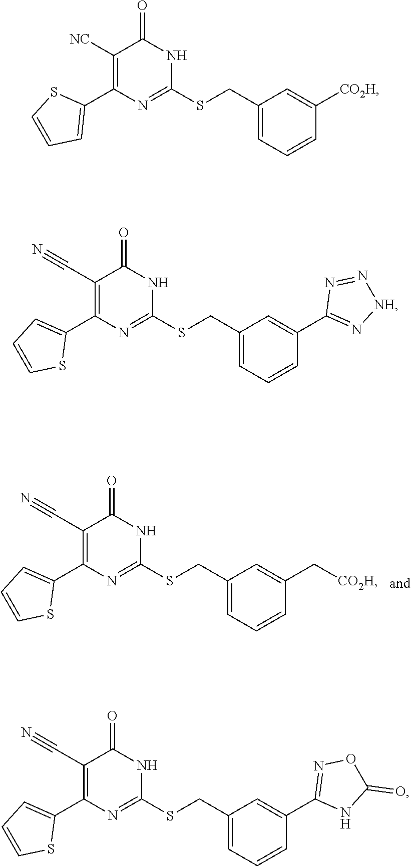 [SMILES: N#Cc1c(-c2cccs2)nc(SCc2cccc(C(=O)O)c2)[nH]c1=O ; N#Cc1c(-c2cccs2)nc(SCc2cccc(-c3nn[nH]n3)c2)[nH]c1=O ; N#Cc1c(-c2cccs2)nc(SCc2cccc(CC(=O)O)c2)[nH]c1=O ; "and" ; N#Cc1c(-c2cccs2)nc(SCc2cccc(-c3noc(=O)[nH]3)c2)[nH]c1=O]

or a pharmaceutically acceptable salt thereof.

Embodiment I-73. The use of Embodiment I-59, wherein the compound is selected from the group consisting of

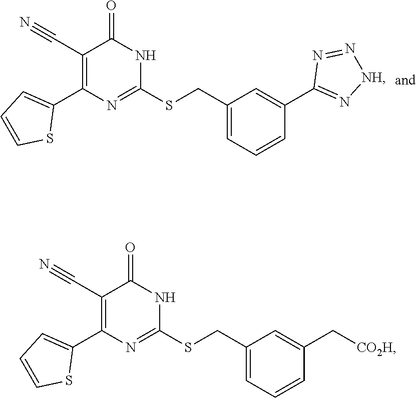 [SMILES: N#Cc1c(-c2cccs2)nc(SCc2cccc(-c3nn[nH]n3)c2)[nH]c1=O ; "and" ; N#Cc1c(-c2cccs2)nc(SCc2cccc(CC(=O)O)c2)[nH]c1=O]

or a pharmaceutically acceptable salt thereof.

Embodiment I-74. The use of Embodiment I-59, wherein the compound is

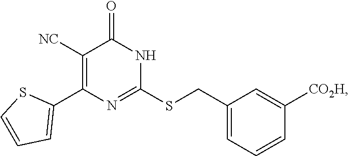 [SMILES: N#Cc1c(-c2cccs2)nc(SCc2cccc(C(=O)O)c2)[nH]c1=O]

or a pharmaceutically acceptable salt thereof.

Embodiment I-75. The use of Embodiment I-59, wherein the compound is

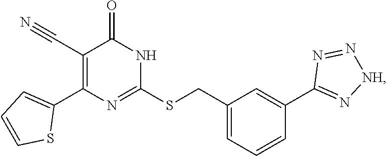 [SMILES: N#Cc1c(-c2cccs2)nc(SCc2cccc(-c3nn[nH]n3)c2)[nH]c1=O]

or a pharmaceutically acceptable salt thereof.

Embodiment I-76. The use of Embodiment I-59, wherein the compound is

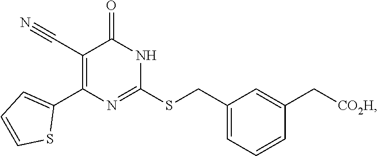 [SMILES: N#Cc1c(-c2cccs2)nc(SCc2cccc(CC(=O)O)c2)[nH]c1=O]

or a pharmaceutically acceptable salt thereof.

Embodiment I-77. The use of Embodiment I-59, wherein the compound is

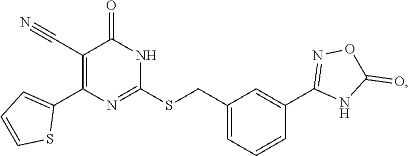 [SMILES: N#Cc1c(-c2cccs2)nc(SCc2cccc(-c3noc(=O)[nH]3)c2)[nH]c1=O]

or a pharmaceutically acceptable salt thereof.

Embodiment I-78. The use of any one of Embodiments I-59 to I-77, wherein the acute inflammatory condition is a systemic inflammatory condition.

Embodiment I-79. The use of any one of Embodiments I-59 to I-77, wherein the acute inflammatory condition is an organ-specific condition.

Embodiment I-80. The use of any one of Embodiments I-59 to I-77, wherein the acute inflammatory condition is a cytokine storm or hypercytokinemia, systemic inflammatory response syndrome (SIRS), graft versus host disease (GVHD), acute respiratory distress syndrome (ARDS), severe acute respiratory distress syndrome (SARS), catastrophic anti-phospholipid syndrome, viral infections, bacterial infections, fungal infections, influenza, pneumonia, shock, or sepsis.

Embodiment I-81. The use of any one of Embodiments I-59 to I-77, wherein the acute inflammatory condition is acute pancreatitis, hepatitis, respiratory condition, or enterocolitis.

Embodiment I-82. The use of any one of Embodiments I-59 to I-81, wherein a pro-inflammatory cytokine is reduced or an anti-inflammatory cytokine is increased.

Embodiment I-83. The use of Embodiment I-82, wherein the pro-inflammatory cytokine is IL-1β, IL-6, IL-18, TNF-α, or TGF-β.

Embodiment I-84. The use of Embodiment I-82, wherein pro-inflammatory cytokine is MCP-1, TNF-α, or IL-10.

Embodiment I-85. The use of Embodiment I-82, wherein pro-inflammatory cytokine is IL-6.

Embodiment I-86. The use of Embodiment I-82, wherein the anti-inflammatory cytokine is IL-10.

Embodiment I-87. The use of any one of Embodiments I-59 to I-81, wherein expression of sirtuin-1 modulated genes sod2, tfam, dda1 genes are increased in the liver.

Embodiment I-88. Use of a compound represented by Formula (I):

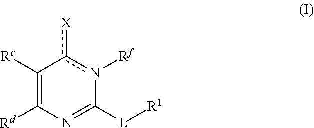

or a pharmaceutically acceptable salt or tautomer thereof,
wherein:
X is O or OH;
L is —(CH₂)ₘCH₂CH₂—, —(CH₂)ₘY(CH₂)ₚ—, —(CH₂)ₘC(O)(CH₂)ₚ—, —(CH₂)ₘC(O)O(CH₂)ₚ—, —(CH₂)ₘC(O)NR²(CH₂)ₚ—, or —(CH₂)ₘNR²C(O)(CH₂)ₚ;
Y is O, N or S(O)q;
R¹ is C₆-C₁₀ aryl or heteroaryl, wherein the aryl and heteroaryl are substituted with R^{a} and R^{b}, and optionally substituted with one or more R^{e}.
R² is H or C₁-C₆ alkyl;
one of R^{a} and R^{b} is hydrogen and the other is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣdihydrotetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl;
R^{c} is H, C₁-C₆ alkyl, C₁-C₆ haloalkyl, halogen, —CN, —OR^{x}, —CO₂R^{x}, or NO₂;
R^{d} is methyl, optionally substituted 5- to 10-membered aryl, optionally substituted 5- or 6-membered heteroaryl, or optionally substituted 5- or 6-membered carbocycle;
each R^{x} is independently at each occurrence hydrogen or C₁-C₆ alkyl;
each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN;
R^{f} is H or absent;
each R^{y} and R^{z} is independently hydrogen, C₁-C₆ alkyl, or C₁-C₆ haloalkyl;
each m and p independently is 0, 1, or 2, wherein m+p<3;
q is 0, 1, or 2;
r is 0 or 1; and
the dotted line is an optional double bond;
with the proviso that R^{c} is not hydrogen or —CN when X is O, L is —SCH₂— and R^{d} is optionally substituted phenyl, R^{c} is not C₁-C₆ alkyl when X is O, L is —SCH₂— and R^{d} is methyl, and that R^{c} is not —CN when X is O, L is —SCH₂— and R^{d} is 2-furyl;
in the manufacture of a medicament for treating an acute inflammatory condition.

Embodiment I-89. The use of Embodiment I-88, wherein the compound is represented by Formula (Ia)

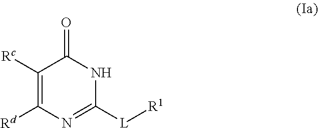

or a pharmaceutically acceptable salt, or tautomer thereof.

Embodiment I-90. The use of Embodiment I-88 or I-89, wherein the compound is represented by Formula (Ib):

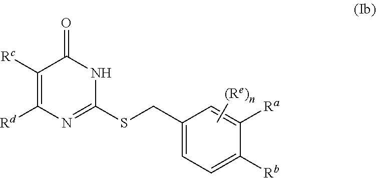

or a pharmaceutically acceptable salt thereof, wherein n is 0, 1, 2, or 3.

Embodiment I-91. The use of any one of Embodiments I-88 to I-90,
wherein:
one of R^{a} and R is hydrogen and the other is CO₂R^{x}, CH₂CO₂R^{x}, tetrazole, or oxadiazolone;
R^{c} is halogen, —CN, —OR^{x}, or C₁-C₆ alkyl;
R^{d} is methyl, optionally substituted 5- to 10-membered aryl, optionally substituted 5- or 6-membered heteroaryl, or optionally substituted 5- or 6-membered carbocycle; and
R^{x} is hydrogen or C₁-C₆ alkyl;
each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN;
each R^{y} and R^{z} is independently hydrogen, C₁-C₆ alkyl, or C₁-C₆ haloalkyl; and
n is 0, 1, 2, or 3;
with the proviso that R^{c} is not hydrogen or —CN when R^{d} is optionally substituted phenyl and that R^{c} is not —CN when R^{d} is 2-furyl.

Embodiment I-92. The use of any one of Embodiments I-88 to I-90, wherein the compound is represented by Formula (II):

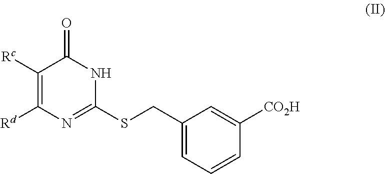

or a pharmaceutically acceptable salt thereof.

Embodiment I-93. The use of Embodiment I-92, wherein R^{c} is halogen, —CN, —OR^{x}, or C₁-C₆ alkyl, R^{d} is methyl, optionally substituted 5- to 10-membered aryl, optionally substituted 5- or 6-membered heteroaryl, or optionally substituted 5- or 6-membered carbocycle, and R^{x} is hydrogen or C₁-C₆ alkyl.

Embodiment I-94. The use of any one of Embodiments I-88 to I-93, wherein R^{c} is —CN or halogen.

Embodiment I-95. The use of any one of Embodiments I-88 to I-94, wherein R^{d} is methyl, cyclohexyl, pyridinyl, thiazolyl, phenyl, or thienyl.

Embodiment I-96. The use of any one of Embodiments I-88 to I-94, wherein R^{d} is methyl, cyclohexyl, pyridinyl, thiazolyl, thienyl, or optionally substituted phenyl.

Embodiment I-97. The use of any one of Embodiments I-88 to I-91, wherein R^{a} is hydrogen, CH₂CO₂H, tetrazole, or oxadiazolone (1,2,4-oxadiazol-5(4H)-one).

Embodiment I-98. The use of any one of Embodiments I-88 to I-91, wherein R^{b} is hydrogen, CH₂CO₂H, tetrazole, or oxadiazolone (1,2,4-oxadiazol-5(4H)-one).

Embodiment I-99. The use of any one of Embodiments I-88 to I-91, wherein n is 0.

Embodiment I-100. The use of Embodiment I-88, wherein the compound is selected from the group consisting of

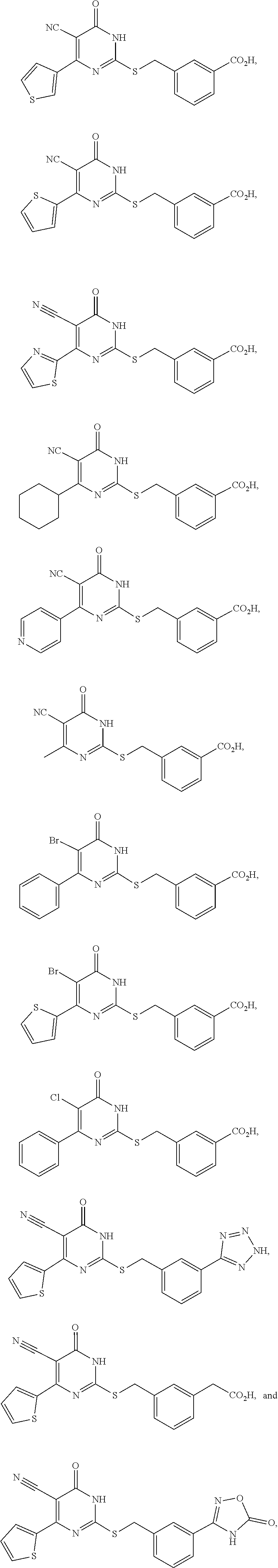

or a pharmaceutically acceptable salt thereof.

Embodiment I-101. The use of Embodiment I-88, wherein the compound is selected from the group consisting of

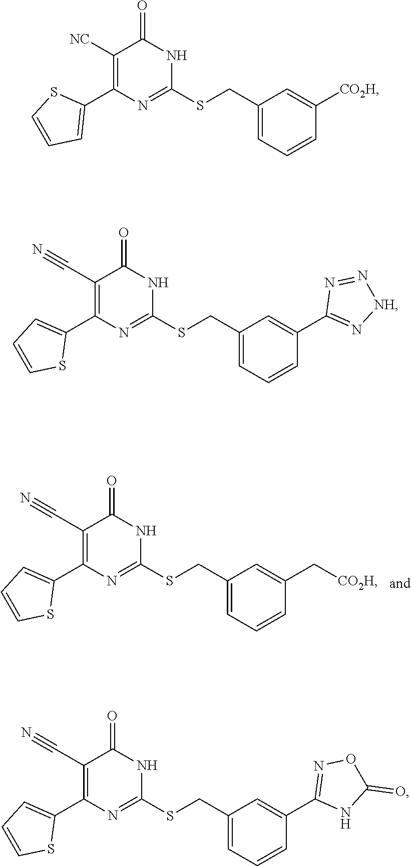 [SMILES: N#Cc1c(-c2cccs2)nc(SCc2cccc(C(=O)O)c2)[nH]c1=O ; N#Cc1c(-c2cccs2)nc(SCc2cccc(-c3nn[nH]n3)c2)[nH]c1=O ; N#Cc1c(-c2cccs2)nc(SCc2cccc(CC(=O)O)c2)[nH]c1=O ; "and" ; N#Cc1c(-c2cccs2)nc(SCc2cccc(-c3noc(=O)[nH]3)c2)[nH]c1=O]

or a pharmaceutically acceptable salt thereof.

Embodiment I-102. The use of Embodiment I-88, wherein the compound is selected from the group consisting of

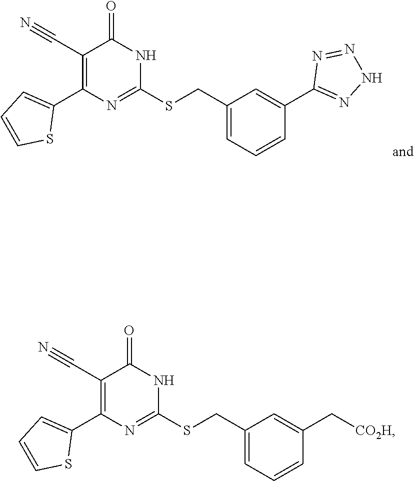

or a pharmaceutically acceptable salt thereof.

Embodiment I-103. The use of Embodiment I-88, wherein the compound is

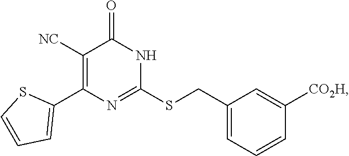 [SMILES: N#Cc1c(-c2cccs2)nc(SCc2cccc(C(=O)O)c2)[nH]c1=O]

or a pharmaceutically acceptable salt thereof.

Embodiment I-104. The use of Embodiment I-88, wherein the compound is

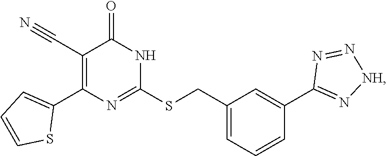

or a pharmaceutically acceptable salt thereof.

Embodiment I-105. The use of Embodiment I-88, wherein the compound is

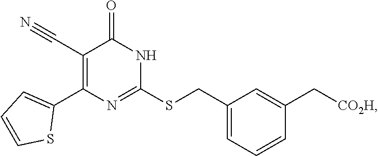 [SMILES: N#Cc1c(-c2cccs2)nc(SCc2cccc(CC(=O)O)c2)[nH]c1=O]

or a pharmaceutically acceptable salt thereof.

Embodiment I-106. The use of Embodiment I-88, wherein the compound is

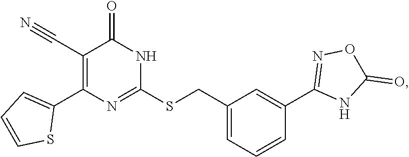 [SMILES: N#Cc1c(-c2cccs2)nc(SCc2cccc(-c3noc(=O)[nH]3)c2)[nH]c1=O]

or a pharmaceutically acceptable salt thereof.

Embodiment I-107. The use of any one of Embodiments I-88 to I-106, wherein the acute inflammatory condition is a systemic inflammatory condition.

Embodiment I-108. The use of any one of Embodiments I-88 to I-106, wherein the acute inflammatory condition is an organ-specific condition.

Embodiment I-109. The use of any one of Embodiments I-88 to I-106, wherein the acute inflammatory condition is a cytokine storm or hypercytokinemia, systemic inflammatory response syndrome (SIRS), graft versus host disease (GVHD), acute respiratory distress syndrome (ARDS), severe acute respiratory distress syndrome (SARS), catastrophic anti-phospholipid syndrome, viral infections, bacterial infections, fungal infections, influenza, pneumonia, shock, or sepsis.

Embodiment I-110. The use of any one of Embodiments I-88 to I-106, wherein the acute inflammatory condition is acute pancreatitis, hepatitis, respiratory condition, or enterocolitis.

Embodiment I-111. The use of any one of Embodiments I-88 to I-110, wherein a pro-inflammatory cytokine is reduced or an anti-inflammatory cytokine is increased.

Embodiment I-112. The use of Embodiment I-111, wherein the pro-inflammatory cytokine is IL-1β, IL-6, IL-18, TNF-α, or TGF-β.

Embodiment I-113. The use of Embodiment I-111, wherein pro-inflammatory cytokine is MCP-1, TNF-α, or IL-1β.

Embodiment I-114. The use of Embodiment I-111, wherein pro-inflammatory cytokine is IL-6.

Embodiment I-115. The use of Embodiment I-111, wherein the anti-inflammatory cytokine is IL-10.

Embodiment I-116. The use of any one of Embodiments I-88 to I-110, wherein expression of sirtuin-1 modulated genes sod2, tfam, dda1 genes are increased in the liver.

All percentages and ratios used herein, unless otherwise indicated, are by weight. Other features and advantages of the present disclosure will become apparent from the different examples. The provided examples illustrate different components and methodology useful in practicing the present disclosure. Generally speaking, the disclosure extends to any novel one, or any novel combination, of the features disclosed in this specification (including the accompanying claims and drawings). The examples do not limit the claimed disclosure. Thus, features, integers, characteristics, compounds or chemical moieties described in conjunction with a particular aspect, embodiment or example of the disclosure are to be understood to be applicable to any other aspect, embodiment or example described herein, unless incompatible therewith. Based on the present disclosure the skilled artisan can identify and employ other components and methodology useful for practicing the present disclosure. Moreover, unless stated otherwise, any feature disclosed herein may be replaced by an alternative feature serving the same or a similar purpose.

The Disclosure will now be described by way of example only with reference to the Examples below:

### EXEMPLIFICATION

I. COMPOUND PREPARATION

### General Methods and Materials

All chemicals were purchased from Sigma-Aldrich, Alfa Aesar. ¹H NMR spectra were recorded at 200 and 400 MHz and ¹³C NMR spectra were recorded at 100.6 and 50.3 MHz by using deuterated solvents indicated below. TLC were performed on aluminium backed silica plates (silica gel 60 F254). All the reactions were performed under nitrogen atmosphere using distilled solvents. All tested compounds were found to have >95% purity determined by HPLC analysis. HPLC-grade water was obtained from a tandem Milli-Ro/Milli-Q apparatus. The analytical HPLC measurements were made on a Shimadzu LC-20AProminence equipped with a CBM-20A communication bus module, two LC-20AD dual piston pumps, a SPD-M20A photodiode array detector and a Rheodyne 7725i injector with a 20 μL stainless steel loop.

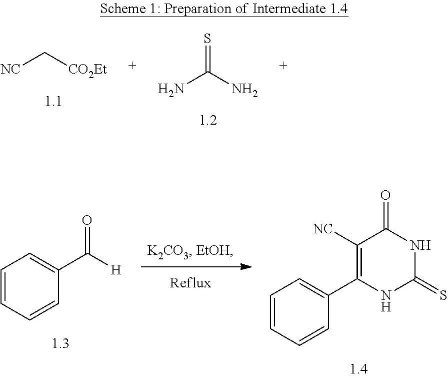

### Example 1: Preparation of Intermediate 1.4

To a stirred solution of compounds 1.1 (0.52 ml, 4.9 mmol), 1.2 (372 mg, 4.9 mmol) and 1.3 (0.5 mL, 0.83 mL, 4.9 mmol) in ethanol (25 mL) was added K₂CO₃ (812 mg, 5.88 mmol). Stirring was continued at reflux overnight. The pale yellow solid was collected after cooling, taken up with boiling water and filtered again. The aqueous phase was acidified to pH 5 with AcOH (15 drops), the precipitate was filtered and dried under vacuum. The title compound 1.4 was obtained as pale yellow solid (500 g, 2.18 mmol). Yield 44%.

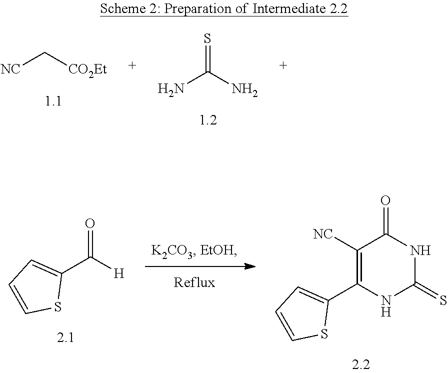

### Example 2: Preparation of Intermediate 2.2

To a stirred solution of compounds 1.1 (0.96 g, 8.8 mmol), 1.2 (672 mg, 8.8 mmol) and 2.1 (1 g, 0.83 mL) in ethanol (55 mL) was added K₂CO₃ (1.57 g, 11.44 mmol). Stirring was continued at reflux overnight. The yellowish solid was collected after cooling, taken up with hot water and filtered again. The aqueous phase was acidified to pH 1, the precipitate was filtered and dried under vacuum. The title compound 2.2 was obtained as yellowish solid (1 g, 4.25 mmol). Yield 49%. ¹H NMR (200 MHz, DMSO) δ 7.22 (m, 1H), 7.68 (m, 1H), 7.85 (d, J=4.8 Hz, 1H), 8.05 (s, 1H).

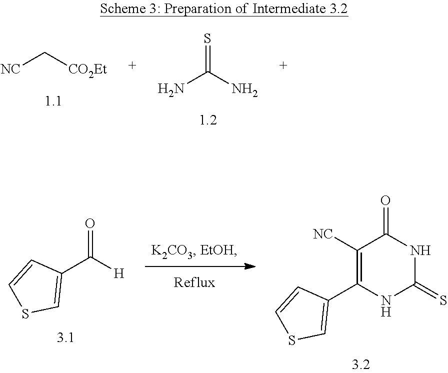

### Example 3: Preparation of Intermediate 3.2

To a stirred solution of compounds 1.1 (0.96 mL, 8.8 mmol), 1.2 (672 mg, 8.8 mmol) and 3.1 (1 g, 1.29 mL) in ethanol (55 mL) was added K₂CO₃ (1.57 g, 11.44 mmol). Stirring was continued at reflux overnight. The yellowish solid was collected after cooling, taken up with hot water and filtered again. The aqueous phase was acidified to pH 1, the precipitate was filtered and dried under vacuum. The title compound 3.2 was obtained as yellowish solid (1 g, 4.25 mmol). Yield 49%.

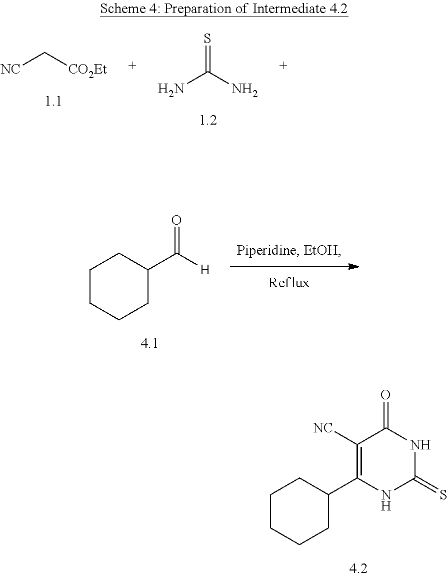

### Example 4: Preparation of Intermediate 4.2

To a stirred solution of compounds 1.1 (1.42 mL, 13.37 mmol), 1.2 (1.01 g, 13.3 mmol) and 4.1 (1.62 mL, 13.3 mL) in ethanol (50 mL) was added piperidine (2.64 mL, 26.7 mmol). Stirring was continued at reflux overnight. The solid was collected after cooling, taken up with hot water and filtered again. The aqueous phase was acidified to pH1 and extracted with EtOAc (3×25 mL). The organic phase was washed with brine and dried over Na₂SO₄. The crude of reaction was purified by flash chromatography (CHCl₃/MeOH as gradient, from 0 to 2% for product), affording the title compound 4.2 (930 mg, 3.95 mmol) as white solid. Yield 30%.

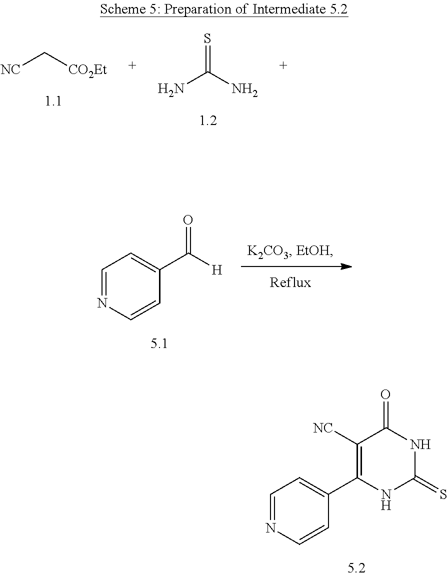

### Example 5: Preparation of Intermediate 5.2

To a stirred solution of compound s1.1 (0.49 mL, 4.67 mmol), 1.2 (355 mg, 4.67 mmol) and 5.1 (0.44 mL, 4.67 mmol) in ethanol (25 mL) was added K₂CO₃ (773 mg, 5.6 mmol). Stirring was continued at reflux overnight. The white solid was collected after cooling, dried under vacuum and used for the next step without further purification. The title compound 5.2 was obtained as white solid (300 mg, 1.3 mmol). Yield 29%. ¹H NMR (400 MHz, DMSO) δ 7.64 (d, J=4.7 Hz, 2H), 8.78 (d, J=4.7 Hz, 2H), 12.98 (s, 1H).

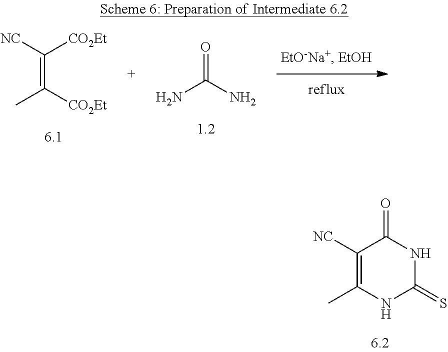

### Example 6: Preparation of Intermediate 6.2

To a stirred solution NaOEt (1.02 mL, 2.73 mmol) in EtOH abs (20 mL) were added compounds 6.1 (500 mg, 2.73 mmol) and 1.2 (207 mg, 2.73 mmol). Stirring was continued at reflux 4 h. The volatiles were removed under vacuum. The crude of reaction was taken up with water and acidified with AcOH. The precipitate was collected dissolved in water, washed with a mixture of CHCl₃ and MeOH. The aqueous phase was extracted with EtOAc (3×20 mL). The collected organic phase was washed with brine, dried over Na₂SO₄. The title compound 6.2 was obtained as white solid (250 mg, 1.49 mmol). Yield 55%.

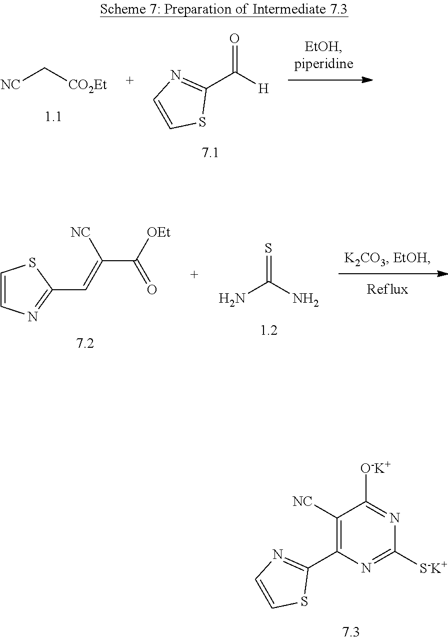

### Example 7a: Preparation of Intermediate 7.2

To a stirred solution of compounds 1.1 (0.14 mL, 1.3 mmol) and 7.1 (150 mg, 1.3 mmol) in EtOH (5 mL) was added piperidine (1 drop). Stirring was continued at room temperature overnight. The solvent was removed under vacuum. The crude of reaction was purified by flash chromatography affording the title compound 7.2 (160 mg, 0.77 mmol) as yellowish solid. Yield 58%.

### Example 7b: Preparation of Intermediate 7.3

To a stirred suspension of compounds 7.2 (150 mg, 0.72 mmol) and compound 1.2 (55 mg, 0.72 mmol) in EtOH (5 mL) was added K₂CO₃ (99 mg, 0.72 mmol). Stirring was continued at reflux overnight. The white precipitate was collected and used as well for the next step without further purification. The title compound 7.3 (150 mg, 0.48 mmol) was obtained as yellowish solid as di-potassium salt. Yield 67%.

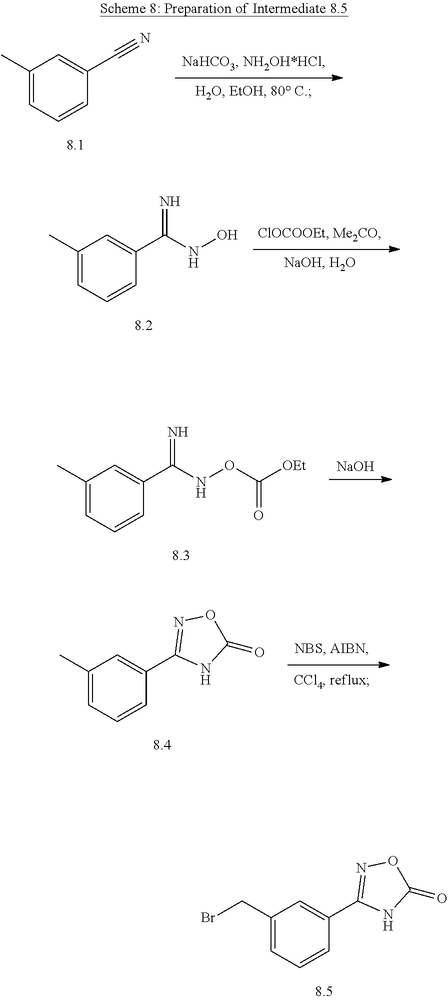

### Example 8a: Preparation of Intermediate 8.2

To a stirred solution of NH₂OH*HCl and NaHCO₃ in water (7 mL) was gradually added a solution of m-tolunitrile (8.1) (2 mL, 17.0 mmol) in EtOH (13.3 mL). Stirring was continued at 80° C. for 4 h. The volatiles were removed under vacuum. The crude of reaction was taken up with water, extracted with EtOAc (3×25 mL). The organic phase were collected, washed with brine and dried over Na₂SO₄ affording the title compound 8.2 (1.5 g, 9 mmol) as white solid. Yield 59%.

### Example 8b: Preparation of Intermediate 8.3

To a solution of compound 8.2 (1 g, 6 mmol) in dry acetone (5 mL), was added dropwise at 0° C. EtOCOCl (0.63 mL, 6.6 mmol). Stirring was continued at this temperature for 1 h. Then a 5% NaOH solution was added to the mixture. Stirring was continued for additional 1 h. The solvent was removed under vacuum. The crude of reaction was poured in water, extracted with EtOAc (3×50 mL). The collected organic phase was washed with brine, dried over Na₂SO₄. The title compound 8.3 (600 mg, 2.7 mmol) was obtained as white solid. Yield 45%.

### Example 8c: Preparation of Intermediate 8.4

To a solution of compound 8.3 (300 mg, 1.35 mmol) in EtOH abs (5 mL) was added sodium (50 mg) portion wise. Stirring was continued at room temperature for additional 4 h. The reaction was quenched by the addition of MeOH. The solvent was removed under reduced pressure and the crude was purified by flash chromatography. The title compound 8.4 (150 mg, 0.85 mmol) was obtained as white solid. Yield 63%.

### Example 8d: Preparation of Intermediate 8.5

To a suspension of compound 8.4 (326 mg, 1.85 mmol) in CCl₄ (10 mL) was added AIBN (60.7 mg, 0.37 mmol) and NBS (493 mg, 2.77 mmol). Stirring was continued at reflux overnight. The solvent was removed under reduced pressure. The reaction was taken up with water, extracted with EtOAc (3×20 mL) washed with brine and dried over Na₂SO₄. The crude was purified by flash chromatography, eluting with Petroleum ether (Pet. Ether)/EtOAc (30% for product) affording the title compound 8.5 (280 mg, 1.09 mmol) was obtained as white solid. Yield 59%.

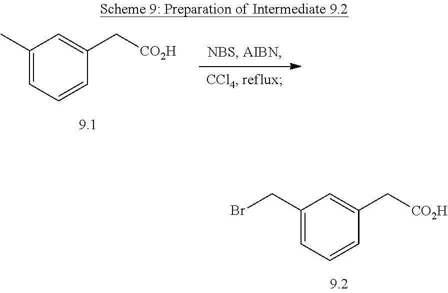

### Example 9: Preparation of Intermediate 9.2

To a suspension of compound 9.1 (750 mg, 5 mmol) in CCl₄ (15 mL) was added AIBN (41 mg, 0.25 mmol) and NBS (933.7 mg, 5.24 mmol). Stirring was continued at reflux overnight. The solvent was removed under reduced pressure. The reaction was taken up with water, extracted with EtOAc (3×20 mL) washed with brine and dried over Na₂SO₄. The crude was purified by flash chromatography, eluting with CH₂Cl₂/MeOH (3% for product) affording the title compound 9.2 (800 mg, 3.49 mmol) as white solid. Yield 70%.

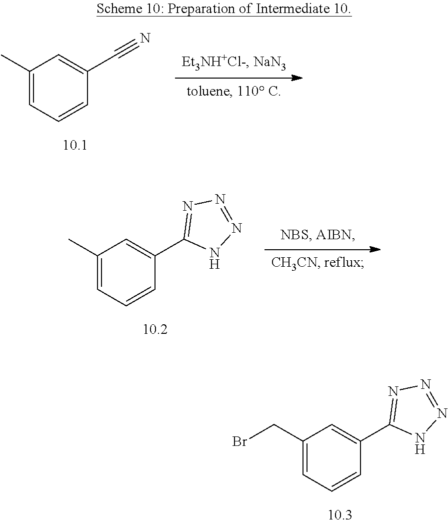

### Example 10a: Preparation of Intermediate 10.2

A mixture of compound 10.1 (1.02 mL, 8.54 mmol), NaN₃ (832 mg, 12.8 mmol) and Et₃N*HCl (1.76 g, 12.8 mmol) was heated at reflux 4 h. The solvent was removed under vacuum. The crude was poured in water, acidified to pH 1 with 3N HCl and extracted with EtOAc (3×20 mL). The organic phase was washed with brine, dried over Na₂SO₄ and concentrated under reduced pressure. The title compound 10.2 (1.22 g, 7.6 mmol) was obtained as white solid. Yield 89%.

### Example 10b: Preparation of Intermediate 10.3

To a suspension of compound 10.2 (300 mg, 1.87 mmol) in CH₃CN (15 mL) was added AIBN (31 mg, 0.18 mmol) and NBS (333 mg, 1.87 mmol). Stirring was continued at reflux overnight. The solvent was removed under reduced pressure. The reaction was taken up with water, extracted with EtOAc (3×20 mL) washed with brine and dried over Na₂SO₄. The crude was purified by flash chromatography, eluting with CH₂Cl₂/MeOH (7% for product) affording the title compound 10.3 (150 mg, 0.62 mmol) as light yellow solid. Yield 34%.

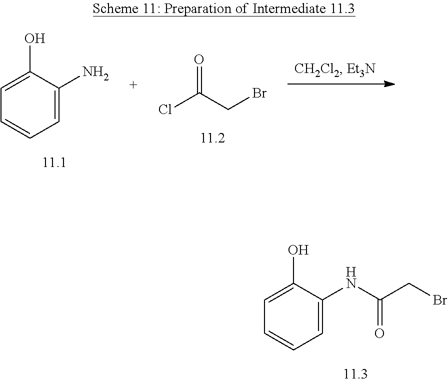

### Example 11: Preparation of Intermediate 11.3

To a solution of compound 11.1 (2.5 g, 23 mmol) in CH₂Cl₂ (25 mL) was added pyridine (1.63 mL, 20.3 mmol) and compound 11.2 (1.68 mL, 20.3 mmol). Stirring was continued at room temperature overnight. The solvent was removed under reduced pressure. The reaction was taken up with water, extracted with CH₂Cl₂ (3×30 mL) washed with brine and dried over Na₂SO₄. The crude was purified by flash chromatography, eluting with Pet. Ether/EtOAc (25% for product) affording the title compound 11.3 (735 mg, 3.19 mmol) as brownish solid. Yield 14%.

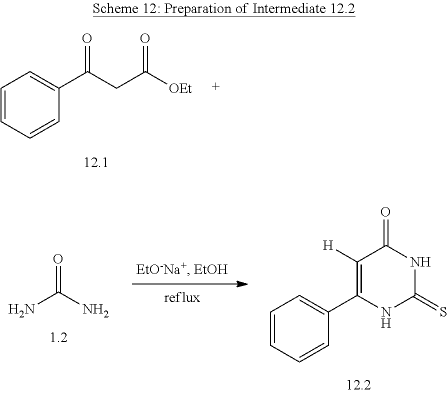

### Example 12: Preparation of Intermediate 12.2

To a solution of compound 12.1 (2 g, 10.41 mmol) in EtOH (15 mL) was added EtONa (7 mL, 18.7 mmol) and compound 1.2 (1.18 g, 15.61 mmol). Stirring was continued at reflux overnight. The solvent was removed under reduced pressure. The reaction was taken up with water, acidified to pH 3, extracted with EtOAc (3×20 mL) washed with brine and dried over Na₂SO₄. The crude was purified by flash chromatography, eluting with CH₂Cl₂/MeOH (2.5% for product) affording the title compound 12.2 (500 mg, 2.44 mmol) as white solid. Yield 24%.

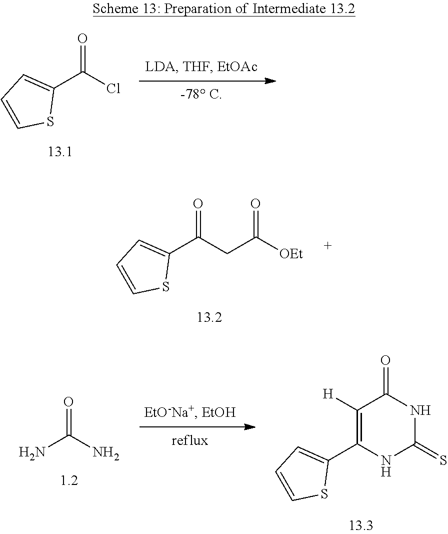

### Example 13a: Preparation of Intermediate 13.2

To a stirred solution of DIPA (7.6 mL, 54 mmol) in THF (53 mL) was added n-BuLi (21.6 mL) at 0° C. Stirring was continued at this temperature 10 minutes. The mixture was then cooled to −78° C. and EtOAc (2.4 mL, 27 mmol) was added dropwise. Stirring was continued at this temperature 30 minutes. After that, a solution of compound 13.1 (3 mL, 27 mmol) in THF (20 mL) was added dropwise. The reaction was allowed to warm to room temperature and was stirred overnight. The crude of reaction was poured in water and extracted with EtOAc (3×30 mL). The collected organic phase were washed with brine, dried over Na₂SO₄ and concentrated under vacuum. The title compound 13.2 was obtained as brownish oil (4.8 g, 24.3 mmol). Yield 90%.

### Example 13b: Preparation of Intermediate 13.3

To a solution of intermediate 13.2 (2 g, 10 mmol) in EtOH (15 mL) was added EtONa (21% wt/wt in EtOH) (7.5 mL, 20 mmol) and compound 1.2 (1.15 g, 15.1 mmol). Stirring was continued at reflux overnight. The solvent was removed under reduced pressure. The reaction was taken up with water. At pH 10 was recovered unreacted starting material. The mixture was then acidified to pH 5, extracted with EtOAc (3×20 mL) washed with brine and dried over Na₂SO₄. The crude was purified by flash chromatography, eluting with CH₂Cl₂/MeOH (7% for product) affording the title compound 13.3 (435 mg, 2.06 mmol) as yellowish solid. Yield 21%.

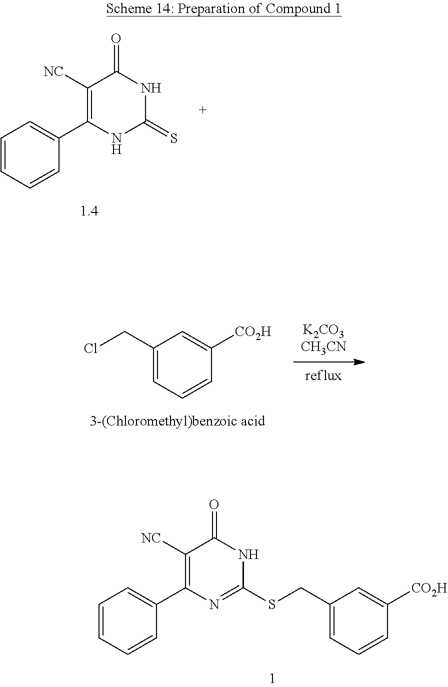 [SMILES: N#Cc1c(-c2ccccc2)[nH]c(=S)[nH]c1=O ; O=C(O)c1cccc(CCl)c1 ; "K2CO3CH3CN" ; "reflux" ; N#Cc1c(-c2ccccc2)nc(SCc2cccc(C(=O)O)c2)[nH]c1=O]

### Example 14: Preparation of Compound 1

To a stirred suspension of intermediate 1.4 (1.6 g, 6.98 mmol) and K₂CO₃ (2.88 g, 20.9 mmol) in CH₃CN (80 mL) was added 3-(chloromethyl)benzoic acid (1.19 g, 6.98 mmol). Stirring was continued overnight at reflux. The volatiles were removed under vacuo. The crude was taken up with water, acidified to pH 5 and washed with EtOAc to remove impurities. Then the pH was adjusted to ¾ and the mixture was extracted with EtOAc (3×50 mL). Titration with hot acetone afforded compound 1 (936 mg, 2.78 mmol) as yellowish solid. Yield 40%. ¹H NMR (400 MHz, DMSO) δ 4.58 (s, 2H), 7.44 (t, J=7.5 Hz, 1H), 7.54-7.61 (m, 3H), 7.67 (d, J=7.1 Hz, 1H), 7.83 (d, J=7.5 Hz, 1H), 7.91 (d, J=7.27 Hz, 2H), 8.04 (s, 1H), 13 (s, 1H); ¹³C NMR (100 MHz, DMSO) δ 33.5, 93.2, 115.6, 128.2, 128.4, 128.4, 128.5, 128.5, 128.6, 129.7, 130.8, 131.5, 133.3, 135.1, 137.4, 165.4, 166.8, 167.3. HPLC. 96.3%

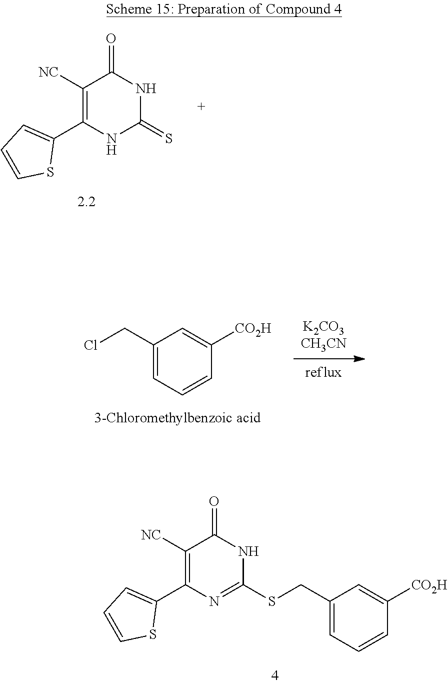 [SMILES: N#Cc1c(-c2cccs2)[nH]c(=S)[nH]c1=O ; O=C(O)c1cccc(CCl)c1 ; "K2CO3CH3CN" ; "reflux" ; N#Cc1c(-c2cccs2)nc(SCc2cccc(C(=O)O)c2)[nH]c1=O]

### Example 15: Preparation of Compound 4

To a stirred suspension of intermediate 2.2 (250 mg, 1.06 mmol) and K₂CO₃ (440 mg, 3.18 mmol) in CH₃CN (15 mL) was added 3-(chloromethyl)benzoic acid (180 mg, 1.06 mmol). Stirring was continued overnight at reflux. The volatiles were removed under vacuo. The crude was taken up with water, washed with EtOAc, acidified to pH 1 and extracted with EtOAc (3×50 mL). Titration with hot acetone afforded compound 4 (45 mg, 0.12 mmol) as yellowish solid. Yield 12%. ¹H NMR (400 MHz, DMSO) δ 4.62 (s, 2H), 7.33 (t, J=4.3 Hz, 1H), 7.44 (t, J=7.6 Hz, 1H), 7.72 (d, J=7.5 Hz, 1H), 7.82 (d, J=7.5 Hz, 1H), 8.05 (m, 2H), 8.26 (d, J=3.8 Hz, 1H), 12.99 (s, 1H); ¹³C NMR (100 MHz, DMSO) δ 33.9, 88.7, 116.5, 128.8, 129.3, 129.9, 130.2, 131.5, 132.1, 133.7, 135.4, 137.9, 139.7, 159.0, 161.2, 165.3, 167.4. HPLC: 97.2%

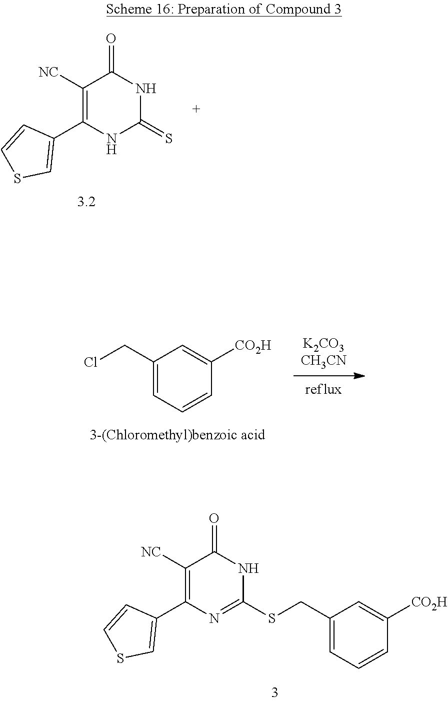 [SMILES: N#Cc1c(-c2ccsc2)[nH]c(=S)[nH]c1=O ; O=C(O)c1cccc(CCl)c1 ; "K2CO3CH3CN" ; "reflux" ; N#Cc1c(-c2ccsc2)nc(SCc2cccc(C(=O)O)c2)[nH]c1=O]

### Example 16: Preparation of Compound 3

To a stirred suspension of intermediate 3.2 (250 mg, 1.06 mmol) and K₂CO₃ (440 mg, 3.18 mmol) in CH₃CN (15 mL) was added 3-(chloromethyl)benzoic acid (180 mg, 1.06 mmol). Stirring was continued overnight at reflux. The volatiles were removed under vacuo. The crude was taken up with water, washed with EtOAc, acidified to pH 1 and extracted with EtOAc (3×50 mL). Titration with a mixture of Et₂O/acetone afforded compound 3 (260 mg, 0.7 mmol) as yellowish solid. Yield 70%. ¹H NMR (400 MHz, DMSO) δ 4.63 (s, 2H), 7.44 (t, J=7.6 Hz, 1H), 7.69 (d, J=7.7 Hz, 1H), 7.74 (dd, J=5 Hz, J=2.9 Hz, 1H), 7.84 (m, 2H), 8.05 (s, 1H), 8.58 (m, 1H), 13.0 (s, 1H); ¹³C NMR (100 MHz, DMSO) δ 35.3, 90.1, 118.0, 130.2, 130.7, 131.3, 131.6, 132.9, 133.5, 135.1, 136.8, 139.3, 141.1, 160.4, 162.7, 166.7, 168.8. HPLC: 95.0%

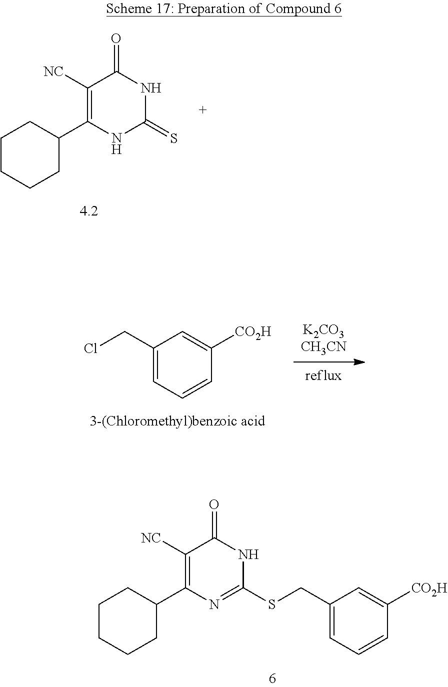 [SMILES: N#Cc1c(C2CCCCC2)[nH]c(=S)[nH]c1=O ; O=C(O)c1cccc(CCl)c1 ; "K2CO3CH3CN" ; "reflux" ; N#Cc1c(C2CCCCC2)nc(SCc2cccc(C(=O)O)c2)[nH]c1=O]

### Example 17: Preparation of Compound 6

To a stirred suspension of intermediate 4.2 (250 mg, 1.18 mmol) and K₂CO₃ (495 mg, 3.56 mmol) in CH₃CN (15 mL) was added 3-(chloromethyl)benzoic acid (202 mg, 1.18 mmol). Stirring was continued overnight at reflux. The volatiles were removed under vacuo. The crude was taken up with water, washed with EtOAc, acidified to pH 1 and extracted with EtOAc (3×50 mL). Titration with Et₂O afforded compound 6 (90 mg, 0.24 mmol) as white solid. Yield 21%. ¹H NMR (400 MHz, DMSO) δ 1.24 (m, 3H), 1.60 (m, 7H), 2.74 (m, 1H), 4.52 (s, 2H), 7.45 (t, J=7.18 Hz, 1H), 7.67 (d, J=6.83 Hz, 1H), 7.82 (d, J=7.17 Hz, 1H), 8.04 (s, 1H), 13.0 (s, 1H). ¹³C NMR (100 MHz, DMSO) δ 25.4, 25.7, 25.7, 30.3, 30.3, 33.8, 44.9, 94.1, 115.3, 128.6, 129.2, 130.1, 131.3, 133.6, 138.5, 161.1, 166.2, 167.4, 177.9. HPLC: 98.1%

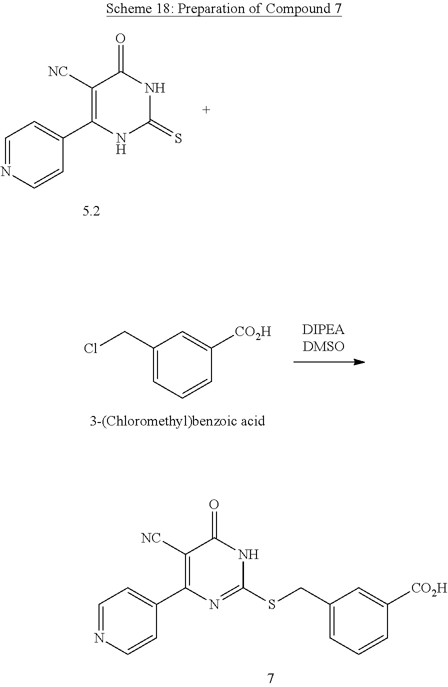 [SMILES: N#Cc1c(-c2ccncc2)[nH]c(=S)[nH]c1=O ; O=C(O)c1cccc(CCl)c1 ; "DIPEADMSO" ; N#Cc1c(-c2ccncc2)nc(SCc2cccc(C(=O)O)c2)[nH]c1=O]

### Example 18: Preparation of Compound 7

To a stirred suspension of intermediate 5.2 (220 mg, 0.95 mmol) and DIPEA (0.18 mL, 1.05 mmol) in DMSO (5 mL) was added 3-(chloromethyl)benzoic acid (178 mg, 1.05 mmol). Stirring was continued overnight at room temperature. The light yellow solid was collected, washed with crushed ice and water, and dried under vacuum. Trituration with hot EtOAc afforded compound 7 (180 mg, 0.49 mmol) as light yellow solid. Yield 53%. ¹H NMR (400 MHz, DMSO) δ 4.55 (s, 2H), 7.44 (m, 1H), 7.66 (d, J=6 Hz, 1H), 7.81 (m, 3H), 8.02 (s, 1H), 8.80 (s, 2H), 13.1 (s, 1H); ¹³C NMR (100 MHz, DMSO) δ 34.1, 94.8, 115.8, 122.8, 122.8, 128.7, 129.2, 130.4, 131.3, 133.9, 138.1, 143.1, 150.5, 150.5, 161.8, 165.7, 167.4, 167.4. HPLC: 95.1%

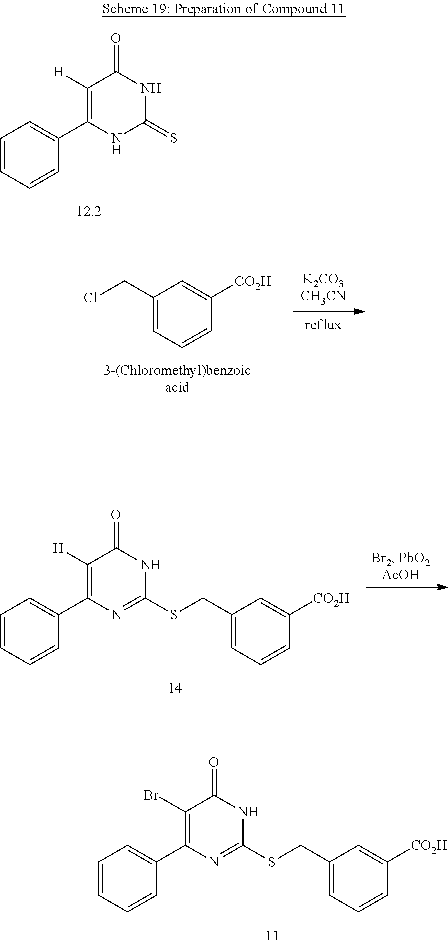

### Example 19a: Preparation of Compound 14

To a stirred suspension of intermediate 12.2 (100 mg, 0.43 mmol) and K₂CO₃ (178 mg, 1.29 mmol) in CH₃CN (15 mL) was added 3-(chloromethyl)benzoic acid (74 mg, 0.43 mmol). Stirring was continued overnight at reflux. The volatiles were removed under vacuo. The crude was taken up with water, washed with EtOAc, acidified to pH 3 and extracted with EtOAc (3×50 mL). Titration with a mixture of Et₂O/Acetone afforded compound 14 (30 mg, 0.088 mmol) as white solid. Yield 21%. ¹H NMR (400 MHz, DMSO) δ 4.59 (s, 2H), 6.69 (s, 1H), 7.41 (m, 1H), 7.46 (m, 3H), 7.71 (d, J=7.5 Hz, 1H), 7.81 (d, J=7.74 Hz, 1H), 8.06 (m, 3H), 12.85 (s, 2H). ¹³C NMR (100 MHz, DMSO) δ 33.8, 127.3, 127.3, 128.5, 129.1, 129.2, 130.1, 131.0, 131.3, 131.5, 133.6, 136.3, 138.8, 167.5.

### Example 19b: Preparation of Compound 11

To a stirred solution of compound 14 (100 mg, 0.29 mmol) in acetic acid (5 mL) was added lead dioxide (77.2 mg, 0.32 mmol) and bromine (0.02 mL, 0.32 mmol). Stirring was continued for 6 hrs at room temperature. The mixture was poured in a solution of Na₂S₂O₅ and was extracted with EtOAc (3×20 mL). The collected organic phases were washed with water and brine, and then they were dried over Na₂SO₄. Titration with a mixture of Et₂O/Acetone afforded compound 11 (40 mg, 0.09 mmol) as white solid. Yield 33%. ¹H NMR (400 MHz, DMSO) δ 4.44 (s, 2H), 7.42 (t, J=7.6 Hz, 1H), 7.48 (m, 3H), 7.61-7.65 (m, 3H), 7.82 (d, J=7.5 Hz, 1H), 8.0 (s, 1H), 13.1 (m, 2H). ¹³C NMR (100 MHz, DMSO) δ 33.9, 128.4, 128.6, 129.14, 129.3, 129.3, 129.3, 130.1, 130.2, 131.3, 133.9, 138.1, 138.4, 167.5. HPLC: 94.2%

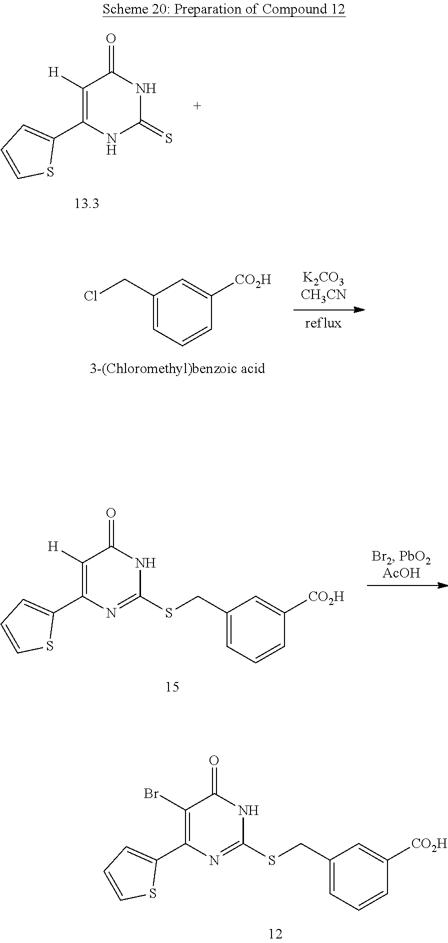

### Example 20a: Preparation of Compound 15

To a stirred suspension of intermediate 13.3 (235 mg, 1.11 mmol) and K₂CO₃ (460 mg, 3.33 mmol) in CH₃CN (15 mL) was added 3-(chloromethyl)benzoic acid (190 mg, 1.11 mmol). Stirring was continued overnight at reflux. The volatiles were removed under vacuo. The crude was taken up with water, washed with EtOAc, acidified to pH 3 and extracted with EtOAc (3×50 mL). Titration with a mixture of Et₂O/Acetone afforded compound 15 (150 mg, 0.44 mmol) as white solid. Yield 39%. ¹H NMR (400 MHz, DMSO) δ 4.55 (s, 2H), 6.64 (s, 1H), 7.19 (dd, J=4.9 Hz, J=3.8 Hz, 1H), 7.43 (t, J=7.7 Hz, 1H), 7.74 (d, J=7.6 Hz, 1H), 7.77 (d, J=4.9 Hz, 1H), 7.81 (d, J=7.8 Hz, 1H), 7.90 (d, J=3.6 Hz, 1H), 8.08 (s, 1H), 12.80 (s, 2H). ¹³C NMR (100 MHz, DMSO) δ 33.5, 101.6, 128.1, 128.6, 129.1, 129.1, 130.1, 131.1, 131.4, 133.7, 138.9, 141.7, 167.4.

### Example 20b: Preparation of Compound 12

To a stirred solution of compound 15 (134 mg, 0.39 mmol) in acetic acid (5 mL) was added lead dioxide (102 mg, 0.42 mmol) and bromine (0.022 mL, 0.42 mmol). Stirring was continued for 6 hrs at room temperature. The mixture was poured in a solution of Na₂S₂O₅ and was extracted with EtOAc (3×20 mL). The collected organic phases were washed with water and brine, and then they were dried over Na₂SO₄. The crude of reaction was subjected to flash chromatography purification eluting with CH₂Cl₂/MeOH (10% for product). Compound 12 (45 mg, 0.11 mmol) was obtained as white solid. Yield 27%. ¹H NMR (400 MHz, DMSO) δ 4.56 (s, 2H), 7.27 (t, J=3.5 Hz, 1H), 7.44 (t, J=7.6 Hz, 1H), 7.72 (d, J=7.5 Hz, 1H), 7.82 (d, J=7.7 Hz, 1H), 7.92 (d, J=4.5 Hz), 8.07 (s, 1H), 8.33 (d, J=2.9 Hz, 1H), 13.1 (m, 2H). ¹³C NMR (100 MHz, DMSO) δ 33.8, 128.6, 128.7, 128.7, 129.2, 130.1, 131.4, 132.3, 132.8, 133.6, 138.3, 141.1, 152.1, 158.5, 159.6, 167.4. HPLC:

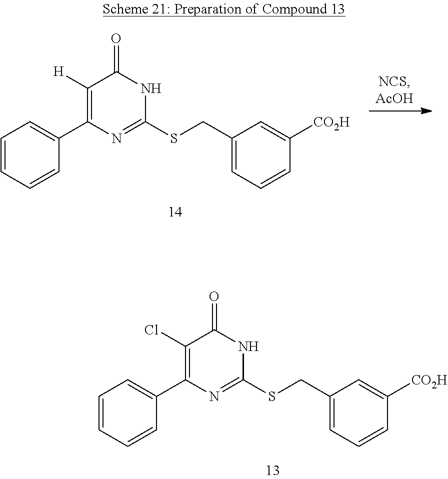

### Example 21: Preparation of Compound 13

To a stirred solution of compound 14 (100 mg, 0.29 mmol) in acetic acid (5 mL) was added lead dioxide (55.8 mg, 0.35 mmol) and N-chlorosuccinimide (47 mg, 0.35 mmol). Stirring was continued for 6 hrs at room temperature. The mixture was poured in water and was extracted with EtOAc (3×20 mL). The collected organic phases were washed with water and brine, and then they were dried over Na₂SO₄. Titration with a mixture of Et₂O/acetone afforded compound 13 (40 mg, 0.1 mmol) as white solid. Yield 37%. ¹H NMR (400 MHz, DMSO) δ 4.47 (s, 2H), 7.43 (t, J=7.7 Hz, 1H), 7.49 (m, 3H), 7.64 (d, J=7.2 Hz, 1H), 7.71 (m, 2H), 7.83 (d, J=7.5 Hz, 1H), 8.02 (s, 1H), 13.1 (s, 1H), 13.25 (s, 1H); ¹³C NMR (100 MHz, DMSO) δ 33.9, 128.4, 128.4, 128.6, 129.1, 129.4, 130.2, 131.3, 131.3, 133.8, 136.5, 138.3, 167.5. HPLC: 95.3%

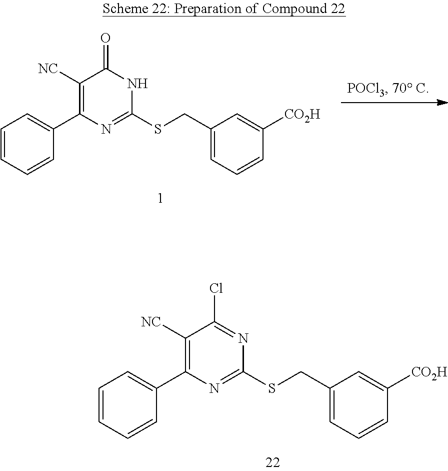 [SMILES: N#Cc1c(-c2ccccc2)nc(SCc2cccc(C(=O)O)c2)[nH]c1=O ; "POCl3, 70° C." ; N#Cc1c(Cl)nc(SCc2cccc(C(=O)O)c2)nc1-c1ccccc1]

### Example 22: Preparation of Compound 22

A stirred suspension of compound 1 (160 mg, 0.44 mmol) and POCl₃ (3 mL) was heated at 70° C. for 6 h. The white suspension turned red. The excess of POCl₃ was carefully destroyed with crushed ice and then water. The mixture was extracted with EtOAc (3×20 mL). The collected organic phase were washed with brine, dried over Na₂SO₄ and evaporated. Flash chromatography purification (gradient CH₂Cl₂/MeOH) afforded the title compound 22 (60 mg, 0.16 mmol) as white solid. Yield 36%. ¹H NMR (400 MHz, DMSO) δ 4.58 (s, 2H), 7.44 (t, J=7.7 Hz, 1H), 7.58-7.62 (m, 2H), 7.66 (d, J=7.17 Hz, 1H), 7.70 (d, J=7.7 Hz, 1H), 7.82 (d, J=7.7 Hz, 1H), 7.94 (d, J=7.1 Hz, 2H), 8.08 (s, 1H), 12.98 (s, 1H); ¹³C NMR (100 MHz, DMSO) δ 34.9, 102.5, 115.2, 128.7, 129.2, 129.2, 129.6, 129.6, 130.4, 131.4, 132.7, 133.9, 134.7, 138.0, 162.9, 167.5, 169.0, 174.3. HPLC: 98.8%

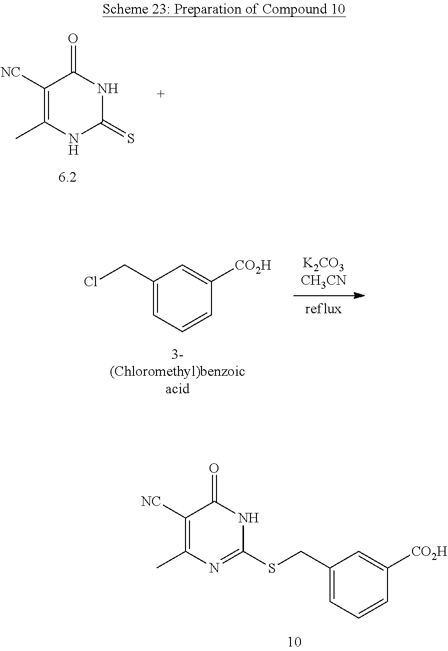 [SMILES: Cc1[nH]c(=S)[nH]c(=O)c1C#N ; O=C(O)c1cccc(CCl)c1 ; "K2CO3CH3CN" ; "reflux" ; Cc1nc(SCc2cccc(C(=O)O)c2)[nH]c(=O)c1C#N]

### Example 23: Preparation of Compound 10

To a stirred suspension of intermediate 6.2 (145 mg, 0.86 mmol) and K₂CO₃ (599 mg, 4.33 mmol) in CH₃CN (15 mL) was added 3-(chloromethyl)benzoic acid (148 mg, 0.86 mmol). Stirring was continued overnight at reflux. The volatiles were removed under vacuo. The crude was taken up with water, washed with EtOAc, acidified to pH 3 and extracted with EtOAc (3×50 mL). The crude of reaction was purified by flash chromatography, eluting with (CH₂Cl₂/MeOH+ACOH 3%) affording the title compound 10 (60 mg, 0.2 mmol) as white solid. Yield 23%. ¹H NMR (400 MHz, DMSO) δ 2.44 (s, 3H), 4.49 (s, 2H), 7.45 (t, J=7.6 Hz, 1H), 7.68 (d, J=7.4 Hz, 1H), 7.82 (d, J=7.6 Hz, 1H), 8.05 (m, 1H), 13.1 (s, 1H); ¹³C NMR (100 MHz, DMSO) δ 23.3, 33.9, 95.3, 115.6, 128.7, 129.1, 130.6, 131.2, 134.1, 138.0, 161.1, 165.7, 167.4, 170.9. HPLC 96.5%

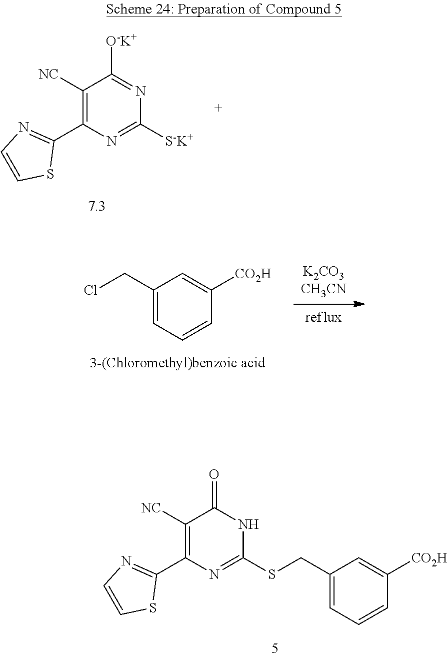

### Example 24: Preparation of Compound 5

To a stirred suspension of intermediate 7.3 (414 mg, 1.27 mmol) and K₂CO₃ (526 mg, 3.81 mmol) in CH₃CN (20 mL) was added 3-(chloromethyl)benzoic acid (217 mg, 1.27 mmol). Stirring was continued overnight at reflux. The volatiles were removed under vacuo. The crude was taken up with water, washed with EtOAc, acidified to pH 3 and extracted with EtOAc (3×50 mL). The title compound 5 has been obtained (260 mg, 0.7 mmol) as pure light yellow solid after titration with a mixture of Et₂O/Acetone. Yield 55%. ¹H NMR (400 MHz, DMSO) δ 4.62 (s, 2H), 7.45 (m, 1H), 7.74 (d, J=5.9 Hz, 1H), 7.82 (d, J=6.1 Hz, 1H), 8.08 (s, 1H), 8.21 (d, J=7.2 Hz, 2H), 12.9 (s, 1H); ¹³C NMR (100 MHz, DMSO) δ 34.0, 90.5, 114.9, 127.7, 128.8, 129.3, 130.1, 131.4, 133.7, 138.1, 146.2, 156.7, 161.9, 163.8, 166.5, 167.4. HPLC 96.5%

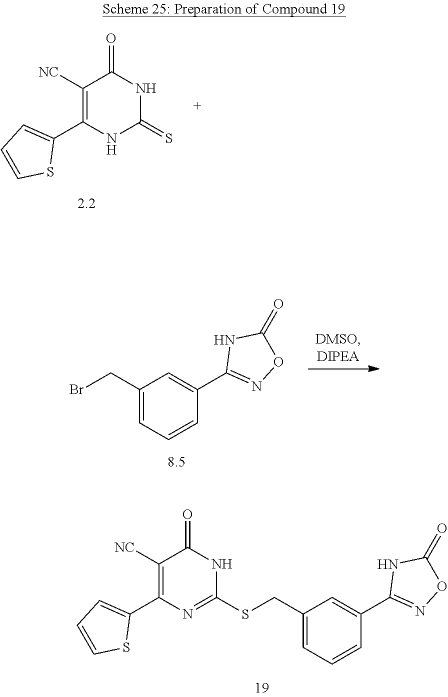 [SMILES: N#Cc1c(-c2cccs2)[nH]c(=S)[nH]c1=O ; O=c1[nH]c(-c2cccc(CBr)c2)no1 ; "DMSO,DIPEA" ; N#Cc1c(-c2cccs2)nc(SCc2cccc(-c3noc(=O)[nH]3)c2)[nH]c1=O]

### Example 25: Preparation of Compound 19

To a stirred suspension of intermediate 2.2 (100 mg, 0.42 mmol) and DIPEA (0.07 mL, 0.47 mmol) in DMSO (5 mL) was added intermediate 8.5 (120 mg, 0.47 mmol). Stirring was continued overnight at room temperature. The crude was poured in water, washed with EtOAc then acidified to pH 3 and extracted with EtOAc (3×50 mL). The title compound 19 has been obtained (65 mg, 0.15 mmol) as pure orange solid after flash chromatography purification eluting with CH₂Cl₂/MeOH (10% for product). Yield 38%. ¹H NMR (400 MHz, DMSO) δ 4.37 (s, 2H), 7.20 (t, J=4 Hz, 1H), 7.45 (t, J=7.6 Hz, 1H), 7.63 (t, J=7.2 Hz, 2H), 7.75 (d, J=4.3 Hz, 1H), 7.88 (s, 1H), 8.07 (d, J=3 Hz, 1H); ¹³C NMR (100 MHz, DMSO) δ 33.7, 85.7, 120.4, 124.9, 125.4, 126.7, 128.7, 128.8, 129.5, 131.1, 132.3, 140.6, 142.2, 159.1, 159.9, 163.3, 170.4, 171.3. HPLC 94.1%.

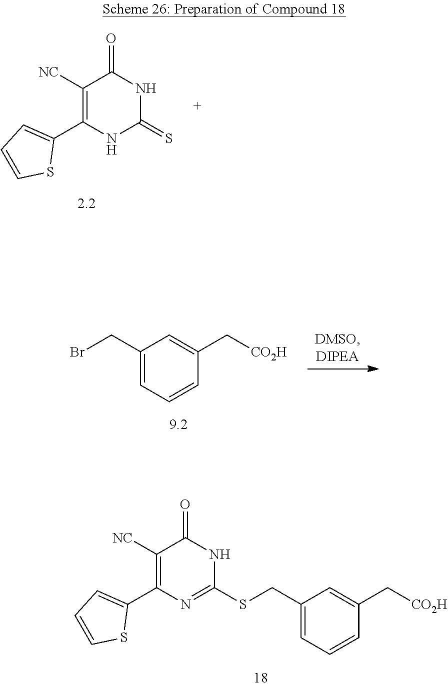 [SMILES: N#Cc1c(-c2cccs2)[nH]c(=S)[nH]c1=O ; O=C(O)Cc1cccc(CBr)c1 ; "DMSO,DIPEA" ; N#Cc1c(-c2cccs2)nc(SCc2cccc(CC(=O)O)c2)[nH]c1=O]

### Example 26: Preparation of Compound 18

To a stirred suspension of intermediate 2.2 (500 mg, 0. mmol) and DIPEA (0.4 mL, 2.12 mmol) in DMSO (5 mL) was added intermediate 9.2 (487 mg, 2.12 mmol). Stirring was continued overnight at room temperature. The crude was poured in water, washed with EtOAc then acidified to pH 3 and extracted with EtOAc (3×50 mL). The title compound 18 has been obtained (200 mg, 0.52 mmol) as pure yellowish solid after flash chromatography purification eluting with CH₂Cl₂/MeOH (10% for product) and titration with a mixture of Et₂O/Acetone. Yield 25%. ¹H NMR (400 MHz, DMSO) δ 3.49 (s, 2H), 4.53 (s, 2H), 7.16 (d, J=6.8 Hz, 1H), 7.26 (t, J=7.2 Hz, 1H), 7.36 (m, 3H), 8.05 (d, J=4.4 Hz, 1H), 8.27 (s, 1H), 12.13 (s, 1H); ¹³C NMR (100 MHz, DMSO) δ 34.3, 40.9, 88.5, 116.8, 127.6, 128.9, 129.1, 129.8, 130.4, 131.9, 135.2, 135.8, 137.0, 139.9, 159.1, 161.6, 165.7, 172.9. HPLC 95.8%.

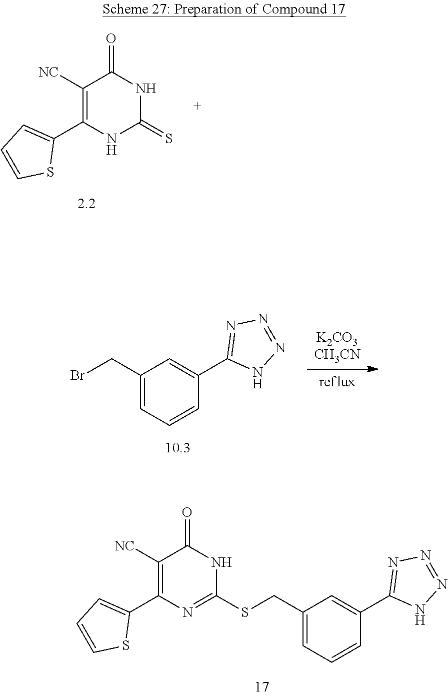 [SMILES: N#Cc1c(-c2cccs2)[nH]c(=S)[nH]c1=O ; BrCc1cccc(-c2nnn[nH]2)c1 ; "K2CO3CH3CN" ; "reflux" ; N#Cc1c(-c2cccs2)nc(SCc2cccc(-c3nnn[nH]3)c2)[nH]c1=O]

### Example 27: Preparation of Compound 17

To a stirred suspension of intermediate 2.2 (160 mg, 0.66 mmol) and DIPEA (0.09 mL, 0.55 mmol) in DMSO (3 mL) was added intermediate 10.3 (171 mg, 0.55 mmol). Stirring was continued overnight at room temperature. The crude was poured in water, washed with EtOAc then acidified to pH 3 and extracted with EtOAc (3×50 mL). The title compound 17 has been obtained (90 mg, 0.22 mmol) as pure orange solid after flash chromatography purification eluting with CH₂Cl₂/MeOH (5% for product) and prior titration with a mixture of Et₂O/Acetone. Yield 23%. ¹H NMR (400 MHz, DMSO) δ 4.59 (s, 2H), 7.29 (t, J=4.6 Hz, 1H), 7.54 (t, J=7.6 Hz, 1H), 7.68 (t, J=7.8 Hz, 1H), 7.91 (d, J=7.7 Hz, 1H), 7.96 (d, J=4.9 Hz, 1H), 8.16 (s, 1H), 8.22 (d, J=3.8 Hz, 1H); ¹³C NMR (100 MHz, DMSO) δ 33.9, 88.9, 117.6, 125.0, 126.2, 127.9, 129.6, 129.9, 131.2, 131.9, 134.4, 139.3, 140.4, 155.8, 159.1, 163.8, 167.0. HPLC 96.2%.

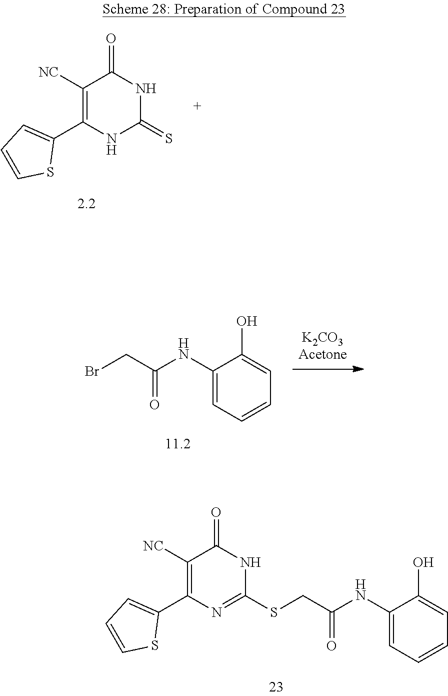 [SMILES: N#Cc1c(-c2cccs2)[nH]c(=S)[nH]c1=O ; O=C(CBr)Nc1ccccc1O ; "K2CO3Acetone" ; N#Cc1c(-c2cccs2)nc(SCC(=O)Nc2ccccc2O)[nH]c1=O]

### Example 28: Preparation of Compound 23

To a stirred suspension of intermediate 2.2 (150 mg, 0.63 mmol) and K₂CO₃ (96.6 mg, 0.70 mmol) in acetone (10 mL) was added intermediate 11.2 (173 mg, 0.72 mmol). Stirring was continued overnight at room temperature. The volatiles were removed under vacuo. The crude was taken up with water, acidified to pH 3 and extracted with EtOAc (3×50 mL). The title compound 23 has been obtained (150 mg, 0.39 mmol) as pure orange solid after flash chromatography purification eluting with CH₂Cl₂/MeOH (5% for product) and prior titration with hot EtOAc. Yield 62%. ¹H NMR (400 MHz, DMSO) δ 3.92 (s, 2H), 6.72 (t, J=7.7 Hz, 1H), 6.79 (d, J=7.6 Hz, 1H), 6.88 (t, J=7.3 Hz, 1H), 7.20 (t, J=4.26 Hz, 1H), 7.77 (d, J=4.7 Hz, 1H); 7.92 (d, J=7.8 Hz, 1H), 8.0 (d, J=3.5 Hz, 1H); 9.59 (s, 1H), 9.80 (s, 1H); ¹³C NMR (100 MHz, DMSO) δ 35.12, 86.2, 115.5, 119.2, 120.1, 121.0, 124.4, 127.0, 128.7, 129.2, 131.4, 141.8, 147.3, 159.2, 167.9, 169.1, 170.5. HPLC 97.7%.

### Example 29: Synthesis of Exemplified Compounds

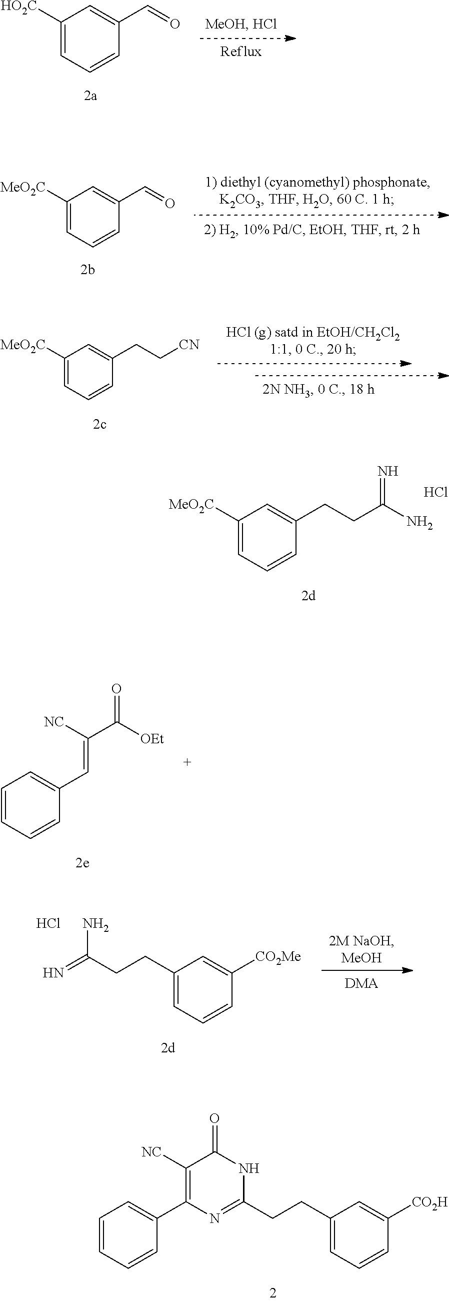

See, Hirose M, et al., “Design and synthesis of novel DFG-out RAF/vascular endothelial growth factor receptor 2 (VEGFR2) inhibitors: 3. Evaluation of 5-amino-linked thiazolo[5,4-d]pyrimidine and thiazolo[5,4-b]pyridine derivatives.” Bioorg. Med. Chem. 2012, 15; 20(18):5600-15.

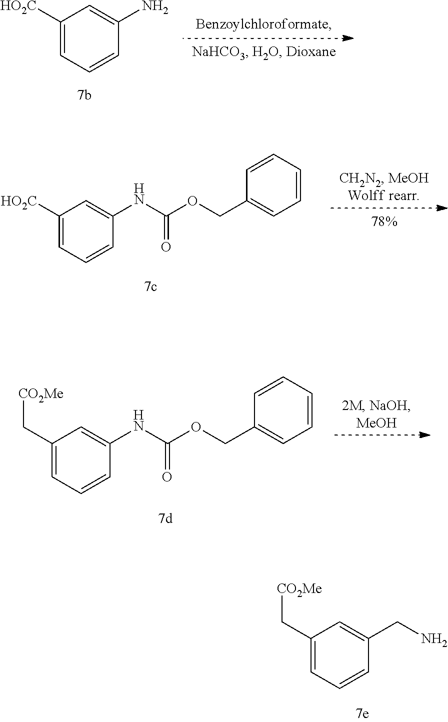

See, Clift M D, Silverman R B., “Synthesis and evaluation of novel aromatic substrates and competitive inhibitors of GABA aminotransferase,” Bioorg. Med. Chem. Lett,. 2008, 15; 18(10):3122-5.

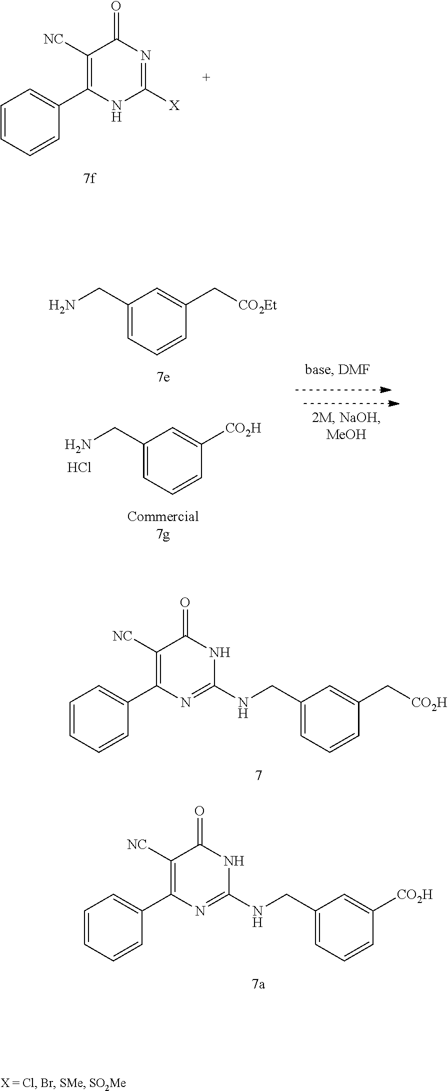

A. M. El-Reedy, A. O. Ayyad and A. S. Ali, “Azolopyrimidines and pyrimidoquinazolines from 4-chloropyrimidines,” J. Het. Chem. 1989, 26, 313-16.

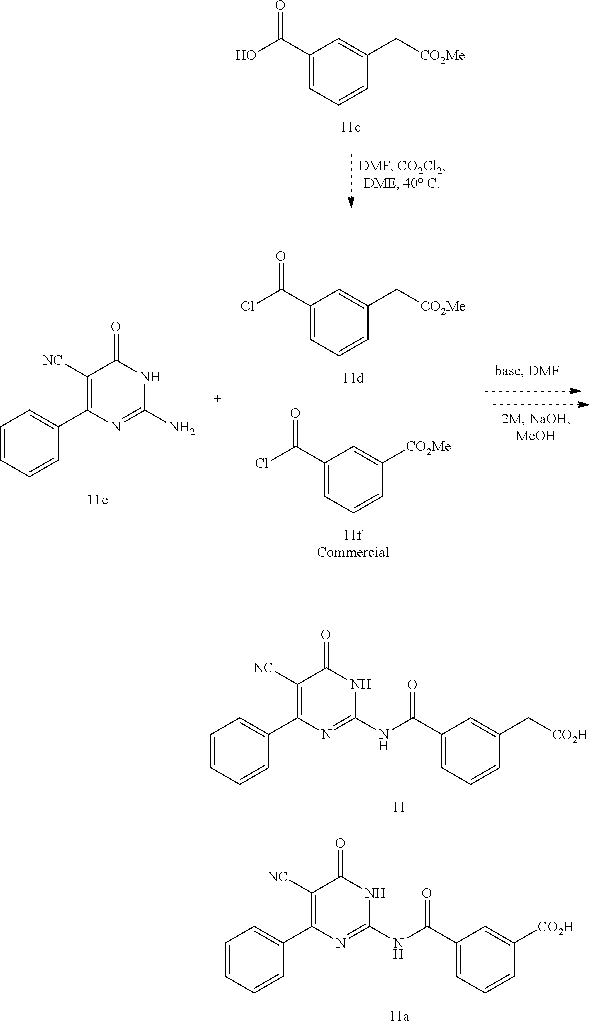

See, Iwahashi M, et al., “Design and synthesis of new prostaglandin D₂ receptor antagonists,” Bioorg. Med. Chem. 2011, 19(18):5361-71.

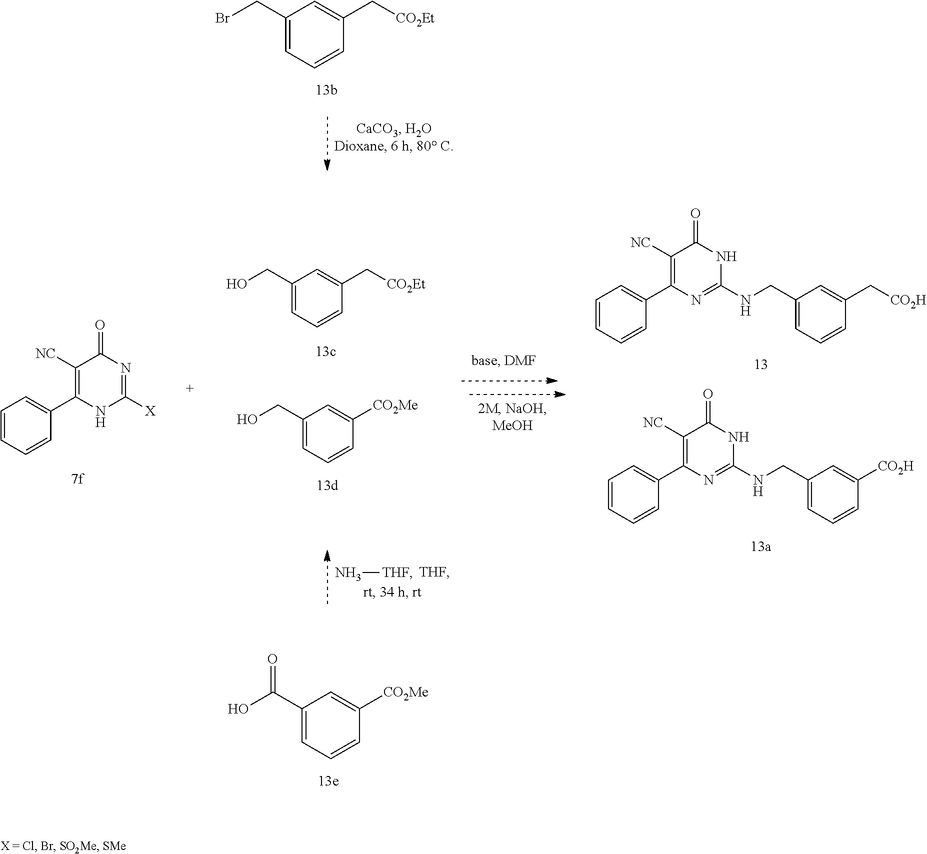

See, U.S. 2008/004,302(A1); and U.S. Pat. No. 8,716,470 (B2).

### Biological Activity

### ACMSD and Acute Inflammation

### Cellular Assays

Assay Protocol for Mouse Kupffer cell Transfection with ACMSD Plasmid and Cytokine Analysis after LPS Induction

Kupffer cells (immortalized mouse Kupffer cell line, (cat #SCC119 (ImKC) Merck Millipore) were plated out in a tissue culture 24 well plate with 150,000 cells/well in RPMI medium+10% FBS. At 24 h after plating, the cells were transfected with Fugene HD (Promega), pCDNA3.1 mouse ACMSD, and pCDNA3.1 empty vectors each at the concentration of 0.75 μg/well for 18 hrs.

Cell stimulation with the ACMSD inhibitor (e.g. compounds of present disclosure, compound of Formula I, Ia, Ib, and II) was performed using the following concentrations of test compound, 0.5, 5, and 50 μM, with cells where only DMSO at the final concentration of 0.5% was added used as control. All the wells were normalized with DMSO at 0.5% final concentration. Cells were treated with final concentration of 50 ng/ml of LPS in cell medium for 18 hrs, the supernatants were then collected for analysis of mouse cytokine secretion (IL-1α, IL1β, IL-2, IL-3, IL-4, IL-5, IL6, IL-9, IL-10, IL-12 (p40), IL-12 (p70), IL-13, IL-17, IFN-γ, Rantes, Eotaxin, MCP-1, MIP-1α, MIP-1β, G-CSF, GM-CSF, TNFα, and KC (Keratinocyte chemoattractant)) using the Bio-Plex Pro mouse cytokine 23-plex assay (cat #M60009RDPD). The cells were centrifuged and the resulting pellets were collected for either RT PCR analysis or ATP measure (Promega Cell-Titer-Glo cat #G7571).

### Assay Protocol for ACMSD Silencing

Kupffer cells plated out in a 24 well plate were transfected with 5 pmol of either ACMSD siRNA or scrambled siRNA as a negative control (siRNA ACMSD cat #4390771 and scrambled siRNA cat #4390843, both Ambion). After 18 hrs of incubation the cells were treated with the ACMSD inhibitor (e.g. compounds of present disclosure, compound of Formula I, Ia, Ib, and II) (5 μM) or DMSO as vehicle, and then after an additional 1 hr with final concentration of 50 ng/ml of LPS in cell medium, followed by overnight incubation before cytokine secretion was then measured using the Bio-Plex kit. All the wells were normalized with DMSO at 0.5% final concentration.

### Cytokine Secretion Measurement

200,000 Kupffer cells were plated out in a 24 well plate and the ACMSD inhibitor (e.g. compounds of present disclosure, compound of Formula I, Ia, Ib, and II) (5 μM) was added 1 hr before the cells were stimulated with LPS overnight 50 ng/ml (1 mg/mL stock solution in H₂O, dispensed at 50 ng/mL in the well) final concentration. Cells treated with DMSO were used as control at the final concentration of 0.5% in medium. All the wells were normalized with DMSO at 0.5% final concentration. The supernatant was collected to measure cytokine secretion using a Bio-Plex Pro mouse cytokine 23-plex assay system (cat #M60009RDPD) using a Bio-Plex Instrument. The cells were washed once with PBS, and then lysed to extract total RNA for performing RT-PCR to evaluate ACMSD modulation of IL-10, SIRT1, and STAT3 gene expression.

Briefly, total RNA was extracted from the cells using the manufacturer's protocol for Qiagen RNeasy Plus Mini Kit (cat. 74134). The total RNA concentration was quantified using an Implen Instrument, and the purity was evaluated by measuring the ratio of A260/A280. Isolated RNA had an A260/A280 ratio of (1.8-2.0 range).

Total RNA (1 μg) was reverse transcribed using the manufacturer's protocol for SuperScript IV VILO Master Mix (ThermoFisher cat. #. 11756500). Real-time PCR was then performed in CFX 96 Real Time System (Bio-Rad) as follows: denaturation at 95° C. for 2 minutes, followed by 40 cycles at 95° C. for 10 s, and hybridization at 60° C. for 20 s. QPCR was performed using the 2× QuantiNova SYBR Green PCR Master Mix (Qiagen). Mouse β2 microglobulin (m 02M) was used as a reference gene.

All cytokine secretion values were normalized to the luminescent signal relative to the measuring of the cellular ATP (CellTiter-Glo cat #G7571)).

Primer Sequences
mSTAT3_FW
CACATGCCACGTTGGTGTTT
mSTAT3_RW
ACGATCCGGGCAATTTCCAT
mIL10_FW
CAGTACAGCCGGGAAGACAAT
mIL10_RW
TTGGCAACCCAAGTAACCCT
mSIRT1_FW
TATCTATGCTCGCCTTGCGG
mSIRT1_RW
GACACAGAGACGGCTGGAAC
mACMSD_FW
GCAGATGGATGGACGAATGG
mACMSD_RW
CGAAGCACACTTTGAGTTTGG
mB2M_FW
CTCGGTGACCCTGGTCTTTC
mB2M_RW
GGATTTCAATGTGAGGCGGG

### Animal Model Protocols

### LPS-induced Acute Kidney and Liver Injury

A total of 40 Sprague-Dawley rats (8 week old male rats, 200-220 g) were randomly divided into 3 LPS model groups (8 h, 24 h, and 48 h sacrifice groups n=10 in each group) and the control group (n=10 per group). The rats were obtained from Charles River Laboratories. After acclimatization for 1 week, the rats in the LPS groups were intraperitoneally injected with 10 mg/kg LPS (dissolved in normal saline) which was based on earlier reports and the rats in the control group were intraperitoneally injected with an equivalent amount of normal saline. After 8 h, 24 h, or 48 h the rats were anesthetized using chloral hydrate, and then blood was collected by direct puncture of the abdominal aorta. The blood was then centrifuged at 3000×g to prepare a serum sample. Organ tissues were collected and divided into two parts; one part was fixed in 4% formaldehyde, and the other was frozen in liquid N₂. All serum and tissue samples were stored at −80° C. until biochemical analysis for liver function (AST/ALT levels), kidney function (BUN and serum/plasma creatinine levels), and inflammatory biomarkers (cytokine and chemokines expression and secreation, including; TNFα, IL-6, MCP-1. MIP-1α, and IL-10).

### CLP-induced Sepsis Model

C57BL/6 mice (12-15 weeks old) (obtained from Charles River Laboratories) were anesthetized by intraperitoneal (i.p.) injection of a 1:1 solution of ketamine (75 mg/kg) and xylazine (15 mg/kg) (or pentobarbital (70 mg/kg)). The lower abdomen of the mouse was shaved and the area disinfected with a 70% alcohol swab. Under aseptic conditions, a 1 to 2 cm midline laparotomy was performed and the cecum exposed. The cecum was tightly ligated at the level of the second cecal artery with a 2.0 silk suture, perforated twice with a 22-gauge needle, and after expressing to allow for fecal matter to extrude, returned into the abdomen. The abdominal wall is then closed by planes using a running silk 4-0. Control animals underwent the same laparotomy and externalization of the cecum, but no ligation or perforation was performed. Animals were resuscitated with 1.0 mL of 0.9% normal saline, immediately after surgery via subcutaneous injection and recovered on thermal blankets under monitoring. Animals were then treated with 0.5 ml LRS (Q12 hours SQ for 3 days), ampicillin sulbactam (250 mg/kg Q12 hours IP for 3 days) and analgesic treatment (buprenorphine 0.05 mg/kg for 3 days).

ACMSD inhibitors (e.g. compounds of present disclosure, compound of Formula I, Ia, Ib, and II) were administered at two different timepoints: (a) an early phase timepoint i.e. at the time of CLP and (b) at 24 hours after CLP. A dose of 15 mg/kg IP injection of the ACMSD inhibitor was used. Blood samples and target organ tissue (liver and kidney) were collected at the time of sacrificing for histology and biomarker measurements.

### LPS-induced Sepsis Model

Male C57/6 mice (12-15 weeks old) (obtained from Charles River Laboratories) were housed under a 12 h/12 h light/dark cycle with free access to water and standard food. The mice were divided into four groups: control, control+ACMSD inhibitor, LPS, and LPS+the ACMSD inhibitor. LPS (Sigma; 20 mg/kg/d and 1 mg/ml in 0.9% normal saline) was intraperitoneally injected, and the same volume of 0.9% normal saline alone was used as controls. A dose of 15 mg/kg IP injection of the ACMSD inhibitor (e.g. compounds of present disclosure, compound of Formula I, Ia, Ib, and II) was used which was intraperitoneally injected immediately after LPS injection. An equal volume of the solvent was injected in the control and LPS alone groups. The mice were sacrificed at 24 h after various treatments and the liver and kidneys were collected for subsequent analysis.

Blood samples and target organ tissue (liver and kidney) were collected at the time of sacrificing for histology and biomarker measurements.

### Acute Pancreatitis Model

Male C57BL/6 mice (8 week old) (obtained from Charles River Laboratories) were fed a standard commercial diet while housed at an ambient temperature of 20-22° C. with a relative humidity of 50±5% under a 12/12 h light/dark cycle in a pathogen-free facility. Experiments were performed on mice weighing between 20 and 25 g, and all mice were age matched to within 3 days.

Mice were fasted for 17 h before treatment but provided with free access to water. Acute pancreatitis was induced by six injections of caerulein (50 μg/kg, intraperitoneal [i.p.] at intervals of 1 h) as described previously. Each experimental group was composed of five mice. The control group received an i.p. injection of saline (0.9% NaCl) solution. In the combined caerulein and ACMSD inhibitor group, the ACMSD inhibitor (e.g. compounds of present disclosure, compound of Formula I, Ia, Ib, and II) (15 mg/kg bodyweight) was dissolved in vehicle (corn oil) were orally injected at 3 h before the first caerulein injection. All mice were sacrificed at 6 h after the last caerulein injection. Blood samples were taken to determine the serum amylase, lipase, and cytokine levels. A portion of the pancreas was fixed overnight in 4% paraformaldehyde in phosphate-buffered saline (PBS, pH 7.4) at 4° C. for immunohistochemical studies, embedded in paraffin, cut into 4-μm thick sections, which were then stained with hematoxylin and eosin (H&E) to observe the morphological changes under a light microscope by standard procedures. After staining with H&E, histological injury score of pancreatic slides were graded in a blinded manner, without knowledge of the experimental design, according to the severity and extent of edema, inflammatory cell infiltration, and acinar necrosis. A portion of the pancreas was also frozen in liquid nitrogen for western blotting and RT-PCR analysis.

### Acute Hepatic Injury Model

Male Balb/c mice (6-8 weeks old, 20±2 g) (obtained from Charles River Laboratories), were housed in plastic cages with controlled light and dark cycles and fed a standard diet with water in a controlled temperature (25±1° C.) and humidity (50±5%) environment. Hepatic injury was invoked with an injection of Concanavalin-A (20 mg/kg body weight) into the tail vein. The mice were randomly divided into six groups of ten mice as follows: (1) saline control group, (2) ACMSD inhibitor-alone group, (3) Concanavalin-A induced model group, (4) low-dose ACMSD inhibitor+Concanavalin-A group, (5) medium-dose ACMSD inhibitor+Concanavalin-A group, and (6) high-dose ACMSD inhibitor+Concanavalin-A group. For the first two weeks, the mice in the ACMSD inhibitor (e.g. compounds of present disclosure, compound of Formula I, Ia, Ib, and II) group received 15 mg/kg bodyweight/day by oral administration. The remaining mice received a 0.5% carboxymethyl cellulose solution at 0.1 mL/10 g bodyweight/day. At the fourteenth day, one hour after oral administration, Concanavalin-A (0.05 mL/10 g) was injected into the caudal veins of mice, except for the saline and ACMSD inhibitor-alone groups, which received only saline (0.05 mL/10 g) and after 8 hours, animals were sacrificed. The left hepatic lobes were stored at −80° C. until the IL-2, IL-6, and TNF-α assays were performed. The right hepatic lobes were fixed in 4% paraformaldehyde at 4° C. for hematoxylin-eosin (HE) and immunohistochemical staining.

Acute Graft vs Host Disease, aGvHD, (MHC mismatch) Model

Nine-week-old C57BL/6 (B6) (H-2 kb) and B/c (H-2 kd) mice were purchased from Charles River Laboratories. Mice were maintained under specific pathogen-free conditions in an animal facility at 22+1° C., 55+5% humidity, and a light/dark cycle of 12 h/12 h. The air in the facility was passed through a high-efficiency particulate air (HEPA) filter system to exclude bacteria and viruses. Animals were fed mouse chow and tap water ad libitum. Splenocytes (5×10⁶) and bone marrow cells (5×10⁶) were isolated from B6 donor mice and transplanted into B/c recipient mice via intra-venous (i.v.) injection. Before transplantation, B/c mice were sub-lethally irradiated at 690 cGy and allowed to rest for 2 hours. Following the induction of GvHD, the ACMSD inhibitor (e.g. compounds of present disclosure, compound of Formula I, Ia, Ib, and II) (15 mg/kg bodyweight/day per mouse) was administered orally to recipient mice daily, commencing on day 0 after bone marrow transplantation. Control GvHD mice received vehicle (saline), administered in the same manner as the treatment group. 10 mice were used in each group.

Survival after bone marrow transplantation was monitored daily and the extent of GvHD was assessed weekly using a scoring system that summed changes in the following clinical parameters: weight loss, posture, activity, fur texture, and skin integrity. Mice that were irradiated at 690 cGy were euthanized 28 days after bone marrow transplantation prior to blinded histopathology of GvHD target tissues.

Cytokine levels were measured from blood that was collected from the heart via cardiac puncture.

Histopathological and immunohistochemical analyses were performed on formalin-fixed skin, liver, and large and small intestinal tissue sections stained with hematoxylin and eosin. Epithelial loss, crypt damage, goblet cell depletion, and inflammatory cell infiltration were histologically scored.

siACMSD Cytokine Data

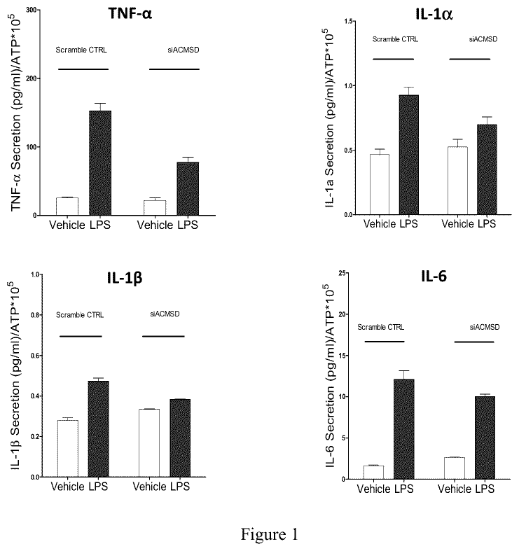

In Kupffer cells, silencing of ACMSD modulates secretion of both pro-inflammatory cytokines. Thus secretion of the pro-inflammatory cytokines; tumour necrosis factor α, TNFα, interleukins, IL-1α, IL-1β, and IL-6 in macrophages stimulated with LPS is reduced by short hairpin RNA, siACMSD, with respect to vehicle controls (FIG. 1).

### Inflammation and the Kynurenine Pathway (ACMSD & QPRT)

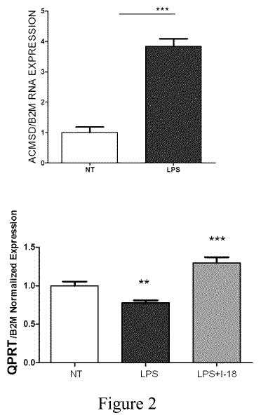

In Kupffer cells under inflammatory conditions (LPS at 50 ng/mL) the expression ACMSD is increased, whilst QPRT expression is reduced. Importantly ACMSD inhibition with compound I-18 increases QPRT expression beyond basal conditions (FIG. 2).

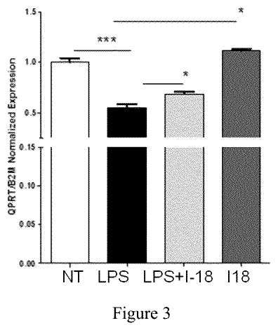

Similar results (QPRT down and up regulation) have been obtained in human primary bone marrow mononuclear cells (BMMCs) (FIG. 3).

Under inflammatory conditions the reduced expression of QPRT thus limits the biosynthesis of NAD⁺ from tryptophan via the de novo pathway. In contrast, treatment with an ACMSD inhibitor up regulates QPRT expression, thus restoring NAD⁺ biosynthesis.

### Restoration of NAD⁺ Biosynthesis

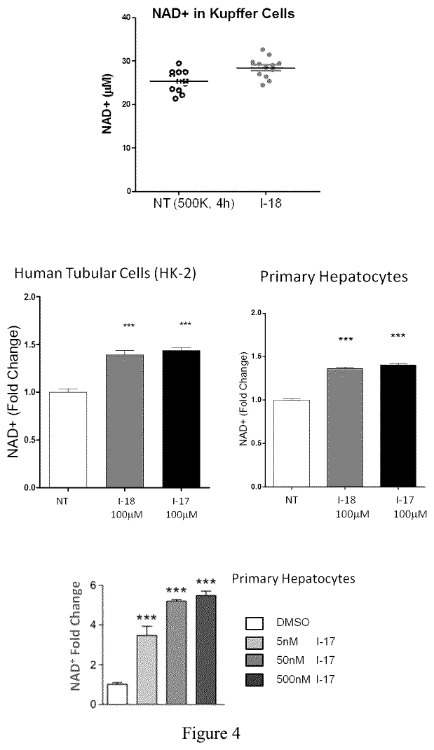

Inhibition of ACMSD with compound I-17 and compound I-18, restores NAD⁺ biosynthesis in Kupffer cells, primary hepatocytes, and kidney proximal tubular HK2 cells (FIG. 4).

### Effect of ACMSD Inhibition on Downstream Targets (SIRT1 & SIRT3)

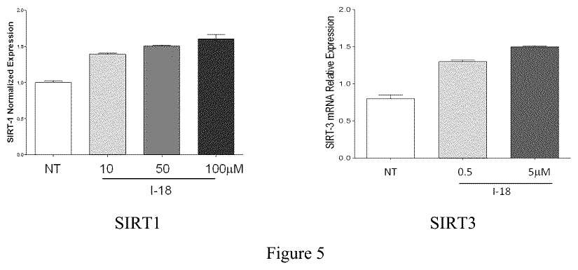

The NAD⁺ dependent SIRT1 is a key downstream target gene of NAD⁺ performs a wide variety of functions in biological systems and is an important regulator of energy homeostasis. SIRT1 also plays an important role in DNA damage repair and in maintaining genome integrity. SIRT1 is an important mediator between environmental stress and immune system activation and provides protection against inflammation by altering histones and transcription factors, such as NFκB and AP1. ACMSD inhibition with compound I-18 increases expression of both of the downstream target genes sirtuin, SIRT-1 in Kupffer cells and SIRT-3 in HK2 primary tubule cells (FIG. 5).

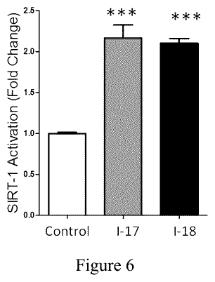

Both compounds I-17 and I-18 (at 500 nM) activate SIRT1 in primary hepatocytes measured at 24 hrs after treatment (FIG. 6).

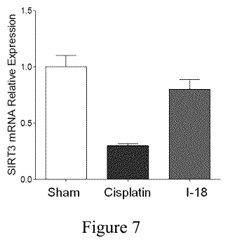

Inhibition of ACMSD with compound I-18 (50 μM) also reverses the reduction of SIRT expression induced by a Cisplatin insult in HK2 primary tubule cells (FIG. 7).

### Connection Between ACMSD Inhibition, SIRT, and the Inflammatory Response

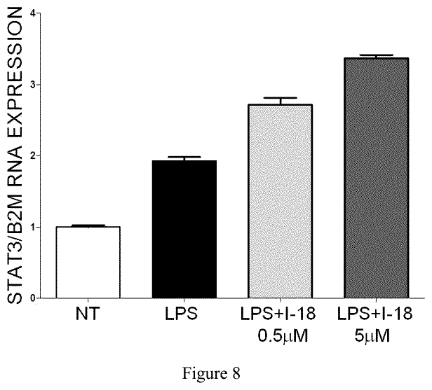

SIRT1 is a central component of the SIRT1/STAT3 pathway whereby increased expression and activity of SIRT1, induced by ACMSD inhibition with compound I-18, increases expression of signal transducer and activator of transport 3, STAT3, in Kupffer cells, in a dose-dependent manner (FIG. 8).

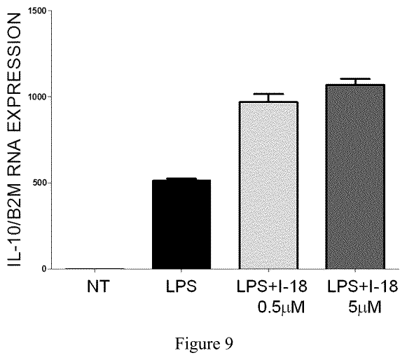

The Il-10/JAK1/STAT3 anti-inflammatory response is an essential negative regulator that controls both the degree and duration of inflammation. One of the main biological functions of IL-10 is to counter the production of inflammatory mediators, especially in response to TLR signalling. ACMSD inhibition with compound I-18 increases expression of IL-10, in Kupffer cells, in a dose-dependent manner to thus promote an anti-inflammatory response (FIG. 9).

STAT3 interacts with both IL-10 and Il-6. The binding of IL-10 to the IL-IOR results in the activation of JAK1 which induces STAT3 phosphorylation and STAT3 is a key effector molecule of IL-10 action. STAT3 activation is necessary for the IL-10-regulated anti-inflammatory effects.

Both IL-10 and interleukin-6 (IL-6) induce the activation of STAT3, but generate different cellular responses. IL-6 stimulation promotes a pro-inflammatory response, whilst IL-10 signalling induces a strong anti-inflammatory response.

### ACMSD Inhibition and Modulation of Cytokine Expression and Secretion

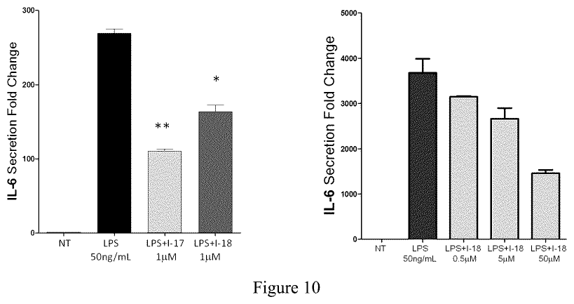

The secretion of the pro-inflammatory cytokine, IL-6 is reduced by inhibition of ACMSD with compounds I-17 and I-18 (1 μM), and in a dose-dependent manner with compound I-18 in Kupffer cells following LPS-induced inflammation. (Kupffer cells treated with LPS 50 ng/mL 16-18° C. incubation time 24 h both graphs) (FIG. 10).

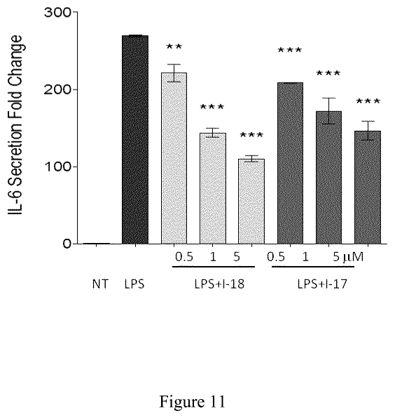

The secretion of the pro-inflammatory cytokine, IL-6 is also reduced in a dose-dependent manner by inhibition of ACMSD with compounds I-17 and I-18 in a co-culture primary hepatocytes and Kupffer cells following LPS-induced inflammation (50 ng/mL) (FIG. 11).

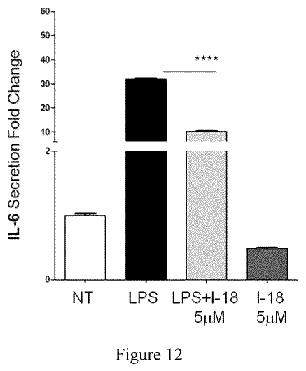

Inhibition of ACMSD with compound I-18 (5 μM), also reduces the secretion of LPS-induced IL-6 in human primary bone marrow mononuclear cells (BMMCs) (FIG. 12).

The concomitant reduction of IL-6 secretion and increase of IL-10 expression induced by ACMSD inhibitors thus provides a synergistic anti-inflammatory effect.

Beyond the reduction of IL-6 secretion and the increase of IL-10 expression, the inhibition of ACMSD has a broad anti-inflammatory effect via modulation of the expression and secretion of a range of both pro- and anti-inflammatory cytokines, chemokines, and other mediators of the acute inflammatory response across a variety of inflammatory cell types, as summarised below.

Thus, inhibition of ACMSD, with compounds I-17 and I-18 reduces the secretion and expression the following pro-inflammatory cytokines, TNFα, IL-1P, and IL-6 and the expression of the chemokine MCP-1.

### TNFα Secretion

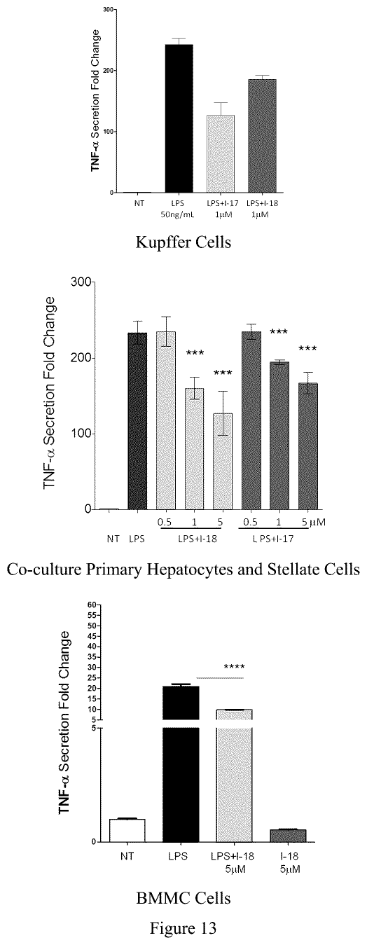

Inhibition of ACMSD, with compounds I-17 and I-18 in Kupffer cells, BMMC cells, and in a co-culture of primary hepatocytes and stellate cells, reduces the secretion of the inflammatory cytokine TNFα following LPS-induced inflammation (50 ng/mL) (FIG. 13).

### TNFα Expression

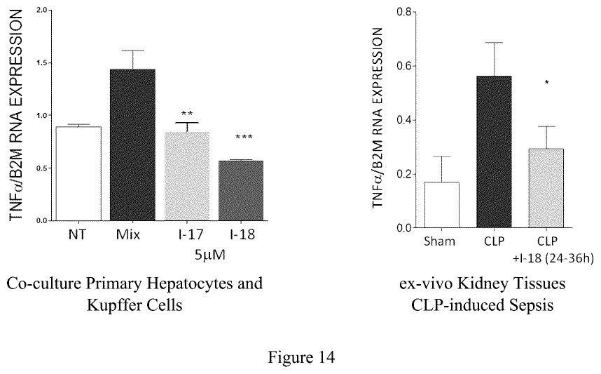

The expression of TNFα is reduced by inhibition of ACMSD with both compounds I-17 and I-18 (5 μM) in a co-culture of primary hepatocytes and stellate cells following treatment with a free fatty acid mix (after 6 hr exposure to a mixture of palmitate (0.33 mM) and oleate (0.66 mM)) and in ex-vivo kidney tissue samples from a CLP-induced sepsis model in mice following an IP 30 mg/kg/dose of compound I-18 (FIG. 14).

### IL-1β Secretion

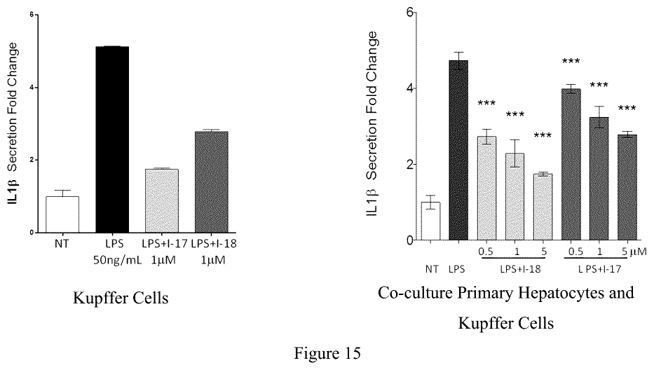

Inhibition of ACMSD, with compounds I-17 and I-18 (1 μM) in Kupffer cells and in a co-culture of primary hepatocytes and stellate cells, reduces the secretion of the inflammatory cytokine IL-1β following LPS-induced inflammation (50 ng/mL) (FIG. 15).

### IL-1β Expression

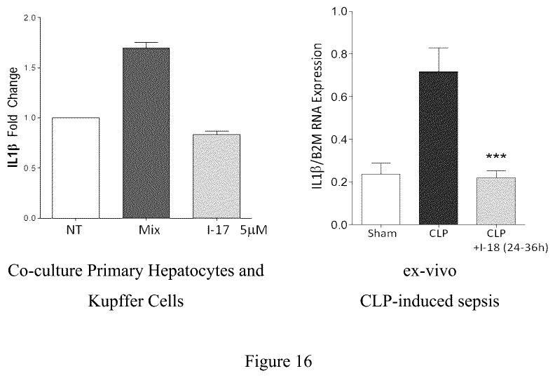

The expression of IL-1β is reduced by inhibition of ACMSD with compound I-17 (5 μM) in a co-culture of primary hepatocytes and stellate cells following treatment with a free fatty acid mix (after 6 hr exposure to a mixture of palmitate (0.33 mM) and oleate (0.66 mM)) and in ex-vivo kidney tissue samples from a CLP-induced sepsis model in mice, following an IP 30 mg/kg/dose of compound I-18 (FIG. 16).

### MCP1 Expression

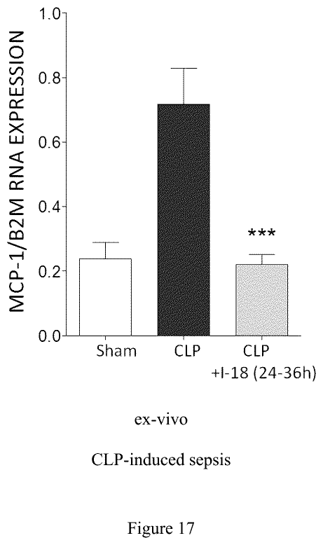

The expression of MCP1 is reduced by inhibition of ACMSD in ex-vivo kidney tissue samples from a CLP-induced sepsis model in mice, following an IP 30 mg/kg/dose of compound I-18 (FIG. 17).

### Anti-fibrotic/Anti-apoptotic Effects of ACMSD Inhibition

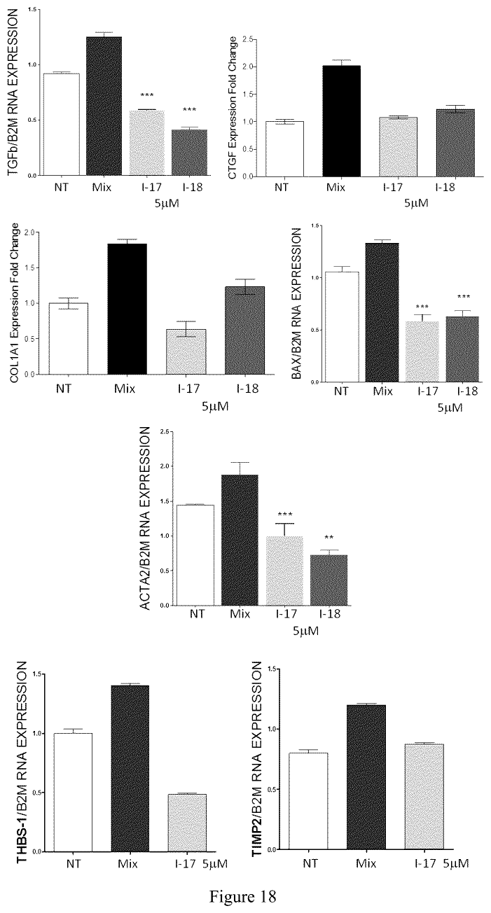

Inhibition of ACMSD with compound I-17 or compound I-18 (5 μM), reduces the expression of pro-fibrotic cytokine, TGF-β, the pro-fibrotic chemokines, CTGF, and the pro-fibrotic genes, Bcl-2-associated gene X, BAX, actin alpha 2, ACTA2, collagen type 1 alpha 1 chain, Col1A1, fibronectin, thrombospondin-1, THBS—1, and tissue inhibitor of metalloprotinases 2, TIMP2, in a co-culture of primary hepatocytes and stellate cells following treatment with a free fatty acid mix (after 6 hr exposure to a mixture of palmitate (0.33 mM) and oleate (0.66 mM)) (FIG. 18).

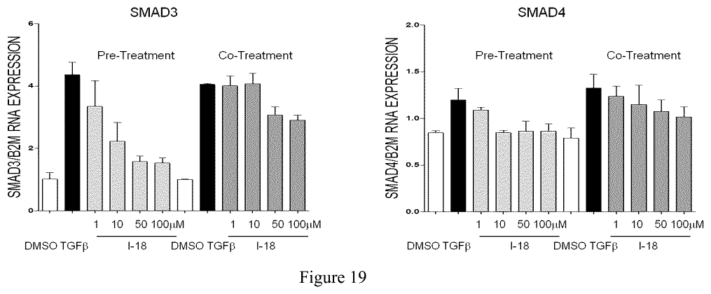

The anti-fibrotic effect of ACMSD inhibition via the reduction of transforming growth factor beta, TGF-β is mediated by the TGFβ/SMAD pathway as also shown by the dose-dependent reduction of mothers against decapentaplegic homologue 3, SMAD3, in both preventive and therapeutic treatment with compound I-18 following TGF-β-induced fibrosis in HK2 cells. SMAD4 expression was less modulated (FIG. 19).

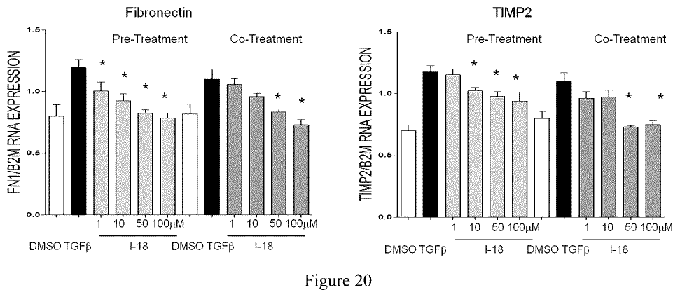

The anti-fibrotic effect of ACMSD inhibition is further demonstrated by a dose-dependent reduction in the expression of fibronectin and TIMP2 by compound I-18 (FIG. 20).

### ACMSD Inhibition and Mitochondria in the Inflammatory Setting

Mitochondria in Liver and Kidney Inflammatory Diseases

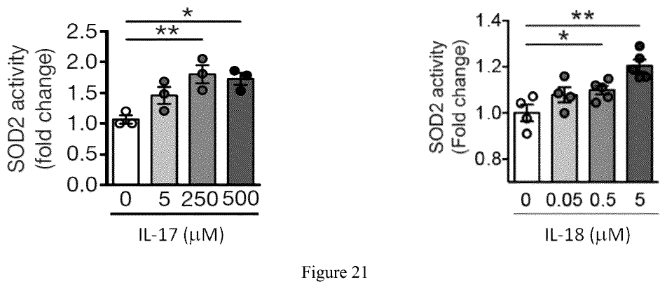

In mouse primary hepatocytes, the inhibition of ACMSD with either compound I-17 or I-18, increases the activity of mitochondrial superoxide dismutase 2, SOD2, in a dose-dependent manner, after treatment for 24 hrs (FIG. 21).

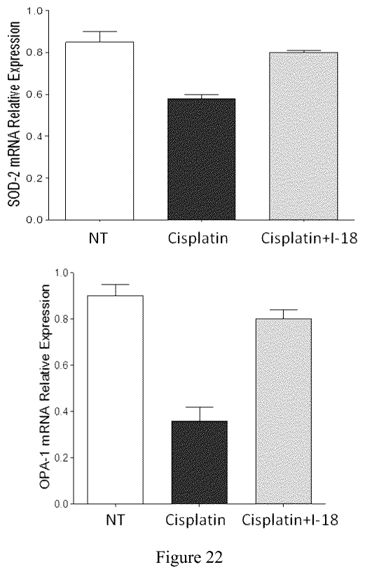

In HK-2 cells ACMSD inhibition with compound I-18 (100μM) also increases the expression of both SOD2 and mitochondrial dynamin-like 120 kDa protein, OPA-1, following cisplatin-insult (FIG. 22).

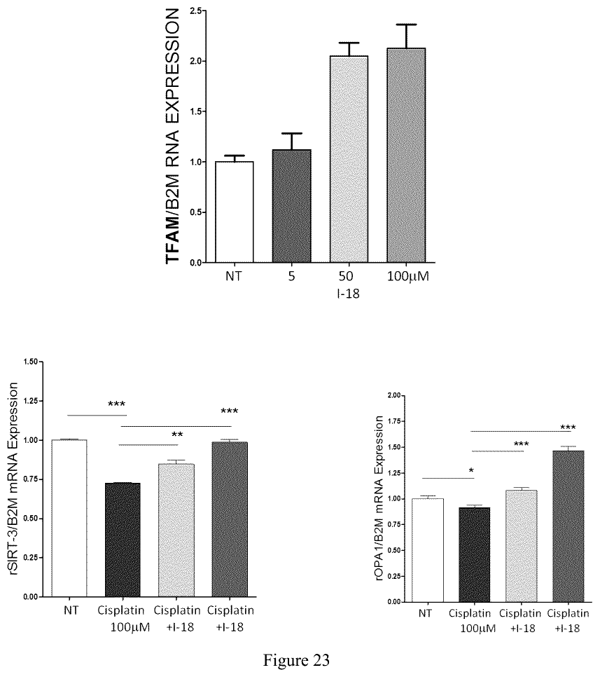

ACMSD inhibition with compounds I-17, (in various cell types) modulates cellular ROS production and mitochondrial biogenesis by increasing the expression of mitochondrial transcription factor A, TFAM in primary hepatocytes, via SIRT-1 activation, and OPA-1 a mitochondrial fusion protein in rat renal NRK52E cells, via SIRT-3 activation following Cisplatin insult (100 μM) (FIG. 23).

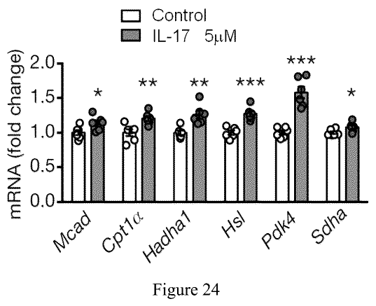

In primary hepatocytes inhibition of ACMSD with compound I-17 after 6 hr exposure to a mixture of palmitate (0.33 mM) and oleate (0.66 mM) increases the mRNA levels of fatty acid oxidation genes; medium-chain acyl-CoA dehydrogenase, Mcad, carnitine palmitoyltransferase 1, Cpt1a, hydroxyacyl-CoA dehydroenase, Hadha1, hormone sensitive lipase, Hsl, pyruvate dehydrogenase lipoamide kinase 4, Pdk4, and succinate dehydrogenase, Sdha (FIG. 24).

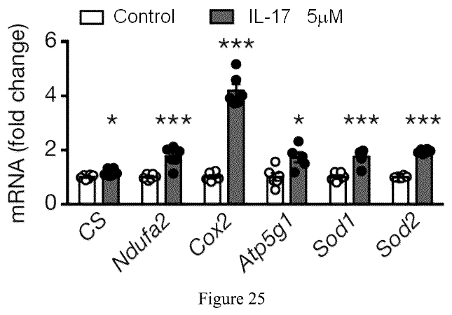

ACMSD inhibition with compound I-18 at 100 μM increases the mRNA levels of mitochondrial and oxidative stress genes; citrate synthase, CS; NDAH ubiquinone oxidoreductase subunit 2, Ndufa2, cytochrome c oxidase subunit 2, Cox2, ATP synthase lipid-binding protein, Atp5g1, superoxide dismutase 1, Sod1, and superoxide dismutase 2, Sod2 (FIG. 25).

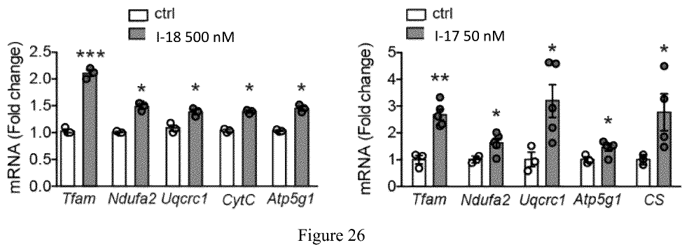

ACMSD inhibition with compounds I-17 and I-18, increases the mRNA levels of mitochondrial genes, TFAM, Ndufa, ubiquinol-cytochrome C reductase core protein 1, Uqcrc1, CytC, Atp5g1, and CS in mouse primary hepatocytes after 24 hr treatment (FIG. 26).

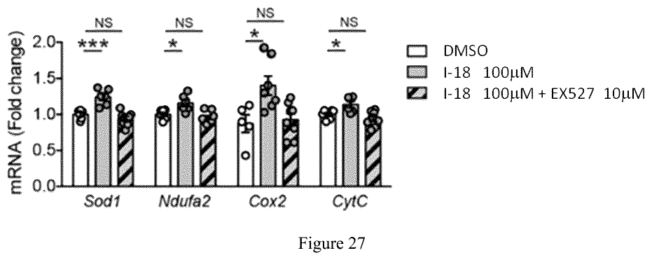

The increase in mRNA levels of mitochondrial genes, Sod1, Ndufa2, Cox2, and CytC, in HK-2 cells after 24 hr treatment with the ACMSD inhibitor, compound I-18, is SIRT1 dependent and is blocked by the inhibition of Sirt1 as shown by treatment with Compound I-18 in combination with the SIRT1 inhibitor, EX527, at the indicated concentrations (FIG. 27).

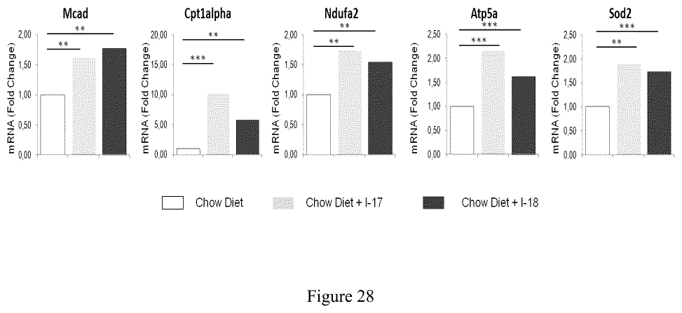

Administration of ACMSD inhibitors, compounds I-17 and I-18, also induces the transcription of the mitochondrial genes; medium-chain acyl-CoA dehydrogenase, Mcad, carnitine palmitoyltransferase 1, Cpt1a, NDAH ubiquinone oxidoreductase subunit 2, Ndufa2, ATP synthase lipid-binding protein, Atp5g1, and superoxide dismutase 2, Sod2 in the liver, whereas the expression of the same genes in the kidneys was unaffected (FIG. 28).

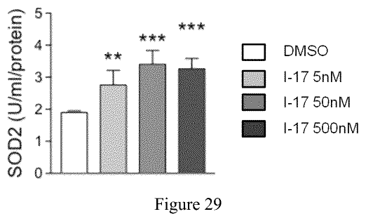

Inhibition of ACMSD with compound I-17 in primary hepatocytes increases the expression of mitochondrial SOD2 (FIG. 29).

### ACMSD Inhibition and Kupffer Cell (Marcophage) Polarisation

Inhibition of ACMSD with IL-18 induces polarisation of resident liver macrophages (Kupffer cells) towards an M2 macrophage phenotype, as shown by reduction of M1 macrophage gene markers and promotion of M2 macrophage gene markers.

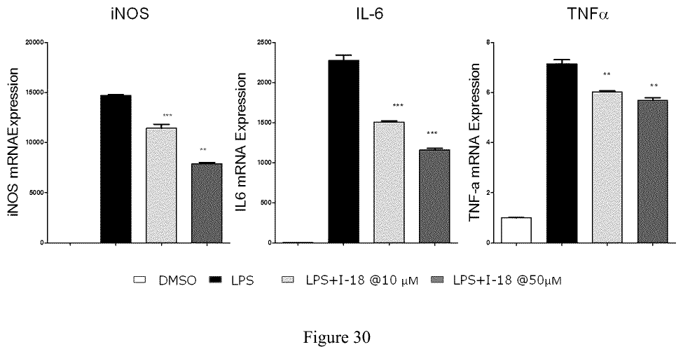

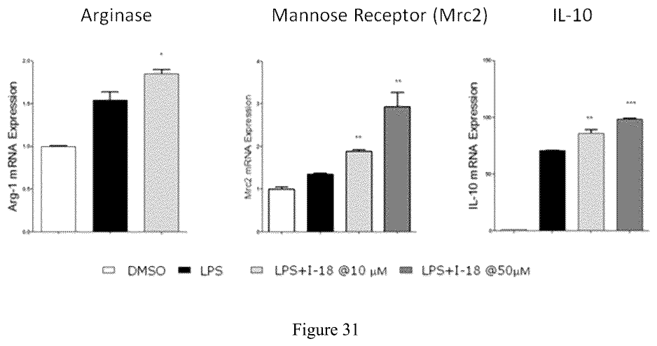

Thus murine Kupffer cells treated with LPS and Compound 1-18 show a decrease in the M1 phenotype gene markers, iNOS, IL-6, and TNFα (FIG. 30) and a corresponding increase in the M2 phenotype gene markers, Arginase-1, Mannose Receptor (Mrc-2), and IL-10 (FIG. 31).

ACMSD Inhibition and Protection from LPS-Induced Systemic Inflammation

Inhibition of ACMSD with IL-18 increases survival i.e. decreases mortality of C57BL/6 mice from LPS-induced systemic inflammation by promoting an anti-inflammatory phenotype.

Anti-inflammatory properties of ACMSD Inhibitors

α-Amino-β-Carboxymuconic-ε-Semialdehyde Decarboxylase (ACMSD) is an enzyme of Kynurenine Pathway (KP), mainly expressed in liver and kidney, which represents a branch point in the de novo NAD⁺ biosynthesis pathway that directs the conversion of tryptophan to NAD⁺.

We demonstrated that ACMSD inhibition by the pharmacological inhibitor Compound 1-18 showed protective effects in preclinical model of liver and kidney diseases, such as NASH and AKI respectively.

We established that inhibition of ACMSD, with Compound I-18, exhibits anti-inflammatory effects in vitro in liver macrophages promoting their conversion from pro-inflammatory M1 (induced by LPS) to anti-inflammatory M2 phenotype (M1-to-M2 transition), a mechanism of action in agreement with the metabolic reprogramming modulated by improved NAD⁺ and mitochondrial homeostasis.

Furthermore, these effects translate in an increased survival in LPS-induced and Cecal Ligation and Puncture (CLP)-induced systemic inflammation mouse models, expanding the therapeutic opportunities for ACMSD inhibitors in systemic inflammation-associated diseases of the liver-kidney axis.

MATERIALS AND METHODS: Treatment with Compound I-18 in presence of LPS for 24 h has been performed to evaluate anti inflammatory properties and M1 to M2 transition. Renal tubular cells were stimulated with Compound I-18 and TGFβ for 16 hrs to evaluate the expression of genes involved in fibrosis. Compound 1-18 (50 mg/kg) was dosed daily in mice treated with LPS 15 mg/kg. Survival percentage and liver gene modulation has been monitored in Sham groups, LPS and LPS+Compound 1-18 groups (n=8). 12-15 week old mice underwent CLP (50% ligation, thru-and-thru puncture with 21G needle) to evaluate CLP-induced sepsis. Compound I-18 was dosed at the concentration of 50 mg/kg for 7 days from CLP. Survival percentage and expression of genes involved in renal inflammation has been monitored.

RESULTS: ACMSD expression has been determined in liver resident macrophages (Kupffer cells). Pharmacological inhibition of ACMSD demonstrates anti-inflammatory properties in vitro promoting the anti-inflammatory M2 phenotype. Compound 1-18 reduced in vitro fibrosis in renal cells. Compound 1-18 promotes survival in mice treated with two different inflammatory stimuli (LPS and CLP).

CONCLUSIONS: Overall these results expand the therapeutic opportunities for ACMSD Inhibitors, revealing novel physiological functions of ACMSD in the modulation of the systemic inflammatory response and providing further therapeutic applications for ACMSD inhibitors.

Effect of ACMSD Inhibition on the Polarisation of M1 vs. M2 Macrophages

Macrophages are essential components of the innate immunity and play a central role in inflammation and host defense. Macrophages undergo classical M1 activation leading to the release of pro-inflammatory cytokines, ROS, and nitric oxide. Alternatively M2 activation can promote the tissue remodelling and exerts immune regulatory functions producing ornithine and polyamine. Macrophages can switch from an activated M1 state back to M2 due to their plasticity, and vice versa. Kupffer cell M1/M2 polarisation critically contributes to the pathogenesis of hepatic metabolic syndromes.

M1 phenotype shows pro-inflammatory properties. M2 phenotype exerts anti-inflammatory and immunotolerant effects.

M1 to M2 shift can be seen with inhibition of ACMSD by Compound 1-18 blocking of pro-inflammatory M1 macrophages. Murine Kupffer cells were treated with LPS and Compound I-18 to evaluate the role of ACMSD inhibition in M1/M2 transition. FIG. 30 shows that Compound I-18 blocks pro-inflammatory M1 Kupffer macrophages.

M1 to M2 shift can be seen with inhibition of ACMSD by Compound 1-18 by increase of anti-inflammatory M2 macrophages. M2 phenotype biomarkers should be increased during inflammation, indicating an anti-inflammatory response. FIG. 31 shows that Compound I-18 promotes anti-inflammatory M2 Kupffer macrophages.

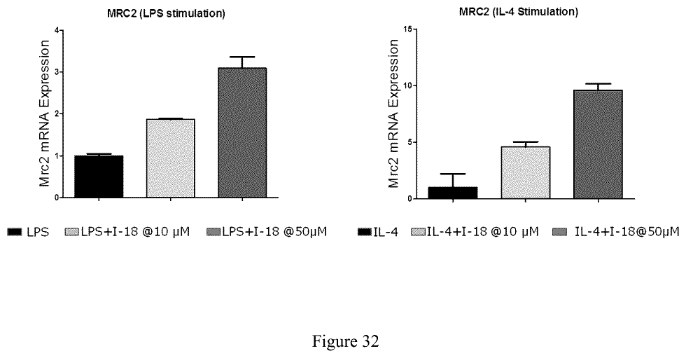

Also, two different stimuli (LPS and IL-4) were performed to evaluate M2 phenotype. FIG. 32 shows that Compound I-18 can promote M2 macrophage phenotype.

Accordingly, it is shown that Compound I-18 blocks pro-inflammatory M1 macrophages in dose-response manner in murine Kupffer cells. Compound I-18 promotes M2 macrophage phenotype in murine Kupffer cells. These results suggest that Compound I-18 may have anti-inflammatory effect, favoring anti-inflammatory M2 while reducing the pro-inflammatory M1 phenotype.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice of testing the present disclosure, suitable methods and materials are described below. All publications, patent applications, patents, and other references mentioned herein are hereby expressly incorporated by reference. The references cited herein are not admitted to be prior art of the claimed disclosure. In the case of conflict, the present specification, including definitions, will control. In addition, the materials, methods, and examples are illustrative only and are not intended to be limiting.

### EQUIVALENTS

Those skilled in the art will recognize, or be able to ascertain using no more than routine experimentation, many equivalents to the specific embodiments and methods described herein. Such equivalents are intended to be encompassed by the scope of the present disclosure.

1. A method of treating an acute inflammatory condition in a subject comprising administering to the subject a therapeutically effective amount of a compound of Formula (I): 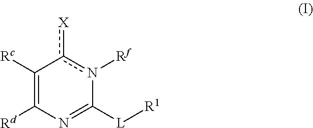
or a pharmaceutically acceptable salt or tautomer thereof,
wherein:
X is O or OH;
L is —(CH₂)ₘCH₂CH₂—, —(CH₂)ₘY(CH₂)ₚ—, —(CH₂)ₘC(O)(CH₂)ₚ—, —(CH₂)ₘC(O)O(CH₂)ₚ—, —(CH₂)ₘC(O)NR²(CH₂)ₚ—, or —(CH₂)ₘNR²C(O)(CH₂)ₚ;
Y is O, N or S(O)_{q};
R¹ is C₆-C₁₀ aryl or heteroaryl, wherein the aryl and heteroaryl are substituted with R^{a} and R^{b}, and optionally substituted with one or more R^{e};
R² is H or C₁-C₆ alkyl;
one of R^{a} and R^{b} is hydrogen and the other is —(CH₂)ᵣCO₂R^{x}, —OCH₂CO₂R^{x}, —(CH₂)ᵣtetrazole, —(CH₂)ᵣoxadiazolone, —(CH₂)ᵣtetrazolone, —(CH₂)ᵣdihydrotetrazolone, —(CH₂)ᵣthiadiazolol, —(CH₂)ᵣ isoxazol-3-ol, —(CH₂)ᵣP(O)(OH)OR^{x}, —(CH₂)ᵣS(O)₂OH, —(CH₂)ᵣC(O)NHCN, or —(CH₂)ᵣC(O)NHS(O)₂alkyl;
R^{c} is H, C₁-C₆ alkyl, C₁-C₆ haloalkyl, halogen, —CN, —OR^{x}, —CO₂R^{x}, or NO₂;
R^{d} is methyl, optionally substituted 5- to 10-membered aryl, optionally substituted 5- or 6-membered heteroaryl, or optionally substituted 5- or 6-membered carbocycle;
each R^{x} is independently at each occurrence hydrogen or C₁-C₆ alkyl;
each R^{e} is independently C₁-C₆ alkyl, C₂-C₆ alkenyl, C₂-C₆ alkynyl, halogen, —OR^{y}, C₁-C₆ haloalkyl, —NHR^{z}, —OH, or —CN;
R^{f} is H or absent;
each R^{y} and R^{z} is independently hydrogen, C₁-C₆ alkyl, or C₁-C₆ haloalkyl;
each m and p independently is 0, 1, or 2, wherein m+p<3;
q is 0, 1, or 2;
r is 0 or 1; and
the dotted line is an optional double bond;
with the proviso that R^{c} is not hydrogen or —CN when X is O, L is —SCH₂— and R^{d} is optionally substituted phenyl; that R^{c} is not C₁-C₆ alkyl when X is O, L is —SCH₂— and R^{d} is methyl; and that R^{x} is not —CN when X is O, L is —SCH₂— and R^{d} is 2-furyl.

2. The method of claim 1, wherein the compound is of Formula (Ia) 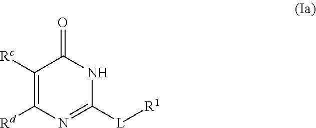
or a pharmaceutically acceptable salt, or tautomer thereof.

3. The method of claim 1, wherein the compound is of Formula (Ib): 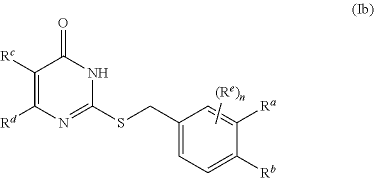
or a pharmaceutically acceptable salt thereof,
wherein:
one of R^{a} and R^{b} is hydrogen and the other is CO₂R^{x}, CH₂CO₂R^{x}, tetrazole, or oxadiazolone;
R^{c} is halogen, —CN, —OR^{x}, or C₁-C₆ alkyl;
R^{d} is methyl, optionally substituted 5- to 10-membered aryl, optionally substituted 5- or 6-membered heteroaryl, or optionally substituted 5- or 6-membered carbocycle; and
R^{x} is hydrogen or C₁-C₆ alkyl;
each R^{e} is independently C₁-C₆ alkyl, C₂-C₆ alkenyl, C₂-C₆ alkynyl, halogen, —OR^{y}, C₁-C₆ haloalkyl, —NHR^{z}, —OH, or —CN;
each R^{y} and R^{z} is independently hydrogen, C₁-C₆ alkyl, or C₁-C₆ haloalkyl; and
n is 0, 1, 2, or 3;
with the proviso that R^{c} is not hydrogen or —CN when R^{d} is optionally substituted phenyl and that R^{c} is not —CN when R^{d} is 2-furyl.

4. The method of claim 1,
wherein:
one of R^{a} and R^{b} is hydrogen and the other is CO₂R^{x}, CH₂CO₂R^{x}, tetrazole, or oxadiazolone;
R^{c} is halogen, —CN, —OR^{x}, or C₁-C₆ alkyl;
R^{d} is methyl, optionally substituted 5- to 10-membered aryl, optionally substituted 5- or 6-membered heteroaryl, or optionally substituted 5- or 6-membered carbocycle; and
R^{x} is hydrogen or C₁-C₆ alkyl;
each R^{e} is independently C₁-C₆ alkyl, C₂-C₆ alkenyl, C₂-C₆ alkynyl, halogen, —OR^{Y}, C₁-C₆ haloalkyl, —NHR^{z}, —OH, or —CN;
each R^{y} and R^{z} is independently hydrogen, C₁-C₆ alkyl, or C₁-C₆ haloalkyl; and
n is 0, 1, 2, or 3;
with the proviso that R^{c} is not hydrogen or —CN when R^{d} is optionally substituted phenyl and that R^{c} is not —CN when R^{d} is 2-furyl.

5. The method of claim 1, wherein the compound is represented by Formula (II): 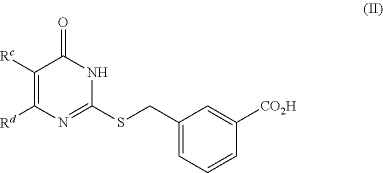
or a pharmaceutically acceptable salt thereof.

6. The method of claim 5, wherein R^{c} is halogen, —CN, —OR^{x}, or C₁-C₆ alkyl, R^{d} is methyl, optionally substituted 5- to 10-membered aryl, optionally substituted 5- or 6-membered heteroaryl, or optionally substituted 5- or 6-membered carbocycle, and R^{x} is hydrogen or C₁-C₆ alkyl.

7. The method of claim 1, wherein R^{c} is —CN or halogen.

8. The method of claim 1, wherein R^{d} is methyl, cyclohexyl, pyridinyl, thiazolyl, phenyl, or thienyl.

9. The method of claim 1, wherein R^{d} is methyl, cyclohexyl, pyridinyl, thiazolyl, thienyl, or optionally substituted phenyl.

10. The method of claim 1, wherein R^{a} is hydrogen, CH₂CO₂H, tetrazole, or oxadiazolone (1,2,4-oxadiazol-5(4H)-one).

11. The method of claim 1, wherein R^{b} is hydrogen, CH₂CO₂H, tetrazole, or oxadiazolone (1,2,4-oxadiazol-5(4H)-one).

12. The method of claim 1, wherein n is 0.

13. The method of claim 1, wherein the compound is selected from the group consisting of 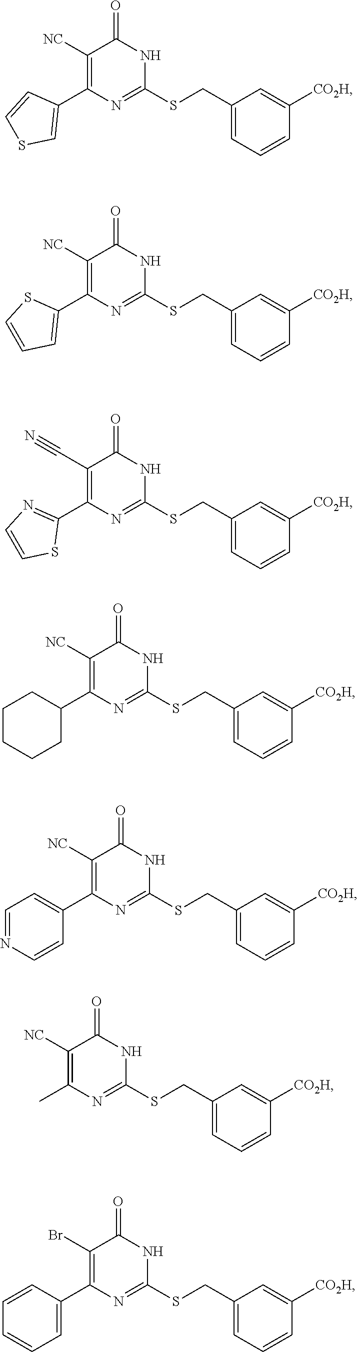 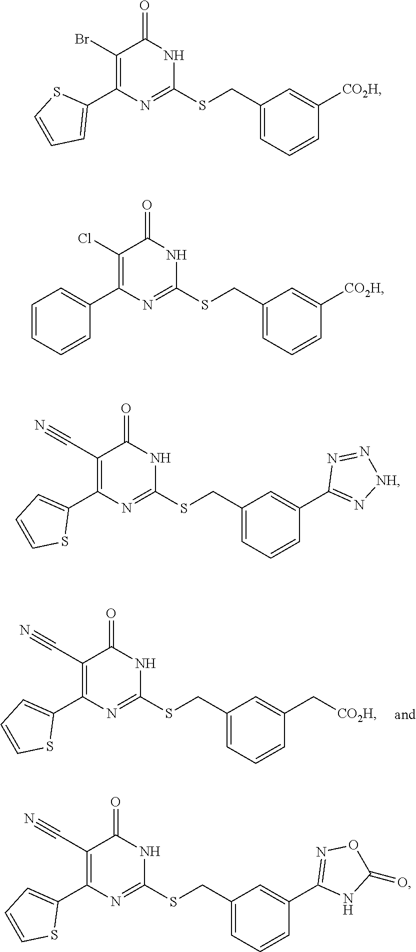 [SMILES: O=C(O)c1cccc(CSc2nc(-c3cccs3)c(Br)c(=O)[nH]2)c1 ; O=C(O)c1cccc(CSc2nc(-c3ccccc3)c(Cl)c(=O)[nH]2)c1 ; N#Cc1c(-c2cccs2)nc(SCc2cccc(-c3nn[nH]n3)c2)[nH]c1=O ; N#Cc1c(-c2cccs2)nc(SCc2cccc(CC(=O)O)c2)[nH]c1=O ; N#Cc1c(-c2cccs2)nc(SCc2cccc(-c3noc(=O)[nH]3)c2)[nH]c1=O]
or a pharmaceutically acceptable salt thereof.

14. The method of claim 1, wherein the compound is selected from the group consisting of 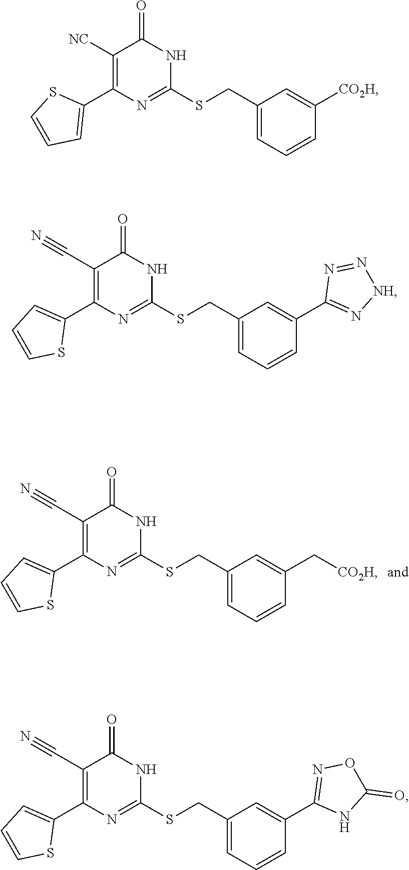 [SMILES: N#Cc1c(-c2cccs2)nc(SCc2cccc(C(=O)O)c2)[nH]c1=O ; N#Cc1c(-c2cccs2)nc(SCc2cccc(-c3nn[nH]n3)c2)[nH]c1=O ; N#Cc1c(-c2cccs2)nc(SCc2cccc(CC(=O)O)c2)[nH]c1=O ; N#Cc1c(-c2cccs2)nc(SCc2cccc(-c3noc(=O)[nH]3)c2)[nH]c1=O]
or a pharmaceutically acceptable salt thereof.

15. The method of claim 1, wherein the compound is selected from the group consisting of 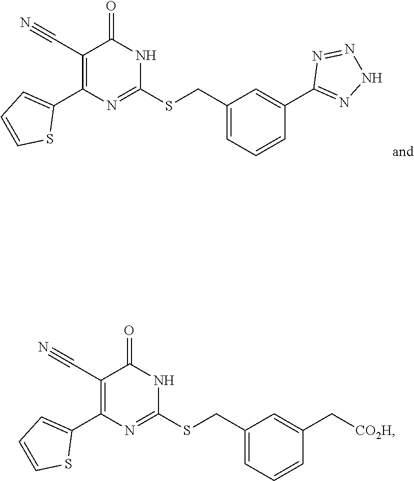
or a pharmaceutically acceptable salt thereof.

16. The method of claim 1, wherein the compound is 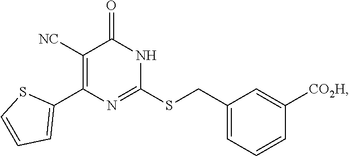 [SMILES: N#Cc1c(-c2cccs2)nc(SCc2cccc(C(=O)O)c2)[nH]c1=O]
or a pharmaceutically acceptable salt thereof.

17. The method of claim 1, wherein the compound is 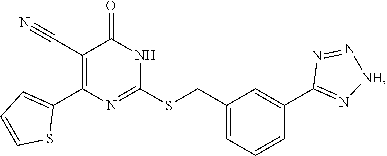
or a pharmaceutically acceptable salt thereof.

18. The method of claim 1, wherein the compound is 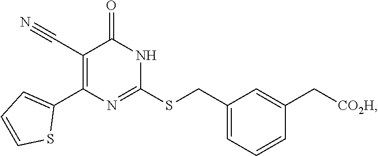 [SMILES: N#Cc1c(-c2cccs2)nc(SCc2cccc(CC(=O)O)c2)[nH]c1=O]
or a pharmaceutically acceptable salt thereof.

19. The method of claim 1, wherein the compound is 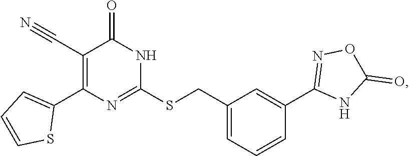 [SMILES: N#Cc1c(-c2cccs2)nc(SCc2cccc(-c3noc(=O)[nH]3)c2)[nH]c1=O]
or a pharmaceutically acceptable salt thereof.

20. The method of claim 1, wherein the acute inflammatory condition is a systemic inflammatory condition.

21. The method of claim 1, wherein the acute inflammatory condition is an organ-specific condition.

22. The method of claim 1, wherein the acute inflammatory condition is a cytokine storm or hypercytokinemia, systemic inflammatory response syndrome (SIRS), graft versus host disease (GVHD), acute respiratory distress syndrome (ARDS), severe acute respiratory distress syndrome (SARS), catastrophic anti-phospholipid syndrome, viral infections, bacterial infections, fungal infections, influenza, pneumonia, shock, or sepsis.

23. The method of claim 1, wherein the acute inflammatory condition is acute pancreatitis, hepatitis, respiratory condition, or enterocolitis.

24. The method of claim 1, wherein the method reduces a pro-inflammatory cytokine or increases an anti-inflammatory cytokine.

25. The method of claim 24, wherein the pro-inflammatory cytokine is IL-1β, IL-6, IL-18, TNF-α, or TGF-β.

26. The method of claim 24, wherein pro-inflammatory cytokine is MCP-1, TNF-α, or IL-1β.

27. The method of claim 24, wherein pro-inflammatory cytokine is IL-6.

28. The method of claim 24, wherein the anti-inflammatory cytokine is IL-10.

29. The method of claim 1, wherein expression of sirtuin-1 modulated genes sod2, tfam, dda1 genes are increased in the liver.

30-116. (canceled)
